(19) United States
(12) Patent Application Publication
(10) Pub. No.: US 2025/0177743 A1
(43) Pub. Date: Jun. 5, 2025

(54) TUMOR TREATING FIELDS SYSTEM AND ALTERNATING CURRENT SIGNAL APPLICATION METHOD THEREOF

(71) Applicant: JIANGSU HEALTHY LIFE INNOVATION MEDICAL TECHNOLOGY CO., LTD, Jiangsu (CN)

(72) Inventors: Sheng CHEN, Jiangsu (CN); Jing YU, Jiangsu (CN); Qichao SHEN, Jiangsu (CN); Jianjun YING, Jiangsu (CN); Jun ZHANG, Jiangsu (CN); Hu SUN, Jiangsu (CN); Yidong SUN, Jiangsu (CN); Jiajie HUI, Jiangsu (CN)

(21) Appl. No.: 18/723,196

(22) PCT Filed: Dec. 19, 2022
(86) PCT No.: PCT/CN2022/140141

§ 371 Date: Feb. 10, 2025

(30) Foreign Application Priority Data
Dec. 22, 2021 (CN) 202111578521.1
Dec. 22, 2021 (CN) 202111578561.6
Dec. 22, 2021 (CN) 202111578597.4
Dec. 22, 2021 (CN) 202111580036.8
Dec. 22, 2021 (CN) 202111580039.1
Dec. 22, 2021 (CN) 202111580121.4
Dec. 22, 2021 (CN) 202111580130.3
Dec. 22, 2021 (CN) 202111580142.6
Dec. 22, 2021 (CN) 202111580196.2
Dec. 22, 2021 (CN) 202111580208.1
Dec. 22, 2021 (CN) 202123242599.4
Dec. 22, 2021 (CN) 202123242623.4
Dec. 24, 2021 (CN) 202111596993.X
Dec. 24, 2021 (CN) 202111599376.5

(51) Int. Cl. A61N 1/36; A61N 1/02; A61N 1/04

(52) U.S. Cl. CPC A61N 1/36002; A61N 1/025; A61N 1/0408; A61N 1/36034

(57) ABSTRACT

A tumor treating fields system includes an electric field generator and at least two pairs of insulated electrodes, the electric field generator includes an alternating current signal generator, generating at least two alternating current signals, the at least two alternating current signals are output to the at least two pairs of insulated electrodes, to generate alternating electric fields in at least two directions between the at least two pairs of insulated electrodes; and a signal controller, obtaining temperature information of the insulated electrodes, and individually control output of each alternating current signal based on the temperature information, to selectively apply to corresponding insulated electrodes an alternating current signal that generates a corresponding alternating electric field of the alternating electric fields in the at least two directions. The electric field generator of the present tumor treating fields system individually controls the output of the alternating current signals through the signal controller.

### CROSS-REFERENCE TO RELATED APPLICATION(S)

The present application is a US national application of International application No. PCT/CN2022/140141 filed on Dec. 19, 2022 which claims priority to Chinese Patent Application No. 202111596993.X field on Dec. 24, 2021, Chinese Patent Application No. 202111599376.5 field on Dec. 24, 2021, Chinese Patent Application No. 202111580208.1 field on Dec. 22, 2021, Chinese Patent Application No. 202111578597.4 field on Dec. 22, 2021, Chinese Patent Application No. 202111580130.3 field on Dec. 22, 2021, Chinese Patent Application No. 202111580121.4 field on Dec. 22, 2021, Chinese Patent Application No. 202111580142.6 field on Dec. 22, 2021, Chinese Patent Application No. 202111580036.8 field on Dec. 22, 2021, Chinese Patent Application No. 202111578521.1 field on Dec. 22, 2021, Chinese Patent Application No. 202111580039.1 field on Dec. 22, 2021, Chinese Patent Application No. 202123242599.4 field on Dec. 22, 2021, Chinese Patent Application No. 202111580196.2 field on Dec. 22, 2021, Chinese Patent Application No. 202111578561.6 field on Dec. 22, 2021, and Chinese Patent Application No. 202123242623.4 field on Dec. 22, 2021, the entire disclosures of which are incorporated herein by reference.

### FIELD

The present disclosure relates to Tumor Treating Fields (TTF) technology, in particular to an electric field generator and an apparatus for applying an electric field to a subject, and an alternating current signal applied method thereof.

### BACKGROUND

Tumor Treating Fields (TTF) therapy is a treatment that uses low-intensity intermediate frequency (for example, 100˜300 kHz) alternating electric fields to prevent the formation of spindle microtubules during mitosis of certain tumor cells and inhibit the separation of intracellular organelles during cell division, inducing cell apoptosis during mitosis, and thereby achieving the effect of treating tumors.

Compared with traditional cancer treatments, TTF has an innovative mechanism of action. Some physiological characteristics of tumor cells, such as geometric shape and high frequency of mitosis, make the tumor cells vulnerable to TTF. TTF disrupts the normal aggregation of tubulin by exerting directional forces on intracellular polar particles, such as macromolecules and organelles. These processes may lead to physical disruption of cell membranes and cell apoptosis. At the end of cell mitosis, the structure of the cleavage furrow will cause uneven distribution of the electric field around the cells. At the same time, under the influence of TTF, the electric field intensity at the cleavage furrow is significantly enhanced. The charged substances in the cells move toward the cleavage furrow; causing interference or even destruction of the formation of the cell structure, which may ultimately lead to cell division failure and apoptosis.

In the related art, TTF applies an alternating electric field to a location on a subject's skin adjacent to a tumor via an electric field generator. Due to the blessing of the alternating electric field, the heat on the surface of the subject rises. In order to avoid cryogenic burns to the skin, it is necessary to design an electric field generator, an apparatus and a method for applying alterlating current signal based on the detection of the temperature of the subject's surface.

### SUMMARY

The present disclosure provides a tumor treating fields system, an electric field generator and a method for dynamically adjusting an applied alternating electrical signal.

According to an aspect of the present disclosure, a tumor treating fields system is provided. The tumor treating fields system includes an electric field generator and at least two pairs of insulated electrodes, wherein the electric field generator comprises: an alternating current signal generator, configured to generate at least two alternating current signals, wherein the at least two alternating current signals are output to the at least two pairs of insulated electrodes, so as to generate alternating electric fields in at least two directions between the at least two pairs of insulated electrodes; and a signal controller, configured to obtain temperature information of the insulated electrodes, and individually control output of each of the at least two alternating current signals based on the temperature information, so as to selectively apply the corresponding alternating current signal to corresponding insulated electrodes to generate a corresponding alternating electric field.

Optionally, the alternating current signal generator comprises: a direct current signal source, configured to generate a direct current signal; and a signal converter, configured to convert the direct current signal into the at least two alternating current signals.

Optionally, the alternating current signal generator further comprises: a direct current signal switch, electrically connected between the direct current signal source and the signal converter, wherein, the signal controller is configured to control a supply of the direct current signal from the direct current signal source to the signal converter by controlling the direct current signal switch.

Optionally, the tumor treating fields system further comprises: at least two pairs of output terminals, wherein each pair of output terminals is configured for supplying corresponding alternating current signal of the at least two alternating current signals from the alternating current signal generator.

Optionally, the tumor treating fields system further comprises: at least two pairs of switches, electrically connected to the at least two pairs of output terminals respectively, wherein, the signal controller is configured to individually control output of the at least two alternating current signals from the at least two pairs of output terminals by individually controlling the at least two pairs of switches.

Optionally, the signal controller is configured to: monitor the obtained temperature information according to each of the insulated electrodes; in response to the temperature information being greater than a temperature threshold, control to stop outputting the alternating current signal applied to the corresponding insulated electrode among the at least two alternating current signals; and in response to the temperature information being less than or equal to the temperature threshold, control to output the alternating current signal applied to the corresponding insulated electrode among the at least two alternating current signals.

Optionally, a range of the temperature threshold is 37° C.-41° C.

Optionally, the tumor treating fields system further comprises at least two pairs of temperature sensor array's, and each temperatur sensor arrays is configured to sense temperature signals of the insulated electrodes, so as to provide corresponding temperature information.

Optionally, the tumor treating fields system further comprises an adapter configured to convert the temperature signals into the temperature information and transmit the at least two alternating current signals to the at least two pairs of the insulated electrodes.

According to an aspect of the present disclosure, a method of applying alternating electrical signals with the above-mentioned tumor treating fields system is provided. The method comprises: step 1: obtaining temperature information of the insulated electrodes; and step 2: based on the temperature information, individually controlling output of each of the at least two alternating current signals, so as to selectively apply the corresponding alternating current signal to the corresponding insulated electrodes to genterate a corresponding alternating electric field.

Optionally, the step 2 specifically comprises the following steps: step 21: comparing a first temperature information with a temperature threshold, wherein the first temperature information is temperature information corresponding to an obtained temperature signal monitored by the insulated electrode that generates a first electric field of the alternating electric fields in the at least two directions; and step 22: in response to the first temperature information being greater than the temperature threshold, controlling to stop output of a first alternating current signal of the at least two alternating current signals to the insulated electrodes that generate the first electric field; or step 23: in response to the first temperature information not being greater than the temperature threshold, controlling to continuously output the first alternating current signal to the insulated electrodes that generate the first electric field.

Optionally, a range of the temperature threshold is 37° C.-41° C.

Optionally, the step 2 further comprises the following steps: step 24: comparing a second temperature information with the temperature threshold, wherein the second temperature information is temperature information corresponding to an obtained temperature signal monitored by the insulated electrodes that generate a second electric field of the alternating electric fields in the at least two directions; and step 25: in response to the second temperature information being greater than the temperature threshold, controlling to stop the output of a second alternating current signal of the at least two alternating current signals to the insulated electrodes that generate the second electric field; or step 26: in response to the second temperature information not being greater than the temperature threshold, controlling to continuously output the second alternating current signal to the insulated electrodes that generate the second electric field.

Optionally, a range of the temperature threshold is 37° C.-41° C.

According to another aspect of the present disclosure, a computer-readable storage medium having instructions stored thereon is provided. The instructions, when being executed by the signal controller of the electric field generator of the above-mentioned tumor treating fields system, cause the electric field generator performs the above-mentioned method.

According to another aspect of the present disclosure, a computer program product is provided. The computer program product comprises instructions. The instructions, when being executed by the signal controller of the electric field generator of the above-mentioned tumor treating fields system, cause the electric field generator performs the above-mentioned method.

According to another aspect of the present disclosure, the tumor treating fields system further comprises: a plurality of insulated electrodes; a plurality of temperature sensor arrays, configured to sense temperature signals of the insulated electrodes to provide corresponding temperature information; a first cable, configured to provide a plurality of alternating electric field signal paths and a plurality of temperature signal paths; and an adapter, configured to: transmit a plurality of alternating electric field signals generated by the electric field generator to the corresponding insulated electrodes of the plurality of insulated electrodes via the plurality of alternating electric field signal paths; and receive the corresponding temperature signals transmitted in parallel via the plurality of temperature signal paths, and transmit a plurality of temperature values corresponding to the temperature signals to the electric field generator, so that the electric field generator controls the plurality of alternating electric field signals based on the plurality of temperature values, wherein the temperature signals are analog signals, and the temperature values are digital signals.

Optionally, the adapter comprises: an analog-to-digital converter, configured to convert the corresponding temperature signals into digital signals; and a signal processor, configured to calculate and store the plurality of temperature values based on the digital signals.

Optionally, each temperature sensor array of the plurality of temperature sensor arrays comprises a plurality of thermistors.

Optionally, the adapter further comprises a voltage regulator and a plurality of precision resistors, wherein the plurality of precision resistors are electrically connected between the voltage regulator and corresponding thermistors of the plurality of thermistors.

Optionally, the adapter further comprises a buffer, wherein the buffer comprises: a plurality of input terminals, electrically connected to corresponding thermistors of the plurality of thermistors; and a plurality of output terminals, electrically connected to corresponding input terminals of the plurality of input terminals of the analog-to-digital converter.

Optionally, the plurality of output terminals of the buffer are further electrically connected to corresponding precision resistors of the plurality of precision resistors one by one.

Optionally, the converter comprises a serial communication circuit, wherein the serial communication circuit is configured to serially transmit the plurality of temperature values to the electric field generator.

Optionally, a second cable is further comprised, wherein the second cable is configured to transmit a plurality of alternating electric field signals from the electric field generator to the adapter.

Optionally, a first connector is further comprised, wherein the first connector is mechanically and electrically connected between the first cable and the adapter.

Optionally, the first connector comprises a press-type spring connector.

Optionally, a second connector is further comprised, wherein the second connector is configured to mechanically and electrically connect the second cable to the electric field generator.

Optionally, the second connector comprises a press-type spring connector.

Optionally, the insulated electrode comprises: a plurality of electrode units, arranged in an array; a plurality of connecting parts, configured to connect two adjacent electrode units; and a wiring part, extended by a connecting part. The electrode units are provided with dielectric elements. Two opposite ends of the connecting parts are provided with conductive pads electrically connected to the corresponding dielectric elements. The plurality of connecting parts are located between two adjacent electrode units arranged in a column and are located between two adjacent electrode units arranged in a row, wherein a length of the connecting part between the two adjacent electrode units arranged in the column is less than that of the connecting part between the two adjacent electrode units arranged in the row. There are at least two connecting parts of the plurality of connecting parts are located between two adjacent electrode units arranged in the row. The conductive pad has a plurality of conductive cores arranged symmetrically at intervals and welded with the dielectric elements.

Optionally, the plurality of conductive cores of the conductive pad are center-symmetrically arranged on the connecting part, wherein a center of the conductive pad is located on a center line of the dielectric element.

Optionally, the plurality of conductive cores of the conductive pad are axial-symmetrically arranged on the connecting part, and expose a side of the connecting part facing the dielectric element.

Optionally, each conductive core comprises an inner arc and an outer arc which are connected end to end, wherein the inner arc and the outer arc of the conductive core are axial-symmetrically arranged.

Optionally, outer arcs of the plurality of conductive cores of the conductive pad are located on a same circumference.

Optionally, the insulated electrode further comprises a backing supporting the electrode unit.

Optionally, the backing has a plurality of concave corners for preventing wrinkling, wherein the concave corners are located at corners of the backing and connect to the exterior.

Optionally, the concave corners are formed by edges at the corners of the backing being recessed inwards, wherein an angle between two sides forming the concave corner of the backing is not less than 90 degrees.

Optionally, the insulated electrode further comprises a support member surrounding the electrode unit, wherein the support member has a through hole disposed therethrough and configured to accommodate the electrode unit.

Optionally, the insulated electrode further comprises a hygroscopic element arranged between the electrode units.

Optionally, the support member has an opening extending therethrough for accommodating the hygroscopic element, wherein the opening and the through hole are arranged at intervals.

Optionally, the electrode unit further comprises a temperature sensor, wherein the dielectric element has a perforation extending therethrough for accommodating the temperature sensor.

Optionally, the connecting part has an insulating substrate and a plurality of conductive traces embedded in the insulating substrate, wherein the conductive pads located at the opposite ends of the connecting part are electrically connected with one conductive trace.

Optionally, the electrode units are arranged in three rows and three columns, wherein a number of the electrode units is nine.

Optionally, the insulated electrode comprises: a flexible circuit board; and a plurality of dielectric elements, arranged on the flexible circuit board, wherein the plurality of dielectric elements are arranged in at least three rows and four columns. Spacings between two adjacent dielectric elements arranged in rows are different or spacings between two adjacent dielectric elements arranged in columns are different.

Optionally, spacings between two adjacent dielectric elements in adjacent columns within the same row are the same, and spacings between two adjacent dielectric elements in spaced columns within the same row are the same.

Optionally, the spacing between the adjacent dielectric elements in adjacent columns within the same row is smaller than the spacing between two adjacent dielectric elements in spaced columns within the same row.

Optionally, the spacing between the adjacent dielectric elements in adjacent rows within the same column is smaller than the spacing between two adjacent dielectric elements in spaced rows within the same column.

Optionally, the spacing between the adjacent dielectric elements in adjacent columns within the same row is equal to the spacing between the adjacent dielectric elements in adjacent rows within the same column.

Optionally, the dielectric elements are arranged in three rows and five columns, wherein a number of the dielectric elements is 14.

Optionally, the insulated electrode further comprises: an insulating plate, arranged on the flexible circuit board, wherein the insulating plate and the dielectric element are respectively arranged on two opposite sides of the flexible circuit board.

Optionally, the insulated electrode further comprises: a plurality of temperature sensors, arranged on the flexible circuit board, wherein the temperature sensors and the dielectric elements are located at the same side of the flexible circuit board.

Optionally, the insulated electrode further comprises: a backing, arranged on the flexible circuit board, wherein the backing and the dielectric elements are respectively arranged on two opposite sides of the flexible circuit board.

Optionally, the insulated electrode comprises: a flexible circuit board; a dielectric element and a temperature sensor arranged on a same side of the flexible circuit board; and a wire, electrically connected with the flexible circuit board. The temperature sensor has a ground terminal and a signal terminal. The flexible circuit board has an insulating substrate and three conductive traces embedded in the insulating substrate; wherein one of the three conductive traces is electrically connected with the dielectric element, one conductive trace is electrically connected with the ground terminal of the temperature sensor, and one conductive trace is electrically connected with the signal terminal of the temperature sensor. The wire is electrically connected with the three conductive traces of the flexible circuit board.

Optionally, the flexible circuit board has three golden fingers exposed from the insulating substrate and electrically connected with the corresponding parts of the wire.

Optionally, the three golden fingers are respectively electrically connected with a conductive trace of the flexible circuit board.

Optionally, the flexible circuit board is configured with conductive pads corresponding to the dielectric elements, wherein the conductive pads are welded with the dielectric elements.

Optionally, the conductive pad exposes the insulating substrate and connected to a conductive trace of the flexible circuit board that electrically connected with the dielectric element.

Optionally, the conductive pad comprises a plurality of conductive cores arranged at intervals, wherein the plurality of conductive cores are connected in series by a conductive trace of the flexible circuit board that electrically connected with the dielectric element.

Optionally, the flexible circuit board is configured with two pads, exposing the insulating substrate and corresponding to the temperature sensor.

Optionally, one of the two pads is welded with the ground terminal of the temperature sensor, and the other pad of the two pads is welded with the signal terminal of the temperature sensor.

Optionally, one of the two pads is connected to a conductive trace of the flexible circuit board that electrically connected with the ground terminal of the temperature sensor, and the other pad is connected to a conductive trace of the flexible circuit board that electrically connected with the signal terminal of the temperature sensor.

Optionally, one end of the wire is electrically connected with the flexible circuit board, and the other end is provided with a plug.

Optionally, a heat shrinkable sleeve is arranged at a joint of the wire and the flexible circuit board.

Optionally, the dielectric element is configured with a perforation extending therethrough, wherein the temperature sensor is accommodated in the perforation.

Optionally, a conductive trace of the three conductive traces that is electrically connected with the dielectric element is a first conductive trace, a conductive trace that is electrically connected with the ground terminal of the temperature sensor is a second conductive trace, and a conductive trace that is electrically connected with the signal terminal of the temperature sensor is a third conductive trace; and wherein the flexible circuit board is configured with a conductive pad that connected with the first conductive trace, and the flexible circuit board is further configured with two pads, wherein one of the two pads is connected with the second conductive trace and the other pad is connected with the third conductive trace.

Optionally, the conductive pad and the pads are arranged on the same side of the flexible circuit board.

Optionally, both the conductive pad and the two pads expose the insulating substrate of the flexible circuit board.

Optionally, the flexible circuit board further has three golden fingers welded with the wire, wherein the golden fingers expose the insulating substrate of the flexible circuit board.

Optionally, the golden fingers, the conductive pad and the two pads are located at the same side of the flexible circuit board.

Optionally, the insulated electrode further comprises a backing adhered to a corresponding part of the flexible circuit board.

Optionally, the insulated electrode further comprises an insulating plate arranged at a side of the flexible circuit board far away from the dielectric element, wherein the insulating plate corresponds to the dielectric element along the thickness direction and is sandwiched between the flexible circuit board and the backing.

Optionally, the insulated electrode comprises: a flexible circuit board, a single dielectric element electrically connected with the flexible circuit board and a plurality of temperature sensors. The number of the temperature sensors is n, wherein n is an integer greater than 1 and not greater than 8, and wherein the temperature sensor has a ground terminal and a signal terminal and the flexible circuit board has an insulating substrate and a plurality of conductive traces embedded in the insulating substrate, wherein the plurality of conductive traces are n+2 conductive traces. One of the conductive traces is electrically connected with the dielectric element, one conductive trace is electrically connected with ground terminals of all the temperature sensors, and the rest conductive traces are electrically connected with corresponding signal terminals of the temperature sensors respectively.

Optionally, the flexible circuit board has a wiring part electrically connected with both the dielectric element and the temperature sensor, wherein both the dielectric element and the temperature sensor are located at one end of the wiring part.

Optionally, the insulated electrode further comprises: a wire, wherein one end of the wire is electrically connected with the wiring part of the flexible circuit board, and wherein the wire and the dielectric element are located at opposite ends of the wiring part respectively.

Optionally, one end of the wire is electrically connected with the wiring part of the flexible circuit board, and the other end of the wire is provided with a plug.

Optionally, the flexible circuit board is configured with a conductive pad welded with the dielectric element, wherein the conductive pad is arranged at one end of the wiring part.

Optionally, the conductive pad exposes the insulating substrate and connected to a conductive trace of the flexible circuit board that electrically connected with the dielectric element.

Optionally, the n temperature sensors are all arranged in an area surrounded by conductive pads, wherein an extension direction of a straight line where the n temperature sensors are located is consistent with an extension direction of the wiring part.

Optionally, the conductive pad comprises a plurality of conductive cores arranged at intervals, wherein the plurality of conductive cores are connected in series by a conductive trace of the flexible circuit board that is electrically connected with the dielectric element.

Optionally, the plurality of conductive cores are arranged in a matrix at intervals, wherein, among the plurality of conductive cores, four conductive cores located in adjacent rows and adjacent columns are center-symmetrically arranged.

Optionally, the n temperature sensors are respectively arranged offset from the symmetrical center of the four conductive cores corresponding to the conductive pad.

Optionally, there are two temperature sensors, wherein one of the two temperature sensors is arranged on a side of the symmetrical center of the corresponding four conductive cores away from the wiring part, and the other is arranged on a side of the symmetrical center of the corresponding four conductive cores close to the wiring part.

Optionally, the flexible circuit board is configured with n pairs of pads corresponding to the temperature sensors and located at one end of the wiring part, wherein the n pairs of pads and the conductive pad are located at the same end of the wiring part.

Optionally, each pair of pads comprises a first pad and a second pad, wherein the first pad is welded with a ground terminal of the corresponding temperature sensor, and the second pad is welded with a signal terminal of the corresponding temperature sensor.

Optionally, each pair of pads is arranged offset from the symmetrical center of the corresponding four conductive cores.

Optionally, there are two pairs of pads, wherein one pair of pads is arranged on a side of the symmetrical center of the corresponding four conductive cores away from the wiring part, and the other pair of pads is arranged on a side of the symmetrical center of the corresponding four conductive cores close to the wiring part.

Optionally, a straight line where the symmetry center of each pair of then pairs of pads is located is parallel to an extension direction of the wiring part.

Optionally, the first pad is connected to a conductive trace of the flexible circuit board that is electrically connected with the ground terminal of the temperature sensor, and the second pad is respectively connected to a corresponding conductive trace of the flexible circuit board that is electrically connected with the signal terminal of the corresponding temperature sensor.

Optionally, the dielectric element has a perforation arranged corresponding to the temperature sensor, wherein the temperature sensor is accommodated in the corresponding perforation.

Optionally, the number of the temperature sensors is two, the number of the conductive traces is four, and the number of the conductive cores is six.

Optionally, the insulated electrode further comprises: a backing, adhered to a corresponding part of the flexible circuit board.

Optionally, the insulated electrode further comprises: an insulating plate, arranged opposite to the dielectric element, wherein the insulating plate is arranged corresponding to the dielectric element along the thickness direction. The insulating plate is sandwiched between the dielectric element and the backing.

Optionally, the insulated electrode comprises: a flexible circuit board; a dielectric element and a plurality of temperature sensors both arranged at the same side of the flexible circuit board; and a wire, electrically connected with the flexible circuit board. The number of the temperature sensors is n, wherein n is an integer greater than 1 and not greater than 8. Each temperature sensor has a ground terminal and a signal terminal. The flexible circuit board has an insulating substrate and a plurality of conductive traces embedded in the insulating substrate, wherein the plurality of conductive traces are n+2 conductive traces. One of the conductive traces is electrically connected with the dielectric element, one conductive trace is electrically connected with ground terminals of all the temperature sensors, and the rest conductive traces are electrically connected with corresponding signal terminals of the temperature sensors respectively. The wire is electrically connected with the plurality of conductive traces of the flexible circuit board.

Optionally, the flexible circuit board has a plurality of golden fingers exposing the insulating substrate and electrically connected with the corresponding parts of the wire.

Optionally, the golden fingers are respectively electrically connected with a conductive trace of the flexible circuit board.

Optionally, the number of the temperature sensors is two, the number of the conductive traces is four, and the number of the golden fingers is four.

Optionally, the flexible circuit board is configured with conductive pads corresponding to the dielectric elements, wherein the conductive pads are welded with the dielectric elements.

Optionally, the conductive pad exposes the insulating substrate and is connected to a conductive trace of the flexible circuit board that is electrically connected with the dielectric element.

Optionally, the conductive pad comprises a plurality of conductive cores arranged at intervals, wherein the plurality of conductive cores are connected in series by a conductive trace of the flexible circuit board that is electrically connected with the dielectric element.

Optionally, the flexible circuit board is configured with n pairs of pads, wherein each pair of pad is located between two corresponding conductive cores arranged at intervals.

Optionally, each pair of pads is arranged on a position of the flexible circuit board corresponding to the corresponding temperature sensor, wherein each pair of pads exposes the insulating substrate of the flexible circuit board.

Optionally, each pair of pads comprises a first pad and a second pad, wherein the first pad is welded with a ground terminal of the corresponding temperature sensor, and the second pad is welded with a signal terminal of the corresponding temperature sensor.

Optionally, the first pad is connected to a conductive trace of the flexible circuit board that is electrically connected with the ground terminal of the temperature sensor, and the second pad is respectively connected to a corresponding conductive trace of the flexible circuit board that is electrically connected with the signal terminal of the corresponding temperature sensor.

Optionally, one end of the wire is electrically connected with the flexible circuit board, and the other end of the wire is provided with a plug.

Optionally, a heat shrinkable sleeve is arranged at a joint of the wire and the flexible circuit board.

Optionally, the dielectric element has a perforation arranged corresponding to the temperature sensor, wherein the temperature sensor is accommodated in the corresponding perforation.

Optionally, a conductive trace of the plurality of conductive traces that is electrically connected with the dielectric element is a first conductive trace, a conductive trace that is electrically connected with the ground terminal of the temperature sensor is a second conductive trace, and the rest n conductive traces that are respectively electrically connected with signal terminals of the corresponding temperature sensor are third conductive traces. The flexible circuit board is configured with a conductive pad that is connected with the first conductive trace. The flexible circuit board is configured with n pairs of pads, wherein one pad of each pair of pads is connected with the second conductive trace and the other pad is connected with the corresponding third conductive trace.

Optionally, the conductive pad and the pads are arranged on the same side of the flexible circuit board.

Optionally, both the conductive pad and the pads expose the insulating substrate of the flexible circuit board.

Optionally, the flexible circuit board further has a plurality of golden fingers welded with the wire, wherein the golden fingers all expose the insulating substrate of the flexible circuit board. There are n+2 golden fingers, wherein n is an integer greater than 1 and not greater than 8.

Optionally, there are four golden fingers, two temperature sensors, two pairs of pads, and two the third conductive traces.

Optionally, the golden fingers, the conductive pad and the two pairs of pads are located at the same side of the flexible circuit board.

Optionally, the insulated electrode further comprises: a backing, adhered to a corresponding part of the flexible circuit board.

Optionally, the insulated electrode further comprises: an insulating plate, arranged at a side of the flexible circuit board far away from the dielectric element, wherein the insulating plate corresponds to the dielectric element along the thickness direction. The insulating plate is sandwiched between the flexible circuit board and the backing.

Optionally, the insulated electrode comprises: at least one electrode sheet, capable of applying an alternating electric field; and an electrical connector, detachably connected with the electrode sheet. The electrode sheet comprises: an individual electrode unit; and a first wire, electrically connected with the electrode unit, wherein the electrode sheet is detachably connected with the electrical connector through the first wire.

Optionally, a plurality of electrode sheets are connected to the electrical connector in parallel through the corresponding first wires.

Optionally, the first wire of the electrode sheet has a first plug detachably plugged with the electrical connector, wherein the first plug and the electrode unit are located at opposite ends of the first wire respectively.

Optionally, the electrical connector has a plurality of sockets which can be detachably plugged with the first plug of the first wire of the corresponding electrode sheet.

Optionally, the electrical connector is configured with a second wire, wherein the second wire and the plurality of sockets are respectively located at opposite ends of the electrical connector.

Optionally, the second wire has a second plug arranged at an end thereof.

Optionally, the electrical connector has a body, wherein the plurality of sockets and the second wires are respectively arranged at opposite ends of the body.

Optionally, the electrode sheet further comprises: a wiring part, connected with the electrode unit, wherein the wiring part is welded with an end of the first wire away from the first plug.

Optionally, the electrode unit comprises: a main body part; and a dielectric element, welded on one side of the main body part, wherein the wiring part is laterally extended from the main body part.

Optionally, the wiring part and the main body part of the electrode unit form a flexible circuit board of the electrode sheet.

Optionally, the electrode unit further comprises: at least one temperature sensor, wherein the temperature sensor is arranged on the main body part and at the same side as the dielectric element.

Optionally, the middle of the dielectric element is configured with at least one perforation extending therethrough, wherein the temperature sensors are respectively accommodated in the corresponding perforations of the dielectric elements.

Optionally, the electrode unit further comprises an insulating plate adhered to a side of the main body part away from the dielectric element.

Optionally, a periphery of a welding point of the first wire and the wiring part is covered with a heat shrinkable sleeve.

Optionally, the first wire is detachably connected with the electrode unit.

Optionally, the electrode sheet comprises: a wiring part, electrically connected with the electrode unit, wherein an end of the wiring part away from the electrode unit is configured with a docking socket.

Optionally, an end of the first wire away from the first plug is configured with a docking plug, wherein the docking plug is detachably plugged with the docking socket.

Optionally, the electrode sheet further comprises: a backing, adhered to the electrode unit; a support member, arranged around the electrode unit and adhered to the backing; and an adhesive, covering a side of the electrode unit and the support member away from the backing.

Optionally, the insulated electrode is used to apply an electric field to a tumor site of a patient's trunk during the tumor treating, which comprises: a plurality of electrode units, arranged in an array; a plurality of connecting parts, each connected with two adjacent electrode units; and a wire, electrically connected with the plurality of electrode units. There are at least ten electrode units distributed in at least three rows and four columns. Each electrode unit is connected with at least two adjacent electrode units. At least two adjacent electrode units of the plurality of electrode units are arranged in interval rows or interval columns.

Optionally, at least two adjacent electrode units of the plurality of electrode units are arranged in a disconnected state, and a space is formed between the two adjacent electrode units arranged in the disconnected state.

Optionally, the insulated electrode further comprises: a wiring part, electrically connected with the connecting part or the electrode unit, wherein the wiring part passes through the space and is welded with the wire.

Optionally, two adjacent electrode units arranged in rows are arranged in interval columns, and at least two adjacent electrode units arranged in the same column of a plurality of electrode units arranged in columns are arranged in interval rows.

Optionally, the spacings between two adjacent electrode units arranged in rows are the same, and the spacings between two adjacent electrode units arranged in columns are different.

Optionally, a plurality of connecting parts located between two adjacent electrode units in the same row have the same length, and a plurality of connecting parts located between two adjacent electrode units in the same column have different lengths.

Optionally, there are thirteen electrode units distributed in an area arranged in five rows and five columns.

Optionally, at least two adjacent electrode units of a plurality of electrode units arranged in rows are arranged in interval columns, and a plurality of electrode units arranged in columns are all arranged in adjacent rows.

Optionally, the spacings between two adjacent electrode units arranged in rows are different, and the spacings between two adjacent electrode units arranged in columns are the same.

Optionally, a plurality of connecting parts loccated between two adjacent electrode units in the same row have different lengths, and a plurality of connecting parts located between two adjacent electrode units in the same column have the same length.

Optionally, there are thirteen electrode units distributed in an area arranged in three rows and five columns.

Optionally, the connecting part comprises: a first connecting part, connecting two adjacent electrode units located in the same row; and a second connecting part, connecting two adjacent electrode units located in the same column.

Optionally, the connecting part further comprises: a third connecting part, connecting two adjacent electrode units located in adjacent rows and adjacent columns and arranged diagonally.

Optionally, a length of the third connecting part is greater than a length of the first connecting part.

Optionally, a length of the third connecting part is greater than half of the length of the first connecting part.

Optionally, a length of the third connecting part is greater than a length of the second connecting part.

Optionally, the insulated electrode comprises: an electrical functional component, configured to apply an alternating electric field to a patient's trunk. The electrical functional component comprises: a plurality of electrode units, arranged in at least three rows and four columns; a plurality of connecting parts, each connected with two adjacent electrode units; and a wiring part, connected with the connecting parts. Each electrode unit is connected with at least two connecting parts. There are at least 10 electrode units, and numbers of the electrode units in each row or column are not exactly the same.

Optionally, there are twenty electrode units distributed in an array area of four rows and six columns.

Optionally, at least two adjacent electrode units of the plurality of electrode units in the same row or the same column are arranged in a disconnected state.

Optionally, the electrical functional component has a space formed between the two adjacent electrode units arranged in the disconnected state. The wiring part passes through the space.

Optionally, the wiring part is arranged by a connecting part extending in a direction toward the space.

Optionally, the wiring part and the connecting part are vertically arranged, wherein the wiring part is substantially arranged in a shape of the Chinese character

Optionally, the wiring part is bridged between two connecting parts respectively connected with two adjacent electrode units arranged in the disconnected state.

Optionally, the wiring part is substantially in a “T” shape.

Optionally, the spacings between two adjacent electrode units arranged in rows are the same, and the plurality of connecting parts each connected with the two adjacent electrode units arranged in rows have the same length.

Optionally, the spacings between two adjacent electrode units arranged in columns are the same, and the plurality of connecting parts each connected with the two adjacent electrode units arranged in columns have the same length.

Optionally, among the plurality of electrode units, at least two adjacent electrode units arranged in rows are arranged in interval columns, and the spacings between two adjacent electrode units arranged in rows are not exactly the same.

Optionally, among the plurality of electrode units, at least two adjacent electrode units arranged in columns are arranged in interval rows, and the spacings between two adjacent electrode units arranged in columns are not exactly the same.

Optionally, two adjacent electrode units arranged in rows are arranged in adjacent columns, wherein the spacings between two adjacent electrode units arranged in rows are the same.

Optionally, two adjacent electrode units arranged in columns are arranged in adjacent rows, wherein the spacings between two adjacent electrode units arranged in columns are the same.

Optionally, the spacings between two adjacent electrode units arranged in rows are the same, and the spacings between two adjacent electrode units arranged in columns are the same.

Optionally, the plurality of electrode units are distributed in an array area of four rows and six columns in a manner that: two electrode units are arranged in each column of the first column and the last column, and four electrode units are arranged in each of the four middle columns.

Optionally, the plurality of electrode units are arranged axial-symmetrically in a row direction and are arranged axial-symmetrically in a column direction.

Optionally, the insulated electrode further comprises: a wire, electrically connected with electrical functional components, wherein the wire is welded with the wiring part.

Optionally, the insulated electrode further comprises: a backing, configured for supporting the electrical functional components, wherein the backing is configured with a threading hole for the wire to pass through.

Optionally, the insulated electrode comprises: an electrical functional component, configured to apply an alternating electric field to a patient's tumor site; and a wire, electrically connected with the electrical functional component. The electrical functional component comprises: a plurality of electrode units, arranged at intervals; a plurality of connecting parts, each connected with two adjacent electrode units; and a wiring part, electrically connected with the wire. Each electrode unit is connected with at least two connecting parts. There are at least ten electrode units.

Optionally, each electrode unit is connected with at least two electrode units adjacent thereto.

Optionally, the plurality of electrode units are distributed in an array area of at least three rows and four columns. A number of the electrode units is at least 10 and at most 30.

Optionally, the plurality of electrode units are distributed in an array area of at least three rows and four columns. A number of the electrode units in each row is the same, and the electrode units in each row are arranged in column-aligned manner.

Optionally, the electrode units are arranged with the same row spacing.

Optionally, the electrode units are arranged with the same column spacing.

Optionally, the plurality of connecting parts each connected with the two adjacent electrode units arranged in rows have the same length.

Optionally, the plurality of connecting parts each connected with the two adjacent electrode units arranged in columns have the same length.

Optionally, at least two adjacent electrode units of the plurality of electrode units in the same row or the same column are arranged in a disconnected state. A space is formed between the two adjacent electrode units arranged in a disconnected state, wherein the space is configured for the wiring part to pass through.

Optionally, the wiring part is laterally extended from the connecting part opposite to the space.

Optionally, the connecting part from which the wiring part extends is vertically arranged with the wiring part.

Optionally, the wiring part is bridged between two connecting parts respectively connected with two electrode units arranged in the disconnected state.

Optionally, the wiring part is substantially in a “T” shape.

Optionally, the plurality of electrode units are arranged in an array of four rows and five columns, and a number of the electrode units is 20.

Optionally, the electrode unit comprises: a main body part, arranged at an end of the connecting part; an insulating plate, arranged at a side of the main body part away from human skin; and a dielectric element; arranged at a side of the main body part facing human skin.

Optionally, the electrode unit further comprises: a temperature sensor, selectively arranged on the main body part, wherein the temperature sensor and the dielectric element are located at the same side of the main body part.

Optionally, the dielectric element is configured with a perforation corresponding to the temperature sensor.

Optionally, the insulated electrode further comprises: a backing, configured for supporting the electrical functional components, wherein the backing is configured with a threading hole for the wire to pass through.

Optionally, the wire is configured with a heat shrinkable sleeve covering a joint between the wire and the wiring part.

Optionally, the insulated electrode comprises: an electrical functional component; a backing, adhered to corresponding parts of the electrical functional component; and a wire, electrically connected with the electrical functional component. The electrical functional component comprises: at least nine electrode units, arranged in an array; and a plurality of connecting parts each located between two adjacent electrode units. The electrode unit comprises: at least one central electrode unit; and a plurality of peripheral electrode units, located on the periphery of the central electrode unit. The electrical functional component further comprises: a plurality of temperature sensors, selectively arranged on the corresponding peripheral electrode units.

Optionally, a number of the peripheral electrode units is at least 8, and a number of the temperature sensors is no more than 8.

Optionally, the number of the temperature sensors is 8. The electrode units are distributed in an array area of at least three rows and three columns.

Optionally, there are 9 electrode units distributed in an area arranged in three rows and three columns. The center electrode unit is an electrode unit located at an overlapping position of the middle row and the middle column. The peripheral electrode units are electrode units located at the remaining positions in the array.

Optionally, each of the electrode units is connected to at least two adjacent electrode units through corresponding connecting parts.

Optionally, there are 13 electrode units distributed in an area arranged in five rows and five columns. The center electrode unit is an electrode unit located at an overlapping position of the middle row and the middle column. The peripheral electrode units are electrode units located at the remaining positions in the array.

Optionally, the electrical functional component comprises: a wiring part, welded to the wire, wherein the wiring part is laterally extended from the peripheral electrode unit.

Optionally, the 13 electrode units are arranged in a manner that: two electrode units are arranged in each row of the first row and the last row, and three electrode units are arranged in each of the three middle rows. The two adjacent electrode units in each row are arranged in interval columns, and the electrode units located in the first row and the last row are respectively arranged in staggered columns with the electrode units in adjacent rows.

Optionally, two adjacent electrode units in the last row are arranged in a disconnected state, and form a space for the wiring part to pass through.

Optionally, the wiring part is laterally extended from the electrode unit located in the middle column of the fourth row.

Optionally, there are 20 electrode units distributed in an area arranged in four rows and six columns. The center electrode units are the four electrode units located at an overlapping position of the middle two rows and the middle column. The peripheral electrode units are electrode units located at the remaining positions in the array.

Optionally, the 20 electrode units are arranged in a manner that: four electrode units are arranged in each row of the first row and the last row, and six electrode units are arranged in each of the two middle rows. The electrode units in the first row and the last row are arranged in column-aligned manner, and are all located in the middle four columns.

Optionally, the electrical functional component comprises: a wiring part, welded to the wire, wherein the wiring part is bridged between two connecting parts located between the four center electrode units.

Optionally, at least two adjacent center electrode units of the four center electrode units are arranged in a disconnected state, and a space is formed between the two center electrode units arranged in the disconnected state. The wiring part passes through the space.

Optionally, the wiring part is provided as a “T” shape.

Optionally, each the electrode unit comprises: a main body part, arranged at an end of the connecting part; and a dielectric element; arranged at a side of the main body part facing human skin. The temperature sensor is arranged on the main body part of the corresponding peripheral electrode unit, and is located at the same side as the dielectric component.

Optionally, the dielectric element is configured with perforations, and the temperature sensor is accommodated in the perforation of the corresponding dielectric element.

The electric field generator of the tumor treating fields system of the present application may monitor, via the signal controller, temperature information corresponding to the obtained temperature signals based on the insulated electrodes, and individually control the output of the alternating current signals, which ensures that the insulated electrode is at a safety threshold and improves the usage efficiency of the electric field.

With reference to the embodiments described hereinafter, these and other aspects of the present disclosure will be apparent, and will be set forth with reference to the embodiments described hereinafter.

### BRIEF DESCRIPTION OF THE DRAWINGS

Further details, features and advantages of the present disclosure are disclosed in the following description of example embodiments in conjunction with the accompanying drawings, in which:

FIG. 1 illustrates a block diagram of a tumor treating fields system of the present application.

FIG. 2 is a schematic diagram of control signals for turning on or off a first electric field and a second electric field in a tumor treating fields system.

FIG. 3 shows a relationship between cell growth rate and electric field work cycle.

FIG. 4 is a schematic diagram of an AC signal applied to an insulated electrode of a tumor treating fields system.

FIG. 5 is a three-dimensional assembled view of a first embodiment of an insulated electrode of a tumor treating fields system according to the present application.

FIG. 6 is another three-dimensional assembled view of the insulated electrode shown in FIG. 5, in which the release paper is not shown.

FIG. 7 is a three-dimensional exploded view of the insulated electrode shown in FIG. 6.

FIG. 8 is a plan view of an electrical functional component of the insulated electrode shown in FIG. 7.

FIG. 9 is a three-dimensional exploded view of a wire and the electrical functional component of the insulated electrode shown in FIG. 7.

FIG. 10 is a three-dimensional view of a dielectric element of the electrical functional component shown in FIG. 9.

FIG. 11 is a sectional view taken along the A-A direction of the electrical functional component shown in FIG. 7.

FIG. 12 is a front wiring diagram of a flexible circuit board of the electrical functional component shown in FIG. 9.

FIG. 13 is a back wiring diagram of a flexible circuit board of the electrical functional component shown in FIG. 9.

FIG. 14 is an alternate implementation of the first embodiment of the insulated electrode in FIG. 5, in which the adhesive and the release paper are not shown.

FIG. 15 is a three-dimensional assembled view of a second embodiment of an insulated electrode of a tumor treating fields system of the present application.

FIG. 16 is a top view of the insulated electrode shown in FIG. 15.

FIG. 17 is a partial three-dimensional exploded view of the insulated electrode shown in FIG. 16.

FIG. 18 is a top view of the electrical functional component shown in FIG. 17.

FIG. 19 is a three-dimensional exploded view of a wire and the electrical functional component of the insulated electrode shown in FIG. 17.

FIG. 20 is a three-dimensional assembled view of a third embodiment of an insulated electrode of a tumor treating fields system of the present application.

FIG. 21 is a partial three-dimensional exploded view of the insulated electrode shown in FIG. 20.

FIG. 22 is a three-dimensional exploded view of a wire and the electrical functional component of the insulated electrode shown in FIG. 21.

FIG. 23 is a front wiring diagram of a flexible circuit board of the electrical functional component shown in FIG. 22.

FIG. 24 is a back wiring diagram of a flexible circuit board of the electrical functional component shown in FIG. 22.

FIG. 25 is a three-dimensional assembled view of a forth embodiment of an insulated electrode of a tumor treating fields system of the present application.

FIG. 26 is a partial three-dimensional exploded view of the insulated electrode shown in FIG. 25.

FIG. 27 is a three-dimensional exploded view of a wire and the electrical functional component of the insulated electrode shown in FIG. 26.

FIG. 28 is a plane schematic diagram of a flexible circuit board of the insulated electrode shown in FIG. 27.

FIG. 29 is a front wiring diagram of a flexible circuit board of the electrical functional component shown in FIG. 28.

FIG. 30 is a back wiring diagram of a flexible circuit board of the electrical functional component shown in FIG. 29.

FIG. 31 is similar to FIG. 25, in which a backing of the insulated electrode adopts an alternate implementation.

FIG. 32 is a partial three-dimensional exploded view of a fifth embodiment of an insulated electrode of a tumor treating fields system of the present application.

FIG. 33 is a three-dimensional exploded view of a wire and the electrical functional component of the insulated electrode shown in FIG. 31.

FIG. 34 is a plane schematic diagram of a flexible circuit board of the insulated electrode shown in FIG. 31.

FIG. 35 is a three-dimensional assembled view of a sixth embodiment of an insulated electrode of a tumor treating fields system of the present application.

FIG. 36 is an exploded view of an electrical connector and an electrode sheet of the insulated electrode shown in FIG. 35.

FIG. 37 is an alternate implementation of the sixth embodiment of an insulated electrode of a tumor treating fields system of the present application.

FIG. 38 is a three-dimensional assembled view of a seventh embodiment of an insulated electrode of a tumor treating fields system of the present application.

FIG. 39 is a partial three-dimensional exploded view of the insulated electrode shown in FIG. 38.

FIG. 40 is a three-dimensional exploded view of a wire and the electrical functional component of the insulated electrode shown in FIG. 39.

FIG. 41 is a plan view of a flexible circuit board of the electrical functional component shown in FIG. 40.

FIG. 42 is a plan view of the electrical functional component shown in FIG. 39.

FIG. 43 is a three-dimensional assembled view of an alternate implementation of the seventh embodiment of an insulated electrode of a tumor treating fields system of the present application.

FIG. 44 is a three-dimensional exploded view of the electrical functional component of the insulated electrode shown in FIG. 43.

FIG. 45 is a three-dimensional assembled view of another alternate implementation of the seventh embodiment of an insulated electrode of a tumor treating fields system of the present application.

FIG. 46 is a three-dimensional view of a flexible circuit board of the insulated electrode shown in FIG. 45.

FIG. 47 is a three-dimensional assembled view of an eighth embodiment of an insulated electrode of a tumor treating fields system of the present application.

FIG. 48 is a plane view of the back of the insulated electrode shown in FIG. 47.

FIG. 49 is a partial three-dimensional exploded view of the insulated electrode shown in FIG. 47.

FIG. 50 is a three-dimensional exploded view of a wire and the electrical functional component of the insulated electrode shown in FIG. 49.

FIG. 51 is a plan view of an electrical functional component of the insulated electrode shown in FIG. 49.

FIG. 52 is a plan view of an alternate embodiment of the eighth embodiment of an insulated electrode of a tumor treating fields system of the present application.

FIG. 53 is a partial three-dimensional exploded view of the insulated electrode shown in FIG. 52.

FIG. 54 is a plan view of an electrical functional component of the insulated electrode shown in FIG. 53.

FIG. 55 is a three-dimensional exploded schematic diagram of a ninth embodiment of an insulated electrode of a tumor treating fields system of the present application.

FIG. 56 is a three-dimensional exploded schematic diagram of a temperature sensor, a dielectric element, a flexible circuit board and a heat dissipation reinforcement of the insulated electrode shown in FIG. 55.

FIG. 57 is a three-dimensional schematic diagram of a heat dissipation reinforcement of the insulated electrode shown in FIG. 56.

FIG. 58 is similar to FIG. 55, which is a partially three-dimensional exploded schematic diagram of an insulated electrode.

FIG. 59 is a three-dimensional exploded schematic diagram of an alternate embodiment of the ninth embodiment of an insulated electrode of a tumor treating fields system of the present application.

FIG. 60 is a three-dimensional exploded view of an electrical functional component of the insulated electrode shown in FIG. 59.

FIG. 61 is a structural schematic diagram of a flexible circuit board of the electrical functional component shown in FIG. 60.

FIG. 62 is a structural schematic diagram of a semiconductor refrigerator of the insulated electrodes shown in FIG. 61.

FIG. 63 is a sectional view of the semiconductor refrigerator and flexible circuit board shown in FIG. 62.

FIG. 64 is a flowchart of the steps of temperature control of the tumor treating fields system with the insulated electrode shown in FIG. 59.

FIG. 65 is a schematic flowchart of temperature control of the tumor treating fields system with the insulated electrode shown in FIG. 59.

FIG. 66 is a schematic block diagram of an electric field generator of the tumor treating fields system of the present application.

FIG. 67 is another schematic block diagram of the electric field generator shown in FIG. 66.

FIG. 68 is a structural schematic block diagram of a tumor treating fields system with the electric field generator shown in FIG. 66 or FIG. 67.

FIG. 69 is a flowchart of a method of the tumor treating fields system shown in FIG. 68 applying alternating electrical signals.

FIG. 70 is a flowchart of the control of applying alternating electrical signals in the step 2 shown in FIG. 69.

FIG. 71 is a further flowchart of the control of applying alternating electrical signals in the step 2 shown in FIG. 69.

FIG. 72 is a flowchart of the operation of the tumor treating fields system shown in FIG. 68 applying alternating electrical signals.

FIG. 73 is another schematic block diagram of the tumor treating fields system shown in FIG. 68.

FIG. 74 is a schematic block diagram of a converter of the tumor treating fields system shown in FIG. 73.

FIG. 75 is a schematic circuit diagram of the converter shown in FIG. 73.

### DETAILED DESCRIPTION OF EMBODIMENTS

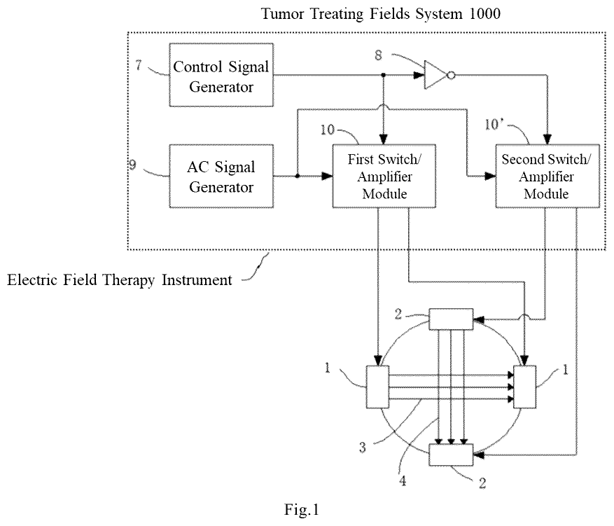

FIG. 1 illustrates a block diagram of an embodiment of a tumor treating fields system 1000 of the present application. The tumor treating fields system 1000 includes: a first pair of insulated electrodes 1, a second pair of insulated electrodes 2, and an electric field therapy instrument (not labeled) electrically connected with the first pair of insulated electrodes 1 and the second pair of insulated electrodes 2. The electric field therapy instrument (not labeled) applies alternating electrical signals for tumor treatment to the first pair of insulated electrodes 1 and the second pair of insulated electrodes 2. The electric field therapy instrument (not labeled) includes: an electric field generator (not shown), and an adapter (not shown) electrically connected with the electric field generator (not shown). The first pair of insulated electrodes 1 and the second pair of insulated electrodes 2 may be directly electrically connected with the electric field generator (not shown), or may be first electrically connected with the adapter (not shown), and then electrically connected with the electric field generator (not shown) through the adapter (not shown).

The electric field therapy instrument (not labeled) includes: a control signal generator 7; an inverter 8 electrically connected with the control signal generator 7; an alternating current signal generator 9; a first switch/amplifier module 10 electrically connected with both the alternating current signal generator 9 and the control signal generator 7; and a second switch/amplifier module 10′ electrically connected with both the alternating current signal generator 9 and the inverter 8. In one embodiment, the control signal generator 7, the inverter 8, the alternating current signal generator 9, the first switch/amplifier module 10 and the second switch/amplifier module 10′ may all be arranged in the electric field generator (not shown) electrically connected with the first pair of insulated electrodes 1 and the second pair of insulated electrodes 2. In another embodiment, the control signal generator 7, the inverter 8 and the alternating current signal generator 9 are arranged in the electric field generator (not shown), and the first switch/amplifier module 10 and the second switch/amplifier module 10′ are both arranged in the adapter (not shown).

In one embodiment, the first switch/amplifier module 10 may be divided into two elements: a first switch arranged in the adapter (not shown), and an amplifier arranged in the electric field generator (not shown). The second switch/amplifier module 10′ may also be divided into two elements: a second switch and an amplifier, and the second switch is arranged in the adapter and the amplifier is arranged in the electric field generator (not shown). In another embodiment, the alternating current signal generator 9, the control signal generator 7, the inverter 8 and the amplifier are all arranged in the electric field generator (not shown), and the first switch and the second switch are both arranged in the adapter (not shown).

The alternating current signal generator 9 is used to output sinusoidal signals with adjustable frequency and amplitude. In the present embodiment, the control signal generator 7 is a square wave generator generating square wave signals, and the inverter 8 is used to invert square wave signals from the control signal generator 7. A control terminal of the first switch/amplifier module 10 is directly connected with the control signal generator 7, and a control terminal of the second switch/amplifier module 10′ is connected with the control signal generator 7 through the inverter 8. Input terminals of the first switch/amplifier module 10 and the second switch/amplifier module 10′ are both connected with the alternating current signal generator 9. An output terminal of the first switch/amplifier module 10 is connected to the first pair of insulated electrodes 1, and an output terminal of the second switch/amplifier module 10′ is connected to the second pair of insulated electrodes 2. The first switch/amplifier module 10 and the second switch/amplifier module 10′ have the function of signal amplification, and also serve as switches. The control signal generator 7 controls turning on of the first switch/amplifier module 10 and the second switch/amplifier module 10′, so that AC signals generated by the alternating current signal generator 9 are applied to the first pair of insulated electrodes 1 and the second pair of insulated electrodes 2.

When the first pair of insulated electrodes 1 are turned on, a first electric field 3 in a first direction is generated, and when the second pair of insulated electrodes 2 are turned on, a second electric field 4 in a second direction is generated. The first pair of insulated electrodes 1 and the second pair of insulated electrodes 2 are arranged in such a way that the electric field directions of the first electric field 3 and the second electric field 4 are perpendicular to each other. Each insulated electrode in the first pair of insulated electrodes 1 and the second pair of insulated electrodes 2 includes an electrical functional component and a backing that supports the electrical functional component. Preferably, the backing has an adhesive layer. The adhesive layer is attached to a patient's head to place the electrical functional component on the surface of the patient's head. The first pair of insulated electrodes 1 and the second pair of insulated electrodes 2 are controlled to be alternately turned on to form an alternating treating electric field acting on a target area, that is, the first electric field 3 and the second electric field 4 that applied alternately.

As one embodiment, the alternating current signal generator 9 generates an intermediate frequency alternating current signal of 200 KHz. The control signal generator 7 outputs a square wave having a first output state and a second output state, i.e., high level 1 and low level 0. In another embodiment, the alternating current signal generator 9 may also generate an intermediate frequency alternating current signal of 150 KHz.

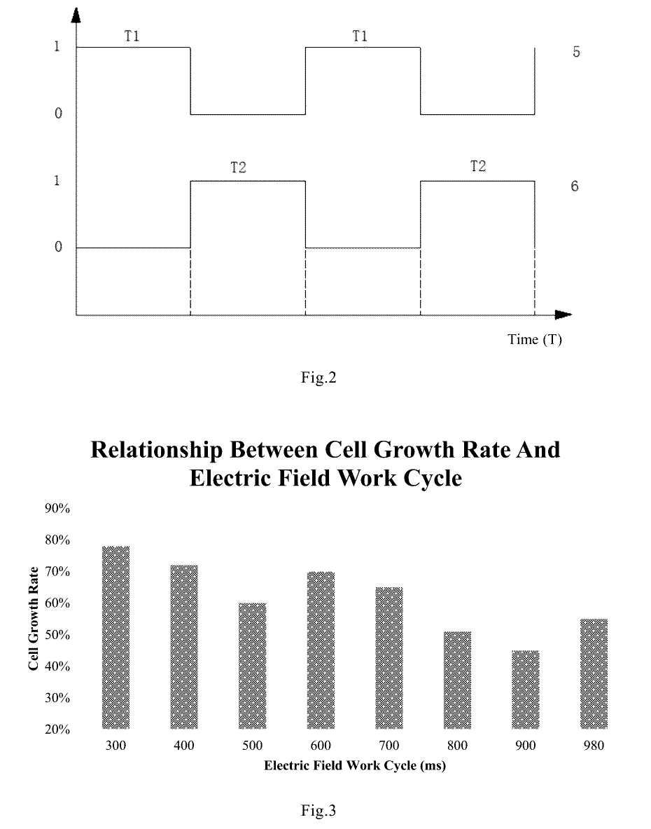

FIG. 2 is a schematic diagram of control signals for turning on or off the first electric field 3 and the second electric field 4 in the tumor treating fields system shown in FIG. 1. A control signal input to the first switch/amplifier module 10 by the control signal generator 7 is similar to the signal 5 in FIG. 2 used to turn on or off the first electric field 3. Due to the arrangement of the inverter 8, a signal received by the second switch/amplifier module 10′ is similar to the signal 6 in FIG. 2 used to turn on or off the second electric field 4. The alternating current signal generator 9 generating an intermediate frequency alternating current signal of 200 KHz is taken as an example for illustration.

During the T1 period, when the control signal generator 7 outputs a control signal in a first output state, the first switch/amplifier module 10 is turned on and controls the turning-on of the AC signal to the first pair of insulated electrodes 1. A first AC signal with a frequency of 200 KHz is generated between conductors of the first pair of insulated electrodes 1. A first electric field 3 with an intensity of at least TV/cm is generated in a target induction area. Meanwhile, the AC signal to the second pair of insulated electrodes 2 is turned off, and the second electric field 4 is turned off. At this point, the signal 5 is at the high level 1, and the signal 6 is at the low level 0.

During the T2 period, when the control signal generator 7 outputs a control signal in a second output state, the second switch/amplifier module 10′ is turned on and controls the turning-on of the AC signal to the second pair of insulated electrodes 2. A second AC signal with a frequency of 200 KHz is generated between conductors of the second pair of insulated electrodes 2. A second electric field 4 with an intensity of at least 1V/cm is generated in the target induction area. Meanwhile, the AC signal to the first pair of insulated electrodes 1 is turned off, and the first electric field 3 is turned off. At this point, the signal 5 is at the low level 0, and the signal 6 is at the high level 1.

The T1 period is a duration time of the control signal of the control signal generator 7 in the first output state, is a continuous conducting duration of the first electric field 3 in each work cycle, and is also a turn-off duration of the second electric field 4. The T2 period is a duration time of the control signal of the control signal generator 7 in the second output state, is a continuous conducting duration of the second electric field 4 in each work cycle, and is also a turn-off duration of the first electric field 3. In the present embodiment, T1 and T2 are the same that T1 and T2 each occupies half of the work cycle of the control signal of the control signal generator 7.

By controlling the first switch/amplifier module 10 and the second switch/amplifier module 10′, the control signal generator 7 may switch the 200 KHz intermediate frequency AC signal generated by the alternating current signal generator 9 between the first pair of insulated electrodes 1 and the second pair of insulated electrodes 2, so that the first electric field 3 and the second electric field 4 are alternately applied to the target induction area.

FIG. 3 shows an effect of applying electric fields with different work cycles on cell proliferation during glioma cell culture. The applied electric fields switch at different rates between different directions, and thus the inhibitory effects of tumor treating fields on proliferating cells in tissue culture and malignant cells in experimental animals are different.

In the experiment, glioma cells were cultured in a petri dish, and two pairs of mutually perpendicular alternating current signals of 200 KHz were applied around the glioma cells. The proliferation of the cells was observed by changing the switching rate of the first electric field 3 and the second electric field 4. As shown in FIG. 3, the first electric field 3 is switched to the second electric field 4 after working for T1, and the second electric field 4 is switched to the first electric field 3 after working for T2, and so on. T1 and T2 are the same that each is half of a cycle of the control signal of the control signal generator 7. The experimental results show that, when T1 and T2 are within a range from 400 ms to 980 ms, the effects of inhibiting cell proliferation are superior to other rates. Preferably, when T1 and T2 are about 500 ms or between 700 ms and 980 ms, the effects of inhibiting cell proliferation are much better. In the present embodiment, U87MG glioma is used as cell tissue for culture. However, the effect of switching rates on inhibiting cell proliferation is not limited to that cell. Other rapidly proliferating cells may also be suitable.

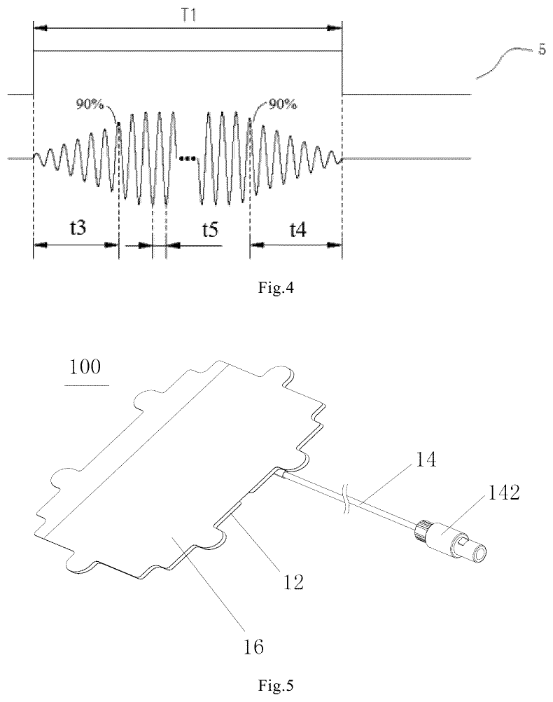

Since there are non-pure resistive devices in the system, the voltage spike caused by the non-pure resistive devices needs to be suppressed for biological applications. In addition to using insulated electrodes as barriers, preferably, such a phenomenon can be effectively avoided by controlling the climbing rate of the AC signal generated by the alternating current signal generator 9 when it is turned on and off. FIG. 4 illustrates an AC signal applied to the first pair of insulated electrodes 1, in which the climbing rate of the AC signal at the time of turning on and off has been optimized.

During the T1 period, the alternating current signal generator 9 applies the first AC signal to the first pair of insulated electrodes 1, and generates the first electric field 3. In the initial process of the formation of the first AC signal, the voltage is increased in a step-up 5 manner. That is, during the switch-to-turn-on period t3, the AC voltage amplitude of the first AC signal applied to the first pair of insulated electrodes 1 is gradually increased from 0V to a specific value in the step-up manner. The specific value is 90% of a peak value of the target voltage amplitude. The peak value of the target voltage amplitude is a peak value of the output AC voltage amplitude set by the electric field generator (not shown). The first AC signal also 10 has several stable-output-AC-voltage periods t5 between the switch-to-turn-on period t3 and the switch-to-turn-off period t4. During the stable-output-AC-voltage period t5, the AC voltage value of the first AC signal applied to the first pair of insulated electrodes 1 is between the specific value and the peak value of the output AC voltage amplitude set by the electric field generator (not shown). During the switch-to-turn-off period t4, the AC voltage of the first AC signal applied to the first pair of insulated electrodes 1 is gradually and slowly reduced from the specific value to 0V in a step-down manner. Similarly, during the T2 period, the alternating current signal generator 9 applies the second AC signal to the second pair of insulated electrodes 2, and generates the second electric field 4. In the initial process of the formation of the second AC signal, the voltage is increased in a step-up manner. That is, during the switch-to-turn-on period t3, the AC voltage amplitude of the second AC signal applied to the second pair of insulated electrodes 2 is gradually increased from 0V to a specific value in the step-up manner. The specific value is 90% of a peak value of the target voltage amplitude. The peak value of the target voltage amplitude is a peak value of the output AC voltage amplitude set by the electric field generator (not shown). The second AC signal also has several stable-output-AC-voltage periods t5 between the switch-to-turn-on period t3 and the switch-to-turn-off period t4. During the stable-output-AC-voltage period t5, the AC voltage value applied to the second pair of insulated electrodes 2 is between the specific value and the peak value of the output AC voltage amplitude set by the electric field generator (not shown). During the switch-to-turn-off period t4, the AC voltage of the second AC signal applied to the second pair of insulated electrodes 2 is gradually and slowly reduced from the specific value to 0V. Switching the first AC signal applied to the first pair of insulated electrodes 1 to be turned on when the AC voltage of the second AC signal applied to the second pair of insulated electrodes 2 is reduced to 0V may effectively avoid the problem that the alternating current signal generator 9 applies voltage to the first pair of insulated electrodes 1 and the second pair of insulated electrodes 2 at the same time if the AC voltage applied to the second pair of insulated electrodes 2 is switched without being reduced to 0V when the AC signal to the second pair of insulated electrodes 2 is cut off. Therefore, the situation where the first electric field 3 and the second electric field 4 coexist and overlap is avoided.

The switch-to-turn-on period t3 and the switch-to-turn-off period t4 are usually within 10% of the duration of T1 or T2, so as to avoid the peak signal generated by a sudden change of alternating electrical signals when switching to turn-on or switching to turn-off impact and damage the control signal generator 7 or other electronic components. At the same time, the time reaching the electric field intensity for tumor treatment may be ensured as long as possible during a work cycle, so as to ensure the effect of tumor treating fields therapy. In the present embodiment, the sum of the switch-to-turn-on period t3, the switch-to-turn-off period t4, and the several stable-output-AC-voltage periods t5 of the alternating electrical signal is equal to the working period T1 or T2 in each cycle of the alternating electrical signal. During the T1 period, the first electric field 3 between the first pair of insulated electrodes 1 is turned on, and the second electric field 4 between the second pair of insulated electrodes 2 is turned off; while during the T2 period, the first electric field 3 between the first pair of insulated electrodes 1 is turned off, and the second electric field 4 between the second pair of insulated electrodes 2 is turned on, thereby completing a cyclic switching.

Based on the above description, the alternating current signal generator 9 of the tumor treating fields system 1000 of the present application generates a specific intermediate frequency alternating current signal of 200 KHz or 150 KHz. By forming two electric fields with an intensity of 1V/cm that are in two mutually perpendicular directions and alternately applied to the target sensing area through the two pairs of insulated electrodes 1 and 2, and by switching between the first output state and the second output state, so that it is switched between the first AC signal generated between the first pair of insulated electrodes 1 and the second AC signal generated between the second pair of insulated electrodes 2, and the duration of the first output state and the second output state is between 400 ms and 980 ms, a better effect of inhibiting tumor cell proliferation may be achieved.

The four insulated electrodes of the first pair of insulated electrodes 1 and the second pair of insulated electrodes 2 in the tumor treating fields system 1000 of the present embodiment have the same structure. The insulate electrodes of the present application may have different implementations. The insulated electrodes of the present application provide various implementations as follows.

### A First Embodiment of the Insulated Electrode

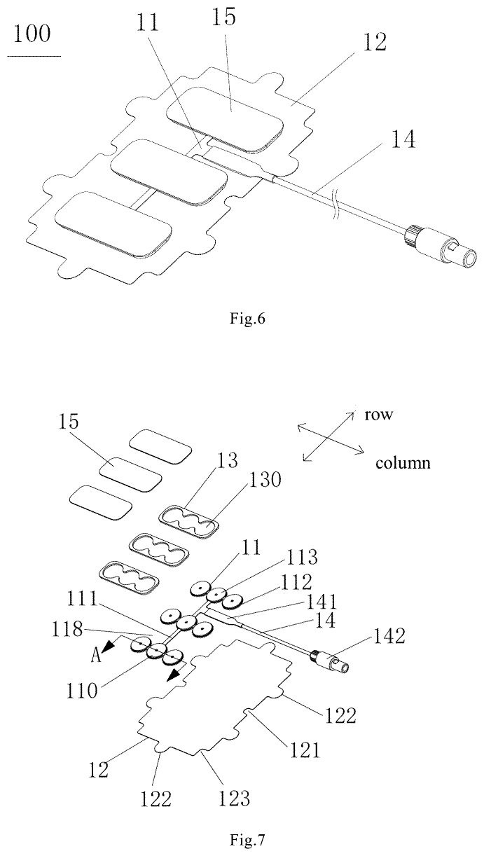

FIG. 5 to FIG. 13 illustrate the first embodiment of the insulated electrode 100 of the present application, which includes: a backing 12; an electrical functional component 11 adhered to the backing 12; support members 13 adhered to the backing 12; a wire 14 electrically connected to the electrical functional component 11; and an adhesive 15 covering the corresponding parts of the support members 13 and the electrical functional component 11. The insulated electrode 100 is attached to the body surface corresponding to a patient's tumor site through the backing 12, and an alternating electric field is applied to the patient's tumor site through the electrical functional component 11 to interfere or prevent the mitosis of the patient's tumor cells, so as to achieve the purpose of treating the tumor.

The backing 12 is in a sheet shape, which is mainly made of a flexible and permeable insulating material. The backing 12 is a mesh fabric. Specifically, the backing 12 is a mesh non-woven fabric, which has the characteristics of softness, lightness, moisture-proof and permeability, and can keep the patient's skin surface dry after being attached to the patient's body surface for a long time. The side of the backing 12 facing the patient's body surface is also coated with a biocompatible adhesive (not shown) for tightly adhering the backing 12 to the body surface corresponding to the patient's tumor site.

In the present embodiment, the backing 12 is substantially in a cuboid sheet shape. Edges of the backing 12 are in concave-convex shapes. The backing 12 has two notches 121 recessed inward from centers of long sides of the backing 12. When applyed to a surface of the patient's body, the notches 121 are aligned with upper edges of the patient's external auditory canal bone. The backing 12 also has concave corners 123 recessed inward from four corners thereof, so as to avoid the formation of wrinkles when the backing 12 is attached to part of the body surface corresponding to the tumor, and further avoid air entering between the adhesive 15 and the skin from the wrinkles, which results in cryogenic burns due to the increase of heat generation of the electrical functional component 11 causing by the increase of the impedance between the electrical functional component 11 and the skin. The concave corners 123 communicate with the outside, and are in an “L” shape. An angle between two sides of the backing 12 that forms a concave corner 123 is greater than or equal to 90 degrees. The backing 12 also has a plurality of lateral wings 122 extending outward from the peripheral side thereof. The plurality of lateral wings 122 may be held by an operator to apply the insulated electrode 100 to the body surface of the part corresponding to the patient's tumor. Two lateral wings 122 located on a long side of the backing 12 are symmetrically arranged on the same long side at two sides of the notch 121. The lateral wings 122 located on short sides of the backing 12 are arranged at centers of the short sides, corresponding to the position of the patient's eyebrow or occipital bone, so as to assist applying the insulated electrode 100 to the body surface corresponding to the patient's tumor site. The lateral wings 122 are axial symmetrically arranged on the peripheral side of the backing 12.

The electrical functional component 11 includes: a plurality of electrode units 110 arranged in an array; a plurality of connecting parts 1112 each connected with two adjacent electrode units 110; and a wiring part 1113 extending laterally from a connecting part 1112. The electrode units 110 are arranged at intervals that forms open spaces 118 between the electrode units 110, so as to allow the skin of the body surface corresponding to the patient's tumor site covered by the insulated electrodes 100 to breathe freely after configuring the insulated electrodes 100 at the body surface corresponding to the patient's tumor site. The wiring part 1113 is laterally extended from the connecting part 1112, and is partially located in the open space 118, so as to shorten the distance of the wiring part 1113 beyond the edge of the electrical functional component 11, resulting in a more compact arrangement of the electrical functional component 11, and avoiding the increase in manufacturing cost caused by increasing the overall size of the electrical functional component 11. The wiring part 1113 is located between two adjacent columns of electrode units 110. A width of the wiring part 1113 is at least 4 mm. Preferably, a width of the wiring part 1113 is 4 mm to 8 mm. A spacing between the wiring part 1113 and the electrode unit 110 adjacent to the wiring part 1113 is at least 2 mm. That is, a column spacing between the two columns of electrode units 110 located at the two sides of the wiring part 1113 is at least 8 mm.

The electrode units 110 of the electrical functional component 11 may have the same column spacing or different column spacing. The column spacing between two adjacent columns of the electrode units 110 of an electrical functional component 11 without a wiring part 1113 is at least 1 mm. The electrode units 110 of the electrical functional component 11 may have the same row spacing or different row spacing. The row spacing of the electrode units 110 of the electrical functional component 11 is at least 1 mm. Preferably, the electrode units 110 of the electrical functional component 11 have the same column spacing and the same row spacing, while the column spacing is different from the row spacing. Preferably, the column spacing of these electrode units 110 of the electrical functional component 11 is larger than the row spacing. That is, a spacing between electrode units 110 in adjacent rows is smaller than a spacing between electrode units 110 in adjacent columns. That is, the column spacing of the electrical units 110 of the electrical functional component 11 is at least 8 mm, and the row spacing of the electrical units 110 of the electrical functional component 11 is at least 1 mm. The spacing between two adjacent columns of electrode units 110 is at least 8 mm, and the spacing between two adjacent rows of electrode units 110 is at least 1 mm.

The electrode unit 110 has a substantially circular sheet structure. A diameter of the electrode unit 110 is at least 21 mm. Preferably, the diameter of the electrode unit 110 is 21 mm to 22 mm. A width of the connecting part 1112 is 4.5 mm to 6 mm, which may be determined according to the manufacturing cost and wiring requirements of making enough open spaces 118 between the electrode units 110 to facilitate heat and water vapor to escape. Preferably, the width of the connecting part 1112 is 4.5 mm. A length of the connecting part 1112 is close to the distance between the two electrode units 110 connected by the connecting part 1112. The dimension of the length of the connecting part 1112 may overlap with edges of the electrode unit 110 to a certain extent, so that the dimension of the length of the connecting part 1112 is slightly larger than the distance between the two electrode units 110 connected by the connecting part 1112.

The connecting parts 1112 each connecting two adjacent electrode units 110 in the same column have the same length. The connecting parts 1112 each connecting two adjacent electrode units 110 in the same row have the same length. The length of the connecting parts 1112 each connecting two adjacent electrode units 110 in the same column is different from the length of the connecting parts 1112 each connecting two adjacent electrode units 110 in the same row. The length of the connecting parts 1112 each connecting two adjacent electrode units 110 in the same row is larger than the length of the connecting parts 1112 each connecting two adjacent electrode units 110 in the same column. Specifically, the connecting parts 1112 includes: first connecting parts 11120 each connecting two adjacent electrode units 110 in the same column; and second connecting parts 11121 each connecting two adjacent electrode units 110 in the same row. The length of a first connecting part 11120 is smaller than the length of a second connecting part 11121.

A wiring part 1113 is laterally extended from a second connecting part 11121 in a direction away from the electrical functional component 11. The wiring part 1113 is located 5 between two columns of the electrode units 110, and a part of the wiring part 1113 is located in the open space 118 formed by the interval of the adjacent two columns of electrode units 110, so as to shorten the distance of the wiring part 1113 beyond the edge of the electrical functional component 11, resulting in a more compact arrangement of the electrical functional component 11, and avoiding the increase in manufacturing cost caused by increasing the overall size of the electrical functional component 11. The wiring part 1113 is arranged at intervals with the electrode units 110 adjacent thereto, which may provide more operation space for welding between the wiring part 1113 and the wire 14. The wiring part 1113 and the second connecting part 11121 are vertically arranged. The wiring part 1113 and the first connecting part 11120 are substantially arranged in parallel.

The first connecting parts 11120 are distributed between all two adjacent electrode units 110 arranged in columns, so as to realize the electrical connection between the electrode units 110 located in the same column. There is at least one second connecting part 11121 between the electrode units 110 in adjacent columns, so as to realize the electrical connection between the electrode units 110 arranged in each column. There are at least two second connecting parts 11121. All the second connecting parts 11121 located between two electrode units 110 arranged in rows may all be the second connecting parts 11121 that realize the electrical connection between the two adjacent electrode units 110, or may include second connecting parts 11121 that partially realize the electrical connection between two adjacent electrode units 110 and second connecting parts 11121 that only realize the connection but not the electrical connection between two electrode units 110.

In the present embodiment, the electrode units 110 are arranged in a matrix of three rows and three columns, and the number of the electrode units 110 is nine. Regardless of the dimension that the wiring part 1113 extends out of the array where the electrode units 110 are located, a minimum length of the electrical functional component 11 is 79 mm and a minimum width of the electrical functional component 11 is 65 mm. That is, regardless of the dimension that the wiring part 1113 extends out of the array where the electrode units 110 are located, all electrode units 110 of the electrical functional component 11 are distributed at intervals in an area of at least 79 mm×65 mm. Preferably, a first connecting part 11120 is located between two adjacent electrode units 110 arranged in columns, and a second connecting part 11121 is located between two adjacent electrode units 110 in the middle rows, so as to realize the electrical connection between nine electrode units 110. The electrode units 110 located at the two ends of each column are freely arranged and connected with only one first connecting part 11120. The electrical functional component 11 is substantially in a shape of the Chinese character “ [symbol] ”. The open space 118 is located in an area surrounded by four electrode units 110 in adjacent columns and adjacent rows. The electrical functional component 11 has four open spaces 118. The minimum area, available and permeable, of the open space 118 through which the wiring part 1113 passes is about 196 mm², and the minimum area of each of the other three open spaces 118 is about 314 mm².

In order to prevent the electrical functional component 11 from overlapping and affecting the therapeutic effect after the insulated electrode 100 being attached to the head, regardless of the dimension that the wiring part 1113 extends out of the array where the electrode units 110 are located, the electrical functional component 11 has a maximum length of 170 mm and a maximum width of 100 mm. The maximum dimension of the electrical functional component 11 is based on an average skull dimension of sampling statistics, which can be suitable for most patients. That is, regardless of the dimension that the wiring part 1113 extends out of the array where the electrode units 110 are located, all electrode units 110 of the electrical functional component 11 are distributed at intervals in an area of at most 170 mm×100 mm. The row spacing of the electrical units 110 of the electrical functional component 11 is at most 18.5 mm, and the column spacing of the electrical units 110 of the electrical functional component 11 is at most 53.5 mm. That is, the spacing between two adjacent electrode units 110 in the same column is at most 18.5 mm, and the spacing between two adjacent electrode units 110 in the same row is at most 53.5 mm. The maximum area, available and permeable, of the open space 118 through which the wiring part 1113 passes is about 2809 mm², and the maximum area of each of the other three open spaces 118 is about 3000 mm².

Preferably, the row spacing of the electrical units 110 of the electrical functional component 11 is 1.5 mm, and the column spacing of the electrical units 110 of the electrical functional component 11 is 24 mm. The diameter of the electrode unit 110 is 21 mm. Regardless of the dimension that the wiring part 1113 extends out of the array where the electrode units 110 are located, an area where the electrical functional component 11 is located has a length of 111 mm and a width of 66 mm. The wiring part 1113 has a width of 8 mm, and a distance from edges of the main body part 111 located on both sides of the wiring part 1113 is 8 mm, so as to provide sufficient wiring space and reduce the manufacturing difficulty. The widths of the first connecting part 11120 and the second connecting part 11121 are both 4.5 mm, which is sufficient for wiring design and making enough open spaces 118 between the electrode units 110 to facilitate heat and water vapor to escape. The length of the first connecting part 11120 is about 24.7 mm, and the length of the second connecting part 11121 is about 2.1 mm. The area of the open spaces 118 is about 903 mm². It can be understood that, an area, available and permeable, of the open spaces 118 through which the wiring part 1113 passes is about 652 mm².

When the insulated electrode 100 is used, the open spaces 118 may perform heat conduction and water evaporation to the skin surface, wherein the open spaces 118 are partially open for a complete permeability and heat dissipation, and are partially covered by a support member 13 and an adhesive 15 and thus heat is conducted through the support member 13 and the adhesive 15. During a process of the tumor treating fields therapy, the heat accumulated on the skin surface of a patient corresponding to the applied insulated electrode 100 and the water vapor generated by sweating can be discharged to the outside air through the open spaces 118, avoiding skin discomfort symptoms such as erythema, itching, hair follicle inflammation, pain and papules. In other embodiments, the implemented electrode unit 110 may also have other shapes, such as square or polygon. A range of the width of the electrode unit 110 in an extending direction of a row or a column is 21 mm to 22 mm, so that the electrode unit 110 can give consideration to both the effect of tumor treating fields and the adhesion between the insulated electrode 100 and the patient's skin.

In other embodiments, the second connecting parts 11121 include not only second connecting parts 11121 that realize the electrical connection between two adjacent electrode units 110 arranged in rows, but also second connecting parts 11121 that only serve to strengthen the connection rather than the electrically connection of the two adjacent electrode units 110 arranged in rows. The electrical functional component 11 is substantially in a shape of the Chinese character “ [symbol] ”.

In the present embodiment, the wiring part 1113 is welded with the wire 14, so as to realize the electrical connection between the electrical functional component 11 and the wire 14. Rows of golden fingers 11130 welded to the wire 14 are arranged in a staggered manner, separately, on two side surfaces of an end of the wiring part 1113 away from the second connecting part 11121. The periphery of the welding point of the wire 14 and the golden fingers 11130 of the wiring part 1113 is covered with a heat shrinkable sleeve 141. The heat shrinkable sleeve 141 provides support and insulation protection for the connecting point between the wire 14 and the wiring part 1113 of the electrical functional component 11, so as to prevent the connecting point between the wire 14 and the wiring part 1113 of the electrical functional component 11 from being broken. Meanwhile, it may also be dustproof and waterproof. The end of the wire 14 away from the second connecting part 11121 is configured with a plug 142 electrically connected to the electric field generator (not shown). One end of the wire 14 is electrically connected to the golden fingers 11130 of the wiring part 1113, and the other end of the wire 14 is electrically connected with the electric field generator (not shown) through the plug 142, so as to provide the insulated electrode 100 with alternating current signals for tumor treatment during the tumor treating fields therapy.

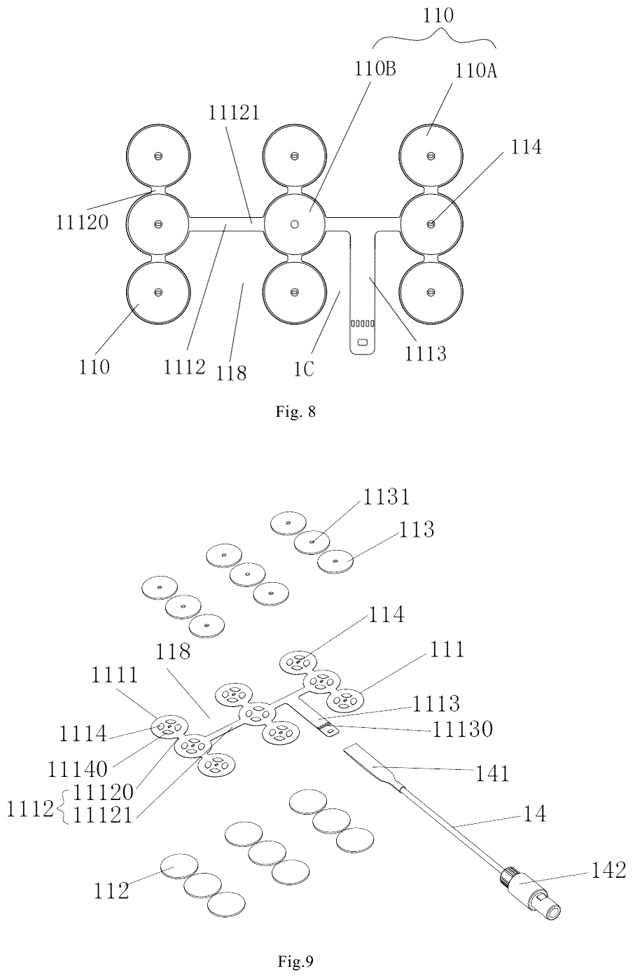

The electrode unit 110 includes: the main body part 1111 arranged at two opposite ends of the connecting part 1112; an insulating plate 112 arranged at one side of the main body part 1111 away from the human skin; a dielectric element 113 arranged at one side of the main body part 1111 facing the human skin; and a temperature sensor 114 selectively arranged on the main body part 1111 and located at the same side as the dielectric element 113. The main body part 1111, the insulating plate 112 and the dielectric element 113 are all in a circular sheet structure. The insulating plate 112, the main body part 1111 and the dielectric element 113 are arranged in one-to-one correspondence, and have centers located on the same straight line. The temperature sensor 114 may also be selectively disposed on the corresponding main body part 1111. That is, some electrode units 110 are provided with temperature sensors 114, and some electrode units 110 are not provided with temperature sensors 114. As shown in FIG. 8, the nine electrode units 110 can be divided into eight peripheral electrode units 110A and one central electrode unit 110B surrounded by the peripheral electrode units 110A. The eight peripheral electrode units 110A are provided with the temperature sensors 114, and the one central electrode unit 110B is not provided with the temperature sensor 114. In other embodiments, the main body part 1111 may also be a strip-shaped structure extending from an end of the connecting part 1112.

A side of the main body part 1111 facing the dielectric element 113 is provided with a conductive pad 1114. The conductive pad 1114 of the main body part 1111 may be completely covered by the dielectric element 113, so that the conductive pad 1114 and the dielectric element 113 can be welded by the solder 115. The conductive pad 1114 of the main body part 1111 includes a plurality of conductive cores 11140 center-symmetrically arranged, which may effectively prevent the offset of the position of the dielectric element 113 caused by the stacking of the solder 115 during the welding process. The center of the conductive pad 1114 of the main body part 1111 is located on the center line of the main body part 1111. The top surfaces of the plurality of conductive cores 11140 of the conductive pad 1114 are located on the same plane, which may avoid pseudo soldering when welding with the dielectric element 113. The center of the conductive pad 1114 is also located on the center line of the dielectric element 113.

In the present embodiment, a conductive pad 1114 of the same main body part 1111 includes four conductive cores 11140 center-symmetrically arranged at intervals. The conductive cores 11140 of the conductive pad 1114 are arranged in a multi-point interval mode, so that the consumption of cop foil for manufacturing the conductive cores 11140 can be reduced and the material cost is reduced. At the same time, the amount of solder 115 used for welding the conductive cores 11140 and the dielectric element 113 can be saved, further reducing the material cost.

The four conductive cores 11140 of the same conductive pad 1114 are all in petal-shaped structures. Each conductive core 11140 includes an inner arc (not labeled) and an outer arc (not labeled) connected end to end. The inner arc (not labeled) and the outer arc (not labeled) of the conductive core 11140 are axial-symmetrically arranged. Inner arcs (not labeled) of the four conductive cores 11140 of the same conductive pad 1114 are all recessed in a direction toward the center of the conductive pad 1114. Outer arcs (not labeled) of the four conductive cores 11140 of the same conductive pad 1114 all protrude in a direction away from the center of the conductive pad 1114. A plurality of conductive cores 11140 constituting the conductive pad 1114 are both center-symmetrically arranged and axial-symmetrically arranged, and each conductive core 11140 is also axial-symmetrically arranged, so that when the plurality of conductive cores 11140 of the conductive pad 1114 of the main body part 1111 are welded with the dielectric element 113, the stress balance of each welding point is guaranteed, ensuring an overall welding balance of the dielectric element 113 and improving the welding quality, so as to avoid an inclination of the dielectric element 113 caused by the unbalanced welding stress, which may result in a weak strength and easy fracture of the welding point on a side with larger spacing between the dielectric element 113 and the main body part 1111. At the same time, it may also avoid affecting the fit of the insulated electrode 100. Outer arcs (not labeled) of the plurality of conductive cores 11140 of the same conductive pad 1114 are substantially located on a same circumference.

The insulating plate 112 is made of an insulating material. Preferably, the insulating plate 112 is an epoxy glass cloth laminate plate. The insulating plate 112 is adhered to a surface of the main body part 1111 away from human skin by sealant (not shown), which can enhance the strength of the main body part 1111, provide a flat welding plane for the welding operation between the main body part 1111 and the dielectric element 113, and improve the product yield rate. At the same time, the insulating plate 112 may also isolate the water vapor in the air on the side of the insulated electrode 100 away from the skin from the solder 115 located between the main body part 1111 and the dielectric element 113, so as to prevent the water vapor from eroding the solder 115 between the main body part 1111 and the dielectric element 113, which may affect the electrical connection between the main body part 1111 and the dielectric element 113.

The size of the insulating plate 112 is the same as the size of the main body part 1111, so as to prevent the sealant (not shown) from climbing to the side of the main body part 1111 facing the human skin by capillary effect when the insulating plate 112 is adhered to a surface of the main body part 1111 away from human skin by sealant (not shown), which may affect the filling of the sealant 117 in a gap 116 formed by welding the dielectric element 113 and the main body part 1111, resulting the existence of cavities in the sealant 117. This may further prevent the sealant 117 from bursting due to the rapid expansion of water vapor caused by a large difference in thermal expansion coefficient between the sealant 117 and the water vapor in the cavities during high-temperature curing, which may lead to popcorn phenomenon, and damage the products.

The dielectric element 113 is made of a material with high dielectric constant which has conductive characteristics of blocking the conduction of direct current and allowing alternating current to pass through, which may ensure human safety. Preferably, the dielectric element 113 is a dielectric ceramic sheet with a dielectric constant of at least greater than 1000. The dielectric element 113 has an annular structure, with a perforation 1131 extending through the center thereof for accommodating the temperature sensor 114. An annular metal layer 1132 is attached to the surface of the dielectric element 113 facing the main body part 1111 (see FIG. 9). A point-to-side welding is formed between the metal layer 1132 of the dielectric element 113 and the conductive cores 11140 of the conductive pad 1114 of the main body part 1111, so that it is more convenient to weld without requiring higher welding alignment accuracy. The gap 116 formed by welding the dielectric element 113 and the main body part 1111 is filled with sealant 117 to protect the solder 115 between the dielectric element 113 and the main body part 1111, so as to prevent the dielectric element 113 from being affected by external force, which leads to the fracture of the welding part, and further leads to the failure of the alternating electric field to be applied to the patient's tumor site through the dielectric element 113. At the same time, it can also prevent the water vapor in the air from entering the gap 116 and eroding the solder 115 between the dielectric element 113 and the main body part 1111, which may affect the electrical connection between the dielectric element 113 and the main body part 1111. The inner ring of the metal layer 1132 of the dielectric element 113 and the edge of the perforation 1131 of the dielectric element 113 are arranged at intervals, so as to prevent the solder 115 between the metal layer 1132 of the dielectric element 113 and the main body part 1111 from spreading in a direction toward the perforation 1131 of the dielectric element 113 when heated and melted, which may result in a short circuit of the temperature sensor 114. The outer ring of the metal layer 1132 of the dielectric element 113 and the outer edge of the dielectric element 113 are also arranged at intervals, so as to prevent the solder 115 between the metal layer 1132 of the dielectric element 113 and the main body part 1111 from overflowing to the outside of the main body part 1111 when heated and melted, which may result in a direct current that is not hindered by the dielectric element 113 passes through and acts on the patient's body surface when the insulated electrode 100 is attached to the body surface of a patient's tumor site.

The outer diameter of the dielectric element 113 is slightly smaller than the diameter of the main body part 1111. When filling the sealant 117, the sealant 117 can be filled into the gap 116 along the edge of the main body part 1111 located outside the dielectric element 113 by capillary phenomenon, which is beneficial to the filling of the sealant 117 in the gap 116 formed by welding the dielectric element 113 and the main body part 1111. When filling the sealant 117 in the gap 116 formed by welding the dielectric element 113 and the main body part 1111, the air in the gap 116 may be exhausted from the perforation 1131 of the dielectric element 113, so as to prevent the sealant 117 filled in the gap 116 from generating cavities, and thus improve the product quality.

Referring to FIG. 8, there are a plurality of temperature sensors 114 respectively accommodated in perforations 1131 of the corresponding dielectric elements 113. In the present embodiment, the number of the temperature sensors 114 is eight. The electrode unit 110 of the present embodiment includes: one central electrode unit 110B located in the middle row and the middle column; and other eight peripheral electrode units 110A. It is easier for air to enter between the peripheral electrode units 110A and human skin, resulting in increasing the impedance and heat generation. Therefore, eight temperature sensors 114 are located on the eight peripheral electrode units 110A, respectively. Each temperature sensor 114 is located at the center of the main body part 1111 of the corresponding electrode unit 110. The temperature sensor 114 is used to monitor the temperature of the adhesive 15 covering the surface of the dielectric element 113 of the electrical functional component 11 facing the human skin, and further detect the temperature of the human skin to which the adhesive 15 is attached. When the temperature monitored by the temperature sensor 114 exceeds the upper limit of the safe temperature of human body, the tumor treating fields system 1000 may reduce or turn off the alternating current transmitted to the insulated electrode 100 in time, so as to avoid cryogenic burns of human body. The temperature sensors 114 are welded to the main body part 1111, and are then sealed with sealant 117, so as to prevent the temperature sensor 114 being eroding by water vapor and causing a failure of the temperature sensors 114. The temperature sensor 114 has a signal terminal (not shown) and a ground terminal (not shown). In the present embodiment, the temperature sensor 114 is preferably a thermistor. In other embodiments, the specific number of temperature sensors 114 can be set as required.

Referring to FIG. 8, the main body part 1111, the insulating plate 112 and the dielectric element 113 are all arranged in three rows and three columns. The main body part 1111 of the electrode unit 110 arranged in three rows and three columns, a plurality of connecting parts 1112 each located between two adjacent electrode units 110, and a wiring part 1113 extending outward from one connecting part 1112 together constitute the flexible circuit board 111 of the electrical functional component 11. In view of the formation of the electrode unit 110, the insulating plate 112 is arranged on the side of the main body part 1111 of the flexible circuit board 111 away from the human skin, and the dielectric element 113 is arranged on the side of the main body part 1111 of the flexible circuit board 111 facing the human skin. The temperature sensor 114 is selectively arranged on the side of the main body part 1111 of the flexible circuit board 111 facing the human skin. The insulating plate 112 and the dielectric element 113 are respectively arranged on opposite sides of the main body part 1111 of the flexible circuit board 111. The main body part 1111 of the flexible circuit board 111 of the electrical functional component 11 is arranged in accordance with the electrode units 110 of the electrical functional component 11.

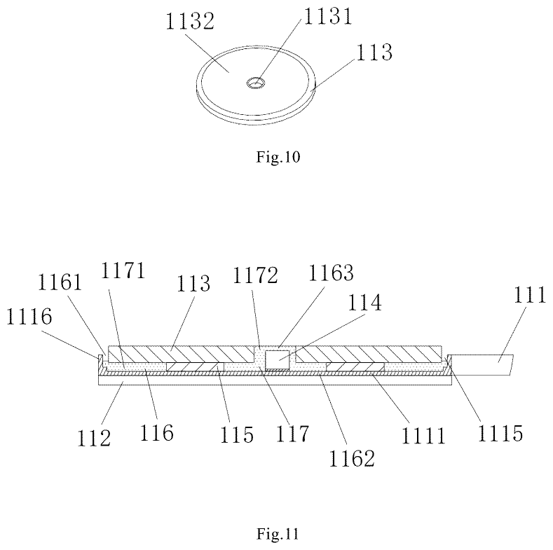

FIG. 10 is a sectional view of the electrode unit 110. The sealant 117 adopts a secondary sealing method. First, the sealant 117 is filled in the gap between the dielectric element 113 and the main body part 1111 from the edge of the dielectric element 113, in which the solders 115 are point coated, and the sealant 117 flows inward through the interspace between the solders 115 and covers the solders 115. A second sealing is performed after high-temperature curing. The second sealing is filling sealant at the perforation 1131 of the dielectric element 113, which may fully seal the temperature sensor 114.

FIG. 11 is a sectional structure of another electrode unit 110, which is basically the same as the electrode unit 110 in FIG. 10, and here the relevant reference numerals are used. The difference is that the main body part 1111 of the flexible circuit board 111 has a groove 1115. The groove 1115 is formed by a downward depression on a side of the main body part 1111 facing the dielectric element 113, and a sidewall 1116 is formed around the groove 1115. The dimension of the groove 1115 is larger than the dimension of the dielectric element 113, so that the dielectric element 113 is accommodated in the groove 1115 of the main body part 1111 of the flexible circuit board 111. A conductive pad (not labeled) is on the bottom surface in the groove 1115 and a solder 115 is located on the conductive pad. The conductive pad (not labeled) is welded and connected with the dielectric element 113 through the solder 115.

A gap 116 for accommodating the sealant 117 is formed between the dielectric element 113 and the main body part 1111 of the flexible circuit board 111, and the gap 116 is located in the groove 1115. The gap 116 includes: a first gap 1161 located between the dielectric element 113 and the sidewall 1116 of the main body part 1111 and being in a ring shape; a second gap 1162 formed between the bottom surface of the groove 1115 of the flexible circuit board 111 and the surface of the dielectric element 113 facing the flexible circuit board 111; and a third gap 1163 formed between the sidewall 1116 of the perforation 1131 of the dielectric element 113 and the temperature sensor 114 and being in a ring shape. The third gap 1163 is located above the second gap 1162. The first gap 1161 is communicated with the second gap 1162, and the second gap 1162 is communicated with the third gap 1163. The sealant 117 includes a first sealant 1171 and a second sealant 1172. The first sealant 1171 is filled in a direction from the first gap 1161 to the second gap 1162, and the second sealant 1172 is filled in a direction from the third gap 1163 to the second gap 1162. The first sealant 1171 and the second sealant 1172 together completely fill the second gap 1162, so as to ensure the reliability of the welding of the flexible circuit board 111 with the dielectric element 113 and the temperature sensor 114. When filling the second gap 1162 with the first sealant 1171, the first gap 1161 may accommodate the redundant first sealant 1171, so as to prevent the first sealant 1171 from overflowing and affecting the thickness of the insulating plate 112, which may lead to a false detection. And, it is not necessary to accurately control the amount of the first sealant 1171, thereby reducing the operation difficulty of filling the first sealant 1171. The first sealant 1171 is preferably an underfill. The second sealant 1172 is filled in the third gap 1163 and covers the temperature sensor 114, so as to prevent the water vapor from eroding the temperature sensor 114, which may cause the failure of the temperature sensor 114. A top plane of the second sealant 1172 is flush with the surface of the dielectric element 113 facing the patient's body surface, or is lower than the surface of the dielectric element 113 facing the patient's body surface.

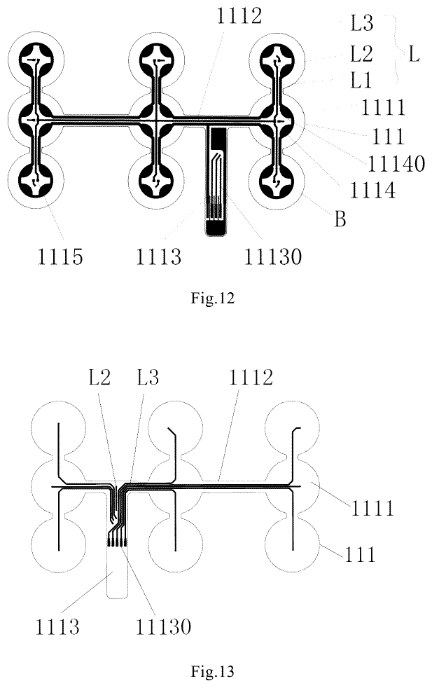

With reference to FIG. 12, the flexible circuit board 111 consists of an insulating substrate B; and a plurality of conductive traces L embedded in the insulating substrate B. The main body part 1111, the connecting part 1112 and the wiring part 1113 all consist of the corresponding insulating substrate B and a plurality of conductive traces L embedded in the insulating substrate B. The conductive trace L embedded in the insulating substrate B of the main body part 1111 is electrically connected with the conductive trace L embedded in the insulating substrate B of the connecting part 1112 and the conductive trace L embedded in the insulating substrate B of the wiring part 1113. The conductive core 11140 of the conductive pad 1114 arranged on the main body part 110 is exposed or protruded from the insulating substrate B of the main body part 110. The golden fingers 11130 of the wiring part 1113 are exposed from the insulating substrate B of the wiring part 1113. The insulating substrate B of the flexible circuit board 111 may isolate the water vapor in the air around the insulated electrode 100 from the solder 115 located between the conductive pad 1114 and the dielectric element 113, and prevent the water vapor in the air away from the skin from eroding the solder 115 located between the dielectric element 113 and the conductive pad 1114 on the main body part 1111 of the flexible circuit board 111. The insulating substrate B of the flexible circuit board 111 and the insulating plate 112 work as a double isolation, which may prolong the service life of the insulated electrode 100.

Mainly referring to FIG. 12 and FIG. 13, the conductive traces L of the flexible circuit board 111 are embedded in the insulating substrate B in a layered manner, including: a first conductive trace L1 that connects all conductive cores 11140 of the conductive pads 111 on the main body part 1111 in series, a second conductive trace L2 that connects all ground terminals (not shown) of the temperature sensors 114 located on the main body part 1111 in series, and a plurality of third conductive traces L3 that respectively connect signal terminals (not shown) of the temperature sensors 114 on the main body part 1111 in parallel. In the present embodiment, the first conductive trace L1 is provided with one path which connects all the conductive cores 11140 of the conductive pads 1114 located on respective main body parts 1111 in series, and is electrically connected with the corresponding golden fingers 11130 of the wiring part 1113 that expose the insulating substrate B. The second conductive trace L2 is provided with one path which connects the ground terminals (not shown) of the temperature sensors 114 located on respective main body parts 1111 in series. The third conductive traces L3 are provided with a plurality of paths which are respectively connected with the signal terminals (not shown) of respective temperature sensors 114 located on respective main body parts 1111, and connect the signal terminals (not shown) of respective temperature sensors 114 located on respective main body parts 1111 in parallel. Specifically, the third conductive traces L3 are provided with eight paths, the amount of which is the same as the number of the temperature sensors 114. The first conductive trace L1, the second conductive trace L2 and the third conductive traces L3 are electrically connected with the corresponding golden fingers 11130 of the wiring part 1113, respectively.

In view of wiring of the conductive traces L, the conductive traces L are arranged in the insulating substrate B of the flexible circuit board 111 in two layers, where the layer close to the patient's skin is defined as a first layer, and the layer away from the patient's skin is defined as a second layer. The part, located between the first layer and the second layer and connecting the corresponding part of the conductive trace on the first layer to the corresponding part of the conductive trace on the second layer, is defined as a conductive layer. The first conductive trace L1, which connects the conductive cores 11140 of all the conductive pads 1114 in series, is located on the first layer and is arranged at the periphery of the second conductive trace L2 by surrounding the second conductive trace L2. The connection part of the second conductive trace L2 which connects with the ground terminal (not shown) of the temperature sensor 114 is located on the first layer. The connection part of the second conductive trace L2 which connects with the corresponding golden fingers 11130 of the wiring part 1113 is also located on the first layer. The second conductive trace L2 first connects its connection part with the ground terminal (not shown) of the temperature sensor 114 to its corresponding part located on the second layer through a corresponding conductive layer, and then connects its corresponding part located on the second layer to a part of it located on the first layer and connected with the corresponding golden fingers 11130 of the wiring part 1113 through a corresponding other conductive layer, thereby by passing the first conductive trace L1 surrounding its corresponding part located on the first layer and avoiding crossing with the first conductive trace L1.

The third conductive traces L3 each connected to the signal terminal (not shown) of the corresponding temperature sensor 114 includes: a portion located on the second layer and electrically connected to the corresponding golden fingers 11130 of the wiring part 1113; a portion located on the first layer and connected to the signal terminal (not shown) of the corresponding temperature sensor 114; and a conductive layer connecting the portion located on the first layer and the portion located on the second layer. The part of the second conductive trace L2 located on the second layer is located between the corresponding parts of the plurality of third conductive traces L3 on the same layer. The corresponding part of the second conductive trace L2 located on the second layer is arranged close to the wiring part 1113, with three of the third conductive traces L3 wired on one side and five of the third conductive traces L3 wired on the other side.

Referring to FIG. 7, the support member 13 is adhered to the backing 12, and surrounds the outside of the dielectric element 113 of the electrode unit 110. In the center of the support member 13, through holes 130 are disposed therethrough for accommodating the dielectric elements 113 of the electrode units 110. Dielectric elements 113 of electrode units 110 located in the same column may be surrounded by the same support member 13. The support member 13 may be made of foam material to meet the permeability requirements of the insulated electrode 100. In the present embodiment, there are three support members 13, which are arranged at intervals and side by side, and are respectively arranged around the outside of the dielectric elements 113 of the electrode units 110 in different columns. The support members 13 are flush with the surface of a side of the electrode unit 110 away from the backing 12. That is, the support members 13 are flush with the surface of a side of the electrode unit 110 facing the adhesive 15. The spacing between the outer edge of the support member 13 and the through hole 130 is 1 mm to 3 mm. Considering the minimum column spacing and maximum column spacing of the electrical functional component 11, the minimum spacing between two adjacent support members 13 is 2 mm, and the maximum spacing is 51.5 mm.

The adhesive 15 has double-sided adhesiveness. One surface of the adhesive 15 is adhered to the surface of the support member 13 and the surface of the electrode unit 110 away from the backing 12. The other surface of that adhesive 15 is used as an adhesive lay, which is attached to the skin of human body surface to keep the skin surface moist and relieve local pressure. The adhesive 15 may preferably be a conductive adhesive to serve as a conductive medium. With the support of the support member 13, the adhesive 15 has better adhesion to human skin.

The adhesive 15 may contain anti-allergic medicinal substances, so as to alleviate the adverse reaction caused by the direct contact of the adhesive 15 with the skin. In the present embodiment, the anti-allergic medicinal substances may be corticosteroid medicinal substances, which is alkaline and has less side effects on the skin, and can alleviate the symptoms of skin erythema, itching, etc. Corticosteroid medicinal substances include Compound Ketoconazole Ointment, Mometasone Furoate Gel, Fluocinonide Ointment, Beclomethasone Cream, Compound Triamcinolone Acetonide Cream, and Fule Cream. In another embodiment, mupirocin can be used as the anti-allergic medicinal substance. Mupirocin is an antibiotic anti-infective medicinal substance, which can alleviate symptoms such as hair follicle inflammation, pain and papules. In the present embodiment, the adhesive 15 is made of conductive hydrogel. In the present embodiment, the adhesive 15 is composed of a copolymer of acrylamide, hydroxyl acrylate or hydroxyl methacrylate and crosslinking agent, deionized water, acetylene black or graphene powder, corticosteroids and mupirocin (pharmaceutical ingredients). The adhesive 15 containing medicinal substances is applied to the skin surface, and when it is in direct contact with the skin surface, the medicinal substances in the adhesive 15 will penetrate into the skin, thus relieving symptoms such as erythema, itching, hair follicle inflammation, pain and papules.

The insulated electrode 100 may further cover the outside of the adhesive 15 and the backing 12 with release papers 16 (as shown in FIG. 5), so as to protect the adhesive 15 and the backing 12 and avoid contamination of the adhesive 15 and the backing 12. The insulated electrode 100 may cover the adhesive 15 and the backing 12 with only one piece of release paper 16, or may cover the adhesive 15 and the backing 12 with two or more pieces of release papers 16 jointly. When using, the release papers 16 are teared off and the insulated electrode 100 is applied to the body surface corresponding to the human tumor site.

An alternate embodiment of the first embodiment of the insulated electrode

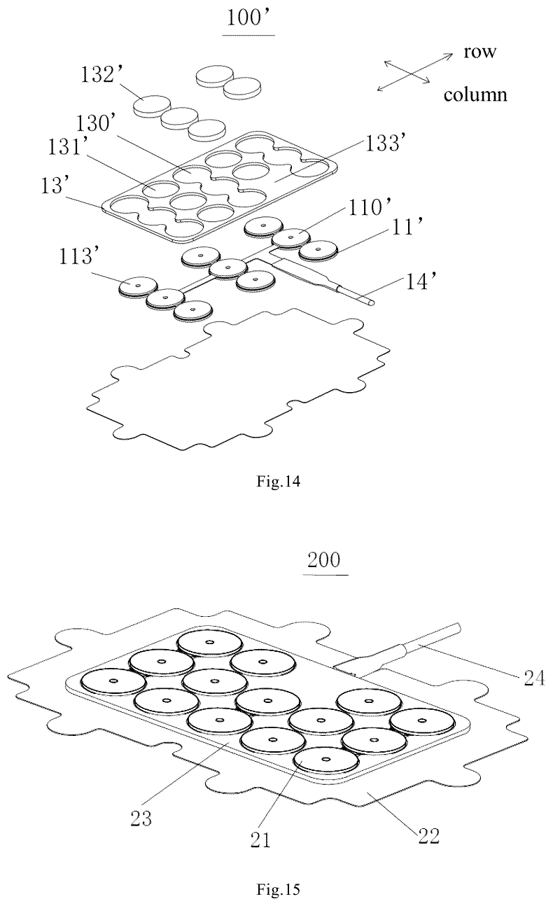

Referring to FIG. 14, the insulated electrode 100′ is an alternate embodiment of the insulated electrode 100 of the first embodiment. Similar to the insulated electrode 100 of the first embodiment, the insulated electrode 100′ includes, basically the same: a backing 12′; an electrical functional component 11′ arranged on the backing 12′; a wire 14′ electrically connected to the electrical functional component 11′; an adhesive (not shown) covering the electrical functional component 11′; and release papers (not shown) located above the adhesive (not shown) and attached to the backing 12′. The difference between the insulated electrode 100′ and the insulated electrode 100 of the first embodiment is that: the insulated electrode 100′ further include: at least one hygroscopic element 132′, arranged on the backing 12′ and located between a plurality of electrode units 110′ of the electrical functional component 11′ arranged at intervals. The hygroscopic element 132′ is used for absorbing and storing sweat or water vapor generated by a patient's body surface of corresponding site when the insulated electrode is applied, so as to avoid skin problems caused by the sweat or water vapor blocking hair follicle, and thus improve the comfort of applying the insulated electrode 100′. The support member 13′ of the insulated electrode 100′ is an integral sheet structure, and openings 131′ corresponding to the hygroscopic elements 132′ are formed on the support member 13′. The opening 131′ allows the corresponding hygroscopic element 132′ to pass through, and is used for accommodating the corresponding hygroscopic element 132′. The support member 13′ is provided with through holes 130′ which are the same as the through holes 130 of the support member 13 of the insulated electrode 100 in the first embodiment. Each opening 131′ is located between adjacent through holes 130′. The support member 13′ has a covering area 132′ covering the joint of the electrical functional component 11′ and the wire 14′. The openings 131′ for accommodating the hygroscopic elements 132′ are arranged away from the covering area 132′, so as to prevent the liquid absorbed by the hygroscopic elements 132′ from affecting the electrical connection between the wire 14′ and the electrical functional component 11′. The hygroscopic elements 132′ are located between a plurality of electrode units 110′ in adjacent columns. The thickness of the hygroscopic element 132′ may be slightly larger than the thickness of the support member 13′, so as to have better water absorption and storage performance.

The adhesive (not shown) attached to the support member 13′ may be an integral adhesive (not shown), the size of which is substantially the same as the support member 13′, and covers the support member 13′, the hygroscopic element 132′ and the dielectric element 113′ of the electrode unit 110′. As a simple alternative, the adhesive (not shown) may be three pieces of adhesive (not shown) respectively attached on the electrode units 110′ arranged in columns. Each piece of adhesive (not shown) is attached on the electrode units 110′ arranged in columns and on the site corresponding to the support member 13′.

The insulated electrodes 100, 100′ in the present embodiment transfer the alternating current signal to the dielectric elements 113, 113′ welded on the conductive pad 1114 through the conductive pad 1114 arranged on the flexible circuit board 11, and act on a patient's tumor site to realize the tumor treating fields therapy. The conductive pad 1114 has a plurality of conductive cores 11140 arranged symmetrically at intervals. By flattening the welding of the dielectric elements 113, 113′, it may avoid the inclination of the dielectric elements 113 to affect the fit of the insulated electrodes 100, 100′. At the same time, the consumption of cop foil for manufacturing the conductive pad 1114 can be reduced, and the amount of solder 115 used for welding the conductive pad 1114 and the dielectric elements 113, 113′ can be saved, reducing the manufacturing cost. Moreover, the insulated electrodes 100, 100′ of the present application have open spaces 118 located between a plurality of electrode units 110, 110′. During a process of the tumor treating fields therapy, the heat accumulated on the skin surface of a patient corresponding to the applied insulated electrodes 100, 100′ and the water vapor generated by sweating can be discharged to the outside air through the open spaces 118, avoiding skin discomfort symptoms such as erythema, itching, hair follicle inflammation, pain and papules.

### A Second Embodiment of the Insulated Electrode

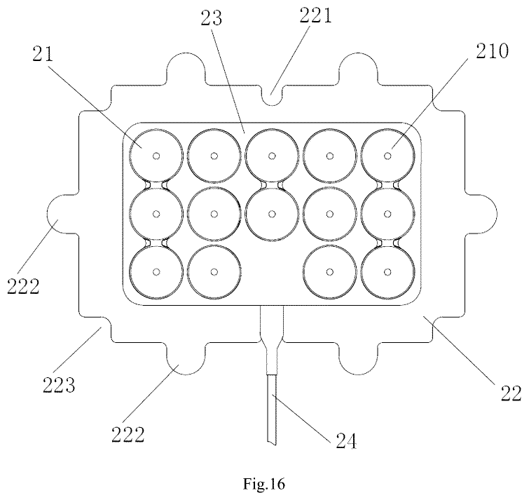

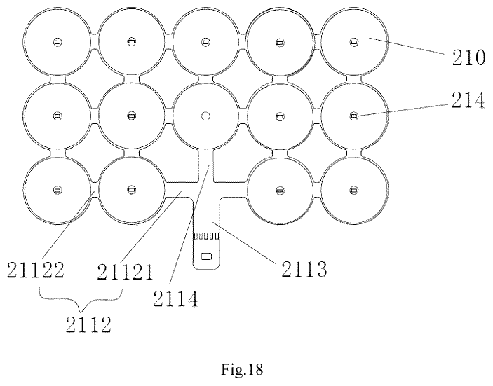

Referring to FIG. 15 to FIG. 19, the insulated electrode 200 of the present embodiment includes: a backing 22; an electrical functional component 21 adhered to the backing 22; a support member 23 adhered to the backing 22; an adhesive (not shown) adhered to the backing 22 and covering the corresponding parts of the support member 23 and the electrical functional component 21; and a wire 24 electrically connected to the electrical functional component 21. Herein, the backing 22 is exactly the same as the backing 12 of the insulated electrode 100 in the first embodiment. The edges of the backing 22 are provided with notches 221, lateral wings 222, concave corners 223, and other structures, which will not be repeated here. Reference may be made to the insulated electrode of the first embodiment for related contents.

The electrical functional component 21 is similar to the electrical functional component 11 of the insulated electrode 100 in the first embodiment, and includes: a plurality of electrode units 210 substantially arranged in a rectangular array; a plurality of connecting parts 2112 each located between adjacent electrode units 210 and electrically connected with two adjacent electrode units 210; and a wiring part 2113 extending from a connecting part 2112. Two adjacent electrode units 210 are connected to each other through the connecting part 2112, so that the electrical functional component 21 forms a mesh structure. A plurality of electrode units 210 are arranged in at least three rows and four columns. The number of electrode units 210 is at least ten. A plurality of connecting parts 2112 each connected with two adjacent electrode units 210 arranged in rows have different lengths or a plurality of connecting parts 2112 each connected with two adjacent electrode units 210 arranged in columns have different lengths. That is, two adjacent electrode units 210 arranged in rows have different spacings, or two adjacent electrode units 210 arranged in columns have different spacings. Specifically, a spacing between two adjacent electrode units 210 located in adjacent columns in the same row is different from a spacing between two adjacent electrode units 210 located in spaced columns in the same row. A spacing between two adjacent electrode units 210 located in adjacent rows in the same column is different from a spacing between two adjacent electrode units 210 located in spaced rows in the same column. Preferably, the spacing between two adjacent electrode units 210 located in adjacent columns in the same row is smaller than the spacing between two adjacent electrode units 210 located in spaced columns in the same row. The spacing between two adjacent electrode units 210 located in adjacent rows in the same column is smaller than the spacing between two adjacent electrode units 210 located in spaced rows in the same column. The spacing between two adjacent electrode units 210 located in adjacent columns in the same row is equal to the spacing between two adjacent electrode units 210 located in adjacent rows in the same column, ranging from 1 mm to 3 mm, preferably 2.1 mm. The connecting part 2112 includes: a first connecting part 21121 that connects two adjacent electrode units 210 and connects the wiring part 2113; and a plurality of second connecting parts 21122 that only connect two adjacent electrode units 210 in the same row or column. The wiring part 2113 is laterally extended from the first connecting part 21121 in a direction away from the electrode unit 210, and is electrically connected with the wire 24. The wiring part 2113 may be vertically arranged to the first connecting part 21121 or be vertically arranged to a corresponding part of the first connecting part 21121. A plurality of second connecting parts 21122 are substantially arranged in a shape of the Chinese character “-”, and may have the same length or may have different lengths. The second connecting parts 21122 connecting two adjacent electrode units 210 located in adjacent columns in the same row or connecting two adjacent electrode units 210 located in adjacent rows in the same column have the same length, and the length of a second connecting part 21122 is smaller than the length of the first connecting part 21121. The first connecting part 21121 may be in an “L” shape, located at the periphery of the electrical functional component 21, and connecting two electrode units 210 in adjacent columns or adjacent rows. Specifically, the first connecting part 21121 is in an “L” shape, which may connect two adjacent electrode units 210 located in adjacent rows and adjacent columns, or connect two electrode units 210 located in adjacent columns and spaced rows, or connect two electrode units 210 located in adjacent rows and spaced columns. The first connecting part 21121 may also be in a shape of the Chinese character “-”, connecting two adjacent electrode units 210 arranged in spaced columns and in the same row or connecting two adjacent electrode units 210 arranged in spaced rows and in the same column. The electrical functional component 21 may further include a reinforcing part 2114 with one end connected to the first connecting part 21121 and the other end connected to the electrode unit 210 corresponding to the first connecting part 21121. The reinforcing part 2114 and the first connecting part 21121 are in an “F” shape or a “T” shape. The reinforcing part 2114 and the wiring part 2113 are located on opposite sides of the first connecting part 21121, respectively. The reinforcing part 2114 may reinforce the strength of the wiring part 2113 arranged opposite thereto. The length of the reinforcing part 2114 is not less than the length of the second connecting part 21122. That is, the length of the reinforcing part 2114 is greater than or equal to the length of a second connecting part 21122 connecting two adjacent electrode units 210 in adjacent columns of the same row, or is greater than or equal to the length of a second connecting part 21122 connecting two adjacent electrode units 210 in adjacent rows of the same column.

In the present embodiment, the electrical functional component 21 includes: electrode units 210 arranged in three rows and five columns; and connecting parts 2112 connecting two adjacent electrode units 210 in the same row or the same column. There are fourteen electrode units 210 in total. In view of the row arrangement, the electrode units 210 include five electrode units 210 located in the first row, five electrode units 210 located in the middle row, and four electrode units 210 located in the last row. The connecting part 2112 located between two adjacent electrode units 210 in the first row or the middle row has the same length, and is in the range of 1 mm to 3 mm, preferably 2.1 mm. The connecting parts 2112 located between two adjacent electrode units 210 in the last row have different lengths, wherein the length of the connecting part 2112 located between two adjacent electrode units 210 in adjacent columns of the last row is equal to the length of the connecting part 2112 located between two adjacent electrode units 210 in the first row or the middle row, and the length of the connecting part 2112 located between two adjacent electrode units 210 in adjacent columns of the last row is smaller than the length of the connecting part 2112 located between two adjacent electrode units 210 in spaced columns of the last row. The length of the connecting part 2112 located between two adjacent electrode units 210 in adjacent columns of the last row is in the range of 1 mm to 3 mm, preferably 2.1 mm. The length of the connecting part 2112 located between two adjacent electrode units 210 in spaced columns of the last row is in the range of 22 mm to 27 mm.

In view of the column arrangement, the electrode units 210 have only two electrode units 210 in the middle column, and three electrode units 210 in each of the other four columns. The connecting parts 2112 connecting two adjacent electrode units 210 in each column have the same length, and are equal to the length of the connecting parts 2112 connecting two adjacent electrode units 210 in the first row or the middle row. The length of the connecting part 2112 connecting two adjacent electrode units 210 in each column is between 1 mm to 3 mm, preferably 2.1 mm. The lengths of the connecting parts 2112 between two adjacent electrode units 210 arranged in columns are all the same, ranging from 1 mm to 3 mm, preferably 2.1 mm. The lengths of the connecting parts 2112 located between two adjacent electrode units 210 arranged in rows are different. The length of the connecting part 2112 connecting two electrode units 210 located in adjacent columns of the same row is smaller than the length of the connecting part 2112 connecting two electrode units 210 arranged in spaced columns of the same row. The connecting parts 2112 between two adjacent electrode units 210 located in adjacent rows of the same column are all the second connecting parts 21122. The connecting parts 2112 between two adjacent electrode units 210 located in adjacent columns of the same row are also the second connecting part 21122. The length of the second connecting part is in the range of 1 mm to 3 mm, preferably 2.1 mm. The connecting parts 2112 between two adjacent electrode units 210 located in spaced columns of the same row are the first connecting parts 21121. Both the first connecting part 21121 and the second connecting part 21122 are in a shape of the Chinese character “-”. The length of the first connecting part 21121 is different from the length of the second connecting part 21122. The length of the first connecting part 21121 is larger than the length of the second connecting part 21122.

A wiring part 2113 is laterally extended from the first connecting part 21121 in a direction away from the electrical functional component 21. The wiring part 2113 and the first connecting part 21121 are vertically arranged. The wiring part 2113 and the first connecting part 21121 are in a “T” shape. The length of the first connecting part 21121 connecting two adjacent electrode units 210 in spaced columns of the same row is larger than the length of the second connecting part 21122 only connecting two adjacent electrode units 210 in adjacent columns of the same row. The first connecting part 21121 is electrically connected to the wiring part 2113. The electrical functional component 21 may further include a reinforcing part 2114 with one end connected to the first connecting part 21121 which is connected with the wiring part 2113 and the other end connected to the electrode unit 210 corresponding to the first connecting part 21121. Specifically, one end of the reinforcing part 2114 is connected with the electrode unit 210 located in the middle column of the middle row, and the other end of the reinforcing part 2114 is connected with the middle part of the first connecting part 21121. The reinforcing part 2114 and the first connecting part 21121 are in an inverted “T” shape. The reinforcing part 2114 and the wiring part 2113 are located on opposite sides of the first connecting part 21121, respectively, which may provide traction for the wiring part 2113 and prevent the application of the insulated electrode 200 from being affected by uneven force when the insulated electrode 200 is applied to the body surface corresponding to a patient's tumor site. The reinforcing part 2114 and the wiring part 2113 are located on the same straight line. The reinforcing part 2114 and the first connecting part 21121 are vertically arranged.

In the present embodiment, the electrode unit 210 has a substantially circular sheet structure. A diameter of the electrode unit 210 is about 21 mm. The length of the second connecting part 21122 is 1 mm to 3 mm. It may increase the number of the electrode units 210 in a unit area of the insulated electrode 200, and may increase the coverage area of the electrode units 210 of the insulated electrode 200 without increasing the overall area of the insulated electrode 200, so as to enhance the electric field intensity applied to the tumor site for TTF therapy, increase the coverage range of the alternating electric field to the tumor site, and improve the therapeutic effect. In the present embodiment, the lengths of the second connecting parts 21122 are all 2.1 mm. In another embodiment, the first connecting part 21121 is in a shape of the Chinese character “-”, which may be the connecting part 2112 connecting two adjacent electrode units 210 located in spaced rows of the same column or the connecting part 2112 connecting two adjacent electrode units 210 located in spaced columns of the same row. The second connecting part 21122 is the connecting part 2112 connecting two adjacent electrode units 210 located in adjacent columns of the same row or the connecting part 2112 connecting two adjacent electrode units 210 located in adjacent rows of the same column. In other embodiments, the first connecting part is substantially in an “L” shape, located at the corner of the electrical functional component 21 and connecting two electrode units 210 in adjacent columns. The second connecting part is in a shape of the Chinese character “-”, connecting two adjacent electrode units 210 located in adjacent columns of the same row or two adjacent electrode units 210 located in adjacent rows of the same column.

The wiring part 2113 is electrically connected to the wire 24. In the present implementation, rows of golden fingers 21130 welded to the wires 24 are arranged in a staggered manner, separately, on two side surfaces of an end of the wiring part 2113 away from the connecting part 2112. One end of the wire 24 is electrically connected to the golden fingers 21130 of the wiring part 2113, and the other end of the wire 24 is docked with the plug of the electric field generator (not shown), so as to provide the insulated electrode 200 with alternating current for tumor treatment during the TTF treatment. The corresponding part of the wiring part 2113 close to the connecting part 2112 is located between the two electrode units 210 in the middle of the last row, so as to shorten the distance of the wiring part 2113 beyond the edge of the electrode units 210 by using the space between the electrode units 210, thereby avoiding the increase in manufacturing cost caused by the oversized overall size of the electrical functional component 21. The wiring part 2113 and the electrode unit 210 adjacent to the wiring part 2113 are arranged at intervals, which may provide more operation space for welding the wiring part 2113 and the wire 24. The periphery of the welding point of the wire 24 and the golden fingers 21130 of the wiring part 2113 is covered with a heat shrinkable sleeve 241. The heat shrinkable sleeve 241 provides support and insulation protection for the connecting point between the wire 24 and the wiring part 2113 of the electrical functional component 21, so as to prevent the connecting point between the wire 24 and the wiring part 2113 of the electrical functional component 21 from being broken. Meanwhile, it may also be dustproof and waterproof.

The electrode unit 210 includes: a main body part 2111; an insulating plate 212 arranged at one side of the main body part 2111 away from human skin; a dielectric element 213 arranged at one side of the main body part 2111 facing human skin; and a temperature sensor 214 selectively arranged on the main body part 2111 and located at the same side as the dielectric element 213. A side of the main body part 2111 facing the dielectric element 213 is provided with a conductive pad 2115. The conductive pad 2115 includes a plurality of petal-shaped conductive cores 21150 center-symmetrically arranged, and the conductive cores 21150 are welded and connected with the dielectric element 213 through the solder. The temperature sensor 214 is welded onto the main body part 2111, and is located at the center of the conductive pad 2115. A perforation 2131 is configured in the middle of the dielectric element 213 for accommodating the temperature sensor 214. The electrode unit 210 is basically the same as the electrode unit 110 of the insulated electrode 100 in the first embodiment, and will not be repeated here. Reference may be made to the first embodiment for related contents.

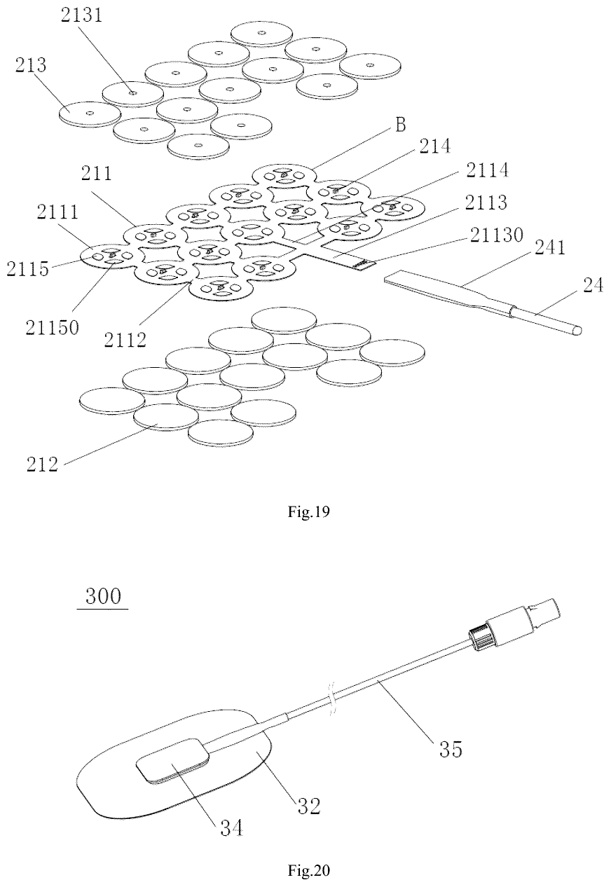

Referring to FIG. 19, there are a plurality of temperature sensors 214 respectively accommodated in the perforations 2131 of the corresponding dielectric elements 213. In the present embodiment, the number of the temperature sensors 214 is thirteen, which are respectively located on the thirteen electrode units 210 except the electrode unit 210 in the middle of the middle row. The thirteen temperature sensors 214 are respectively arranged at centers of the thirteen main body parts 2111.

The main body part 2111, the insulating plate 212 and the dielectric element 213 are all arranged in three rows and five columns. The main body part 2111 of the electrode unit 210 arranged in three rows and three columns, the plurality of connecting parts 2112 located between two adjacent electrode units 210, the wiring part 2113 extending outward from one connecting part 2112 and the reinforcing part 2114 corresponding to the wiring part 2113 together constitute the flexible circuit board 211 of the electrical functional component 21. In view of the formation of the electrode unit 210, the insulating plate 112 is arranged on the side of the main body part 2111 of the flexible circuit board 211 away from the human skin, and the dielectric element 213 is arranged on the side of the main body part 2111 of the flexible circuit board 211 facing the human skin. The temperature sensors 214 are selectively arranged on the side of the main body part 2111 of the flexible circuit board 211 facing the human skin. The main body parts 2111 of the flexible circuit board 211 of the electrical functional component 21 are arranged in consistent with the electrode units 110 of the electrical functional component 21.

The flexible circuit board 211 comprises: an insulating substrate B and a plurality of conductive traces (not shown) embedded in the insulating substrate B. Each the main body part 2111 and each wiring part 2113 both have an respective insulating substrate B and a plurality of respective conductive traces (not shown) embedded in the corresponding insulating substrate B. Each the connecting part 2112 and the reinforcing part 2114 both have an respective insulating substrate B. Each connecting part 2112 has a plurality of conductive traces (not shown) embedded in the corresponding insulating substrate B. The conductive traces (not shown) embedded in the insulating substrate B of the main body part 2111, the conductive traces (not shown) embedded in the insulating substrate B of the connecting part 2112, and the conductive traces (not shown) embedded in the insulating substrate B of the wiring part 2113 are electrically connected. Conductive traces (not shown) may be embedded in the insulating substrate B of the reinforcing part 2114. There may be no conductive trace (not shown) embedded in the insulating substrate B of the reinforcing part 2114, and the reinforcing part 2114 only reinforces the strength of the wiring part 2113. The plurality of connecting parts 2112 may also be in a form that: only some of the connecting parts 2112 have a plurality of conductive traces (not shown) embedded in the corresponding insulating substrates B, and some of the connecting parts 2112 have no conductive traces (not shown) embedded in the corresponding insulating substrates B.

The conductive traces (not shown) of the flexible circuit board 211 include: a path of the conductive trace (not shown) that connects all the conductive cores 21150 of the conductive pads 2115 located in each main body part 2111 in series, a path of the conductive trace (not shown) that connects all the ground terminals (not shown) of the temperature sensors 214 located on the main body parts 2111 in series, and a plurality of conductive traces (not shown) that connect all the signal terminals (not shown) of the temperature sensors 214 located on the main body parts 2111 in parallel. These conductive traces (not shown) are respectively and electrically connected with the corresponding golden fingers 21130 of the wiring part 2113. In order to facilitate the laying of conductive traces (not shown), the wiring part 2113 is wider than the connecting part 2112. Preferably, the width of the connecting part 2112 is 4 mm to 6 mm, and the width of the wiring part 2113 is 7 mm to 9 mm. In the present implementation, the width of the connecting part 2112 is 4.5 mm, and the width of the wiring part 2113 is 8 mm. It can be understood that some of the connecting parts 2112 may not be used for laying conductive traces (not shown), but only used for increasing the strength of the flexible circuit board 211.

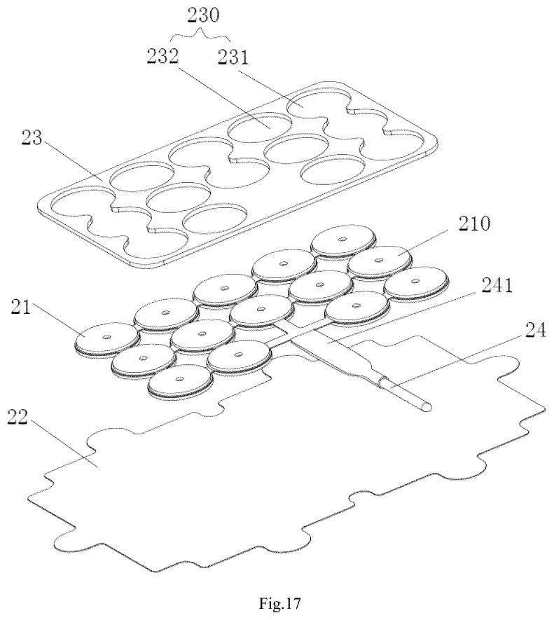

Referring to FIG. 17, the support member 23 is made of a one-piece foam. The support member 23 is provided with a plurality of through holes 230 corresponding to the electrode units 210 of the electrical functional component 21 for receiving the corresponding electrode units 210. The support member 23 surrounds each electrode unit 210 of the electrical functional component 21, which can improve the overall strength of the insulated electrode 200. The through holes 230 include a plurality of first through holes 231 and a plurality of second through holes 232. A plurality of first through holes 231 are arranged in a communicated manner and surround a plurality of electrode units 210 arranged in columns. The plurality of first through holes 231 may accommodate the connecting parts 2112 connecting two adjacent electrode units 210 in the same column, thus reducing the contact between the support member 23 and the connecting parts 2112 of the electrical functional component 21, and enabling the support member 23 to be attached to the backing 22 more smoothly. A plurality of second through holes 232 are arranged on the support member 23 at intervals and respectively surround the corresponding electrode unit 210 arranged in columns. In the present embodiment, the plurality of first through holes 231 respectively surround three electrode units 210 in the first column, two electrode units 210 in the third column and three electrode units 210 in the fifth column, respectively. The plurality of second through holes 232 surround respective electrode units 210 in the second column and the fourth column, respectively. The plurality of second through holes 232 are arranged in columns, and the second through holes 232 arranged in columns are arranged at intervals, so as to ensure the strength of the support member 23 and avoid being broken due to the external force. The first through hole 231 is substantially arranged in a racetrack shape.

The adhesive (not shown) is in one-piece, and has a size slightly larger than that of the support member 23. The adhesive (not shown) is preferably conductive gel. The adhesive (not shown) has double-sided adhesiveness, which may keep the skin surface moist and relieve local pressure when contacting with the skin.

In the present embodiment, the insulated electrode 200 applies an alternating electric field to a patient's tumor site by the 14 electrode units 210 arranged thereon for tumor treatment. It may avoid the influence of insufficient electric fields treating on the therapeutic effect caused by the difference of tumor size, site and position, increase the coverage area of the electrode units 210 of the insulated electrode 200, enhance the electric field intensity applied to the tumor site for TTF treatment, increase the coverage range of the alternating electric field to the tumor site, and improve the therapeutic effect.

### A Third Embodiment of the Insulated Electrode

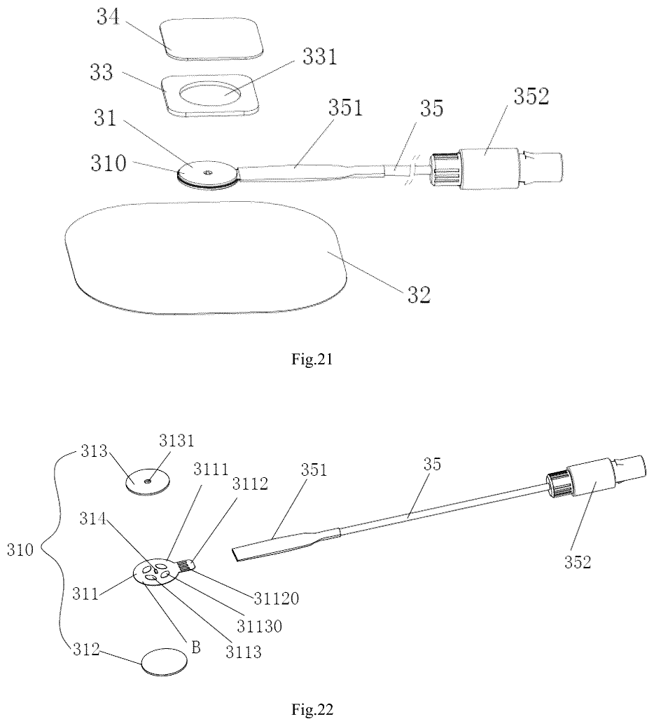

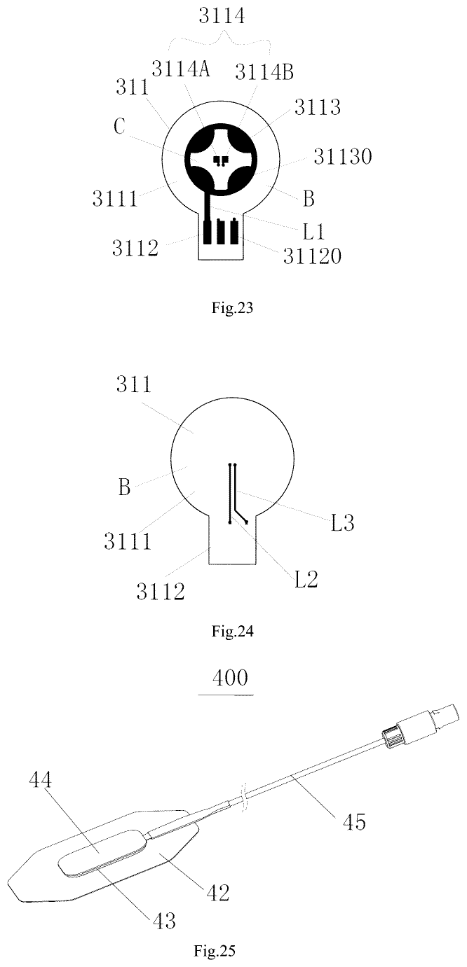

The electrical functional components of the insulated electrodes in the afore-mentioned first embodiment and the second embodiment are both equipped with a plurality of electrode units which are connected in series and if one of the electrode units is damaged, the entire insulated electrode will be scrapped, resulting in a high scrap cost. Therefore, the present embodiment further provides other forms of insulated electrodes. Referring to FIG. 20 to FIG. 24, the insulated electrode 300 of the present embodiment includes: a backing 32; an electrical functional component 31 adhered to the backing 32; a support member 33 adhered to the backing 32; an adhesive 34 that covers the site corresponding to the electrical functional component 31 and the support member 33 and adheres to the body's skin surface corresponding to the patient's tumor site; and a wire 35 electrically connected to the electrical functional component 31, in which the electrical functional component 31 is provided with only a single electrode unit 310 to reduce the manufacturing cost and scrap cost.

The electrical functional component 31 includes: a single electrode unit 310 in a circular sheet shape; and a wiring part 3112 connected with the electrode unit 310. The wiring part 3112 is welded with the wire 35 to realize the electrical connection between the electrical functional component 31 and the wire 35. A plurality of golden fingers 31120 are arranged on the surface of the wiring part 3112. In the present embodiment, the plurality of golden fingers 31120 are arranged on the surface of the wiring part 3112 facing the skin. The wire 35 is welded with the golden fingers 31120 of the wiring part 3112, and the periphery of the welding point is covered with a heat shrinkable sleeve 351. The end of the wire 35 away from the wiring part 3112 is configured with a plug 352 electrically connected to an electric field generator (not shown).

The backing 32 is substantially in a cubic sheet structure, and the four corners of the backing 32 are set in a rounded shape. The support member 33 is adhered to the backing 32, and surrounds the outside of the electrode unit 310. In the middle of the support member 33, a through hole 331 is disposed therethrough for accommodating the electrode unit 310. The adhesive 34 covers the surface of the support member 33 and the electrode unit 310 away from the backing 32, and is applied to the patient's skin.

The electrode unit 310 includes: a main body part 3111, an insulating plate 312, a dielectric element 313, and a temperature sensor 314. The main body part 3111 has a conductive pad 3113. The conductive pad 3113 includes four petal-shaped conductive cores 31130 center-symmetrically arranged at intervals. The dielectric element 313 has a perforation 3131 that accommodates the temperature sensor 314. The temperature sensor 314 is welded on the main body part 3111 and is accommodated in the perforation 3131 of the dielectric element 313. The specific structure of the electrode unit 310 is the same as that of the electrode unit 110 of insulated electrode 100 in the first embodiment, and will not be repeated here. Reference may be made to the first embodiment for related contents. The main body part 3111 included: an insulating substrate B, and three paths of conductive traces L embedded in the insulating substrate B. A layout of the three paths of the conductive traces L will be described in detail below. The three paths of conductive traces are a first conductive trace L1 arranged on a side of the insulating substrate B close to the dielectric element 313; and a second conductive trace L2 and a third conductive trace L3 both located on a side of the insulating substrate B close to the insulating plate 312. The diameter of the main body part 3111 is greater than 20 mm, preferably 21 mm. The plurality of conductive cores 31130 are all connected with the first conductive trace L1. The plurality of conductive cores 31130 are connected in series by the first conductive trace L1. Every two of the four conductive cores 31130 are arranged at intervals, and a spacing C is formed between two adjacent conductive cores 31130. The four spacings C are substantially in a cross shape. Adjacent spacings C are communicated. The extension direction of the two opposite spacings C is consistent with the extension direction of the wiring part 3112.

The main body part 3111 further includes a pair of pads 3114 that expose the insulating substrate B thereof. The pair of pads 3114 can be welded with the corresponding part of the temperature sensor 314 to realize the electrical connection between the temperature sensor 314 and the main body part 3111. The two pads 3114 are surrounded by four conductive cores 31130 of conductive pads 3113. The two pads 3114 are substantially located on the symmetrical center of the plurality of conductive cores 31130. One of the two pads 3114 is connected with the second conductive trace L2, and the other pad is connected with the third conductive trace L3. The first pad 3114A is the one of the two pads that connected with the second conductive trace L2, and the second pad 3114B is the pad that connected with the third conductive trace L3. The temperature sensor 314 has a signal terminal (not shown) and a ground terminal (not shown). The first pad 3114A is welded with the ground terminal (not shown) of the temperature sensor 314, and the second pad 3114B is welded with the signal terminal (not shown) of the temperature sensor 314.

The temperature sensor 314 is arranged on the main body part 3111 by welding the ground terminal (not shown) with the first pad 3114A of the main body part 3111, and welding the signal terminal (not shown) with the second pad 3114B of the main body part 3111. The first pad 3114A of the main body part 3111 is connected with the second conductive trace L2, the second pad 3114B is connected with the third conductive trace L3, the first pad 3114A is welded with the ground terminal (not shown) of the temperature sensor 314, and the second pad 3114B is welded with the signal terminal (not shown) of the temperature sensor 314, therefore, the ground terminal (not shown) of the temperature sensor 314 is electrically connected with the second conductive trace L2 of the main body part 3111, and the signal terminal (not shown) of the temperature sensor 314 is electrically connected with the third conductive trace L3 of the main body part 3111. That is, the temperature sensor 314 transmits signals through the second conductive trace L2 and the third conductive trace L3. The temperature sensor 314 is welded on the main body part 3111 and is accommodated in the perforation 3131 of the dielectric element 313.

The wiring part 3112 has the same structure as the main body part 3111, and also has a corresponding insulating substrate B and three paths of conductive traces L embedded in the insulating substrate B. The three paths of the conductive traces L of the wiring part 3112 are also respectively electrically connected to the corresponding conductive traces L of the main body part 3111. The wiring part 3112 has three golden fingers 31120, which expose the side of the insulating substrate B close to the dielectric element 313. The three paths of the conductive traces L of the wiring part 3112 are respectively electrically connected with the golden fingers 31120. The three paths of the conductive traces L of the wiring part 3112 are also the first conductive trace L1, the second conductive trace L2, and the third conductive traces L3, respectively. The first conductive trace L1 of the wiring part 3112 is extended from the first conductive trace L1 of the main body part 3111. The second conductive trace L2 of the wiring part 3112 is extended from the second conductive trace L2 of the main body part 3111. The conductive traces L3 of the wiring part 3112 is extended from the third conductive trace L3 of the main body part 3111.

The wiring part 3112 realizes the electrically connection with the conductive pad 3113 of the main body part 3111 by connecting the first conductive trace L1 thereof with the first conductive trace L1 of the main body part 3111, and connecting the first conductive trace L1 of the main body part 3111 with the conductive pad 3113 on the main body part 3111, and further realizes the electrically connection with the dielectric element 313 by welding the conductive pad 3113 of the main body part 3111 with the dielectric element 313. The wiring part 3112 realizes the electrically connection with the first pad 3114A on the main body part 3111 by connecting the second conductive trace L2 thereof with the second conductive trace L2 of the main body part 3111, and connecting the second conductive trace L2 of the main body part 3111 with the first pad 3114A on the main body part 3111, and further realizes the electrically connection with the ground terminal (not shown) of the temperature sensor 314 by welding of the first pad 3114A with the ground terminal (not shown) of the temperature sensor 314. The wiring part 3112 realizes the electrically connection with the second pad 3114B on the main body part 3111 by connecting the third conductive trace L3 thereof with the third conductive trace L3 of the main body part 3111, and connecting the third conductive trace L3 of the main body part 3111 with the second pad 3114B, and further realizes the electrically connection with the signal terminal (not shown) of the temperature sensor 314 by welding the second pad 3114B with the signal terminal (not shown) of the temperature sensor 314.

The main body part 3111 and the wiring part 3112 together constitute the flexible circuit board 311 of the electrical functional component 31. The main body part 3111 and the wiring part 3112 each has a corresponding insulating substrates B which together constitute the insulating substrate B of the flexible circuit board 311. The conductive traces L of the main body part 3111 and the conductive traces L of the wiring part 3112 are in one-to-one correspondence, and form the conductive trace L of the flexible circuit board 311. The insulating substrate B of the flexible circuit board 311 may isolate the water vapor in the air around the insulated electrode 300 from the solder (not shown) located between the conductive pad 3113 and the dielectric element 313, and prevent the water vapor in the air away from the skin from eroding the solder (not shown) located between the dielectric element 313 and the conductive pad 3113 on the main body part 3111 of the flexible circuit board 311. The insulating substrate B of the flexible circuit board 311 and the insulating plate 312 function as a double isolation, which may prolong the service life of the insulated electrode 300.

In view of the formation of the electrode unit 310, the insulating plate 312 is arranged on the side of the main body part 3111 of the flexible circuit board 311 away from the human skin, the dielectric element 313 is arranged on the side of the main body part 3111 of the flexible circuit board 311 facing the human skin, and the temperature sensor 314 is arranged on the side of the main body part 3111 of the flexible circuit board 311 facing the human skin. The insulating plate 312 and the dielectric element 313 are respectively arranged on opposite sides of the main body part 3111 of the flexible circuit board 311. The first conductive trace L1 of the flexible circuit board 311 connects the four spaced conductive cores 31130 of the conductive pad 3113 in series. The second conductive trace L2 is electrically connected to the ground terminal (not shown) of the temperature sensor 314 through the first pad 3114A. The third conductive trace L3 is electrically connected to the signal terminal (not shown) of the temperature sensor 314 through the second pad 3114B. The first conductive trace L1 is located in the insulating substrate B at a layer close to the human skin. The second conductive trace L2 and the third conductive trace L3 are located in the insulating substrate B at a layer close to the insulating plate 312. In order to facilitate the laying of the conductive traces L, the width of the wiring part 3112 is 7 mm to 9 mm. Preferably, the width of the wiring part 3112 is 8 mm.

The golden fingers 31120 of the wiring part 3112, the plurality of conductive cores 31130 of the conductive pad 3113 and the pad 3114 all expose the side of the insulating substrate B of the flexible circuit board 311 close to the dielectric element 313. The golden fingers 31120, the plurality of conductive cores 31130 of the conductive pad 3113 and the pad 3114 are all located on the side of the flexible circuit board 311 close to the patient's body surface. One golden finger 31120 of the wiring part 3112 has one end electrically connected with the dielectric element 313 through the first conductive trace L1 connected thereto and the other end welded with the corresponding part of the wire 35, so as to transmit the alternating electrical signal generated by the electric field generator (not shown) to the dielectric element 313. One of the other two golden fingers 31120 of the wiring part 3112 has one end electrically connected to the ground terminal (not shown) of the temperature sensor 314 through the second conductive trace L2 connected thereto, and the other one of the other two golden fingers 31120 has one end electrically connected to the signal terminal (not shown) of the temperature sensor 314 through the third conductive trace L3 connected thereto. The other two golden fingers 31120 of the wiring part 3112 each has the other end respectively welded with the corresponding parts of the wire 35. Thus, it realizes that the related signals detected by the temperature sensor 314 are transmitted to the electric field generator (not shown) through the second conductive trace L2, the third conductive trace L3 and the wire 35.

In the present embodiment, since the insulated electrode 300 uses a separate electrode unit 310 to apply an alternating electrical signal to the patient's tumor site, when it doesn't work properly, it only needs to replace the insulated electrode 300 with the separate electrode unit 310 without needing to scrap the entire insulated electrode containing a plurality of electrode units 310, which can reduce the cost of tumor treatment for a patient. In addition, the insulated electrode 300 of the present embodiment may be freely combined in quantity according to the patient's tumor site and the size of the patient's tumor site, ensuring the coverage area of the insulated electrode 300 for tumor treating fields therapy and ensuring the required electric field strength of the tumor treating fields therapy. Moreover, the relative positions of the plurality of insulated electrodes 300 may also be freely adjusted according to the patient's own physical differences, tumor site, and tumor size, in order to obtain the optimal electric field strength and coverage area for tumor treatment. At the same time, it may allow the skin on the patient's body surface to which the insulated electrode 300 is attached to breathe freely, avoiding the accumulation of heat on the patient's body surface caused by a long-term tumor treating fields therapy, and not being able to dissipate in time, which causes sweating and clogging pores and results in skin inflammation.

Furthermore, the flexible circuit board 311 of the insulated electrodes 300 is only provided with: a path of the first conductive trace L1 electrically connected with the dielectric element 313; a path of the second conductive trace L2 electrically connected with the ground terminals (not shown) of the temperature sensors 314; and a path of the third conductive traces L3 electrically connected with the signal terminals (not shown) of the temperature sensors 314. It realizes to transmit the alternating electrical signal generated by the electric field generator (not shown) to the dielectric element 313 through the first conductive trace L1, and realize the purpose of applying the alternating electrical signal to the patient's tumor site for tumor treatment. At the same time, it realizes the signal transmission between the electric field generator (not shown) and the temperature sensors 314 by electrically connecting the second conductive trace L2 and the third conductive trace L3 with the temperature sensors 314, respectively. Thus, the difficulty of wiring design is low, the structure is simple, the manufacturing process is simplified, the manufacturing is easy, and the product manufacturing yield is high, which may greatly reduce the manufacturing cost.

### A Fourth Embodiment of the Insulated Electrode

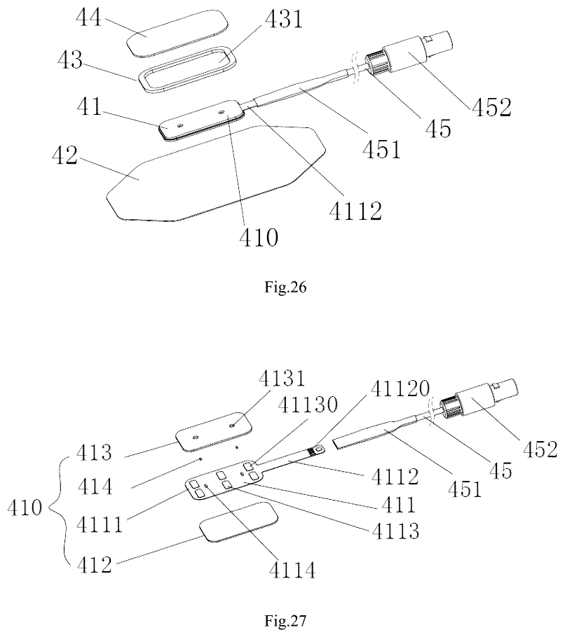

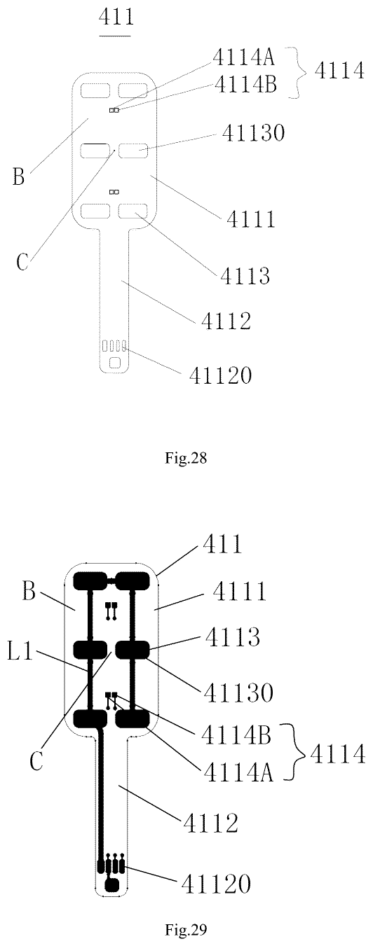

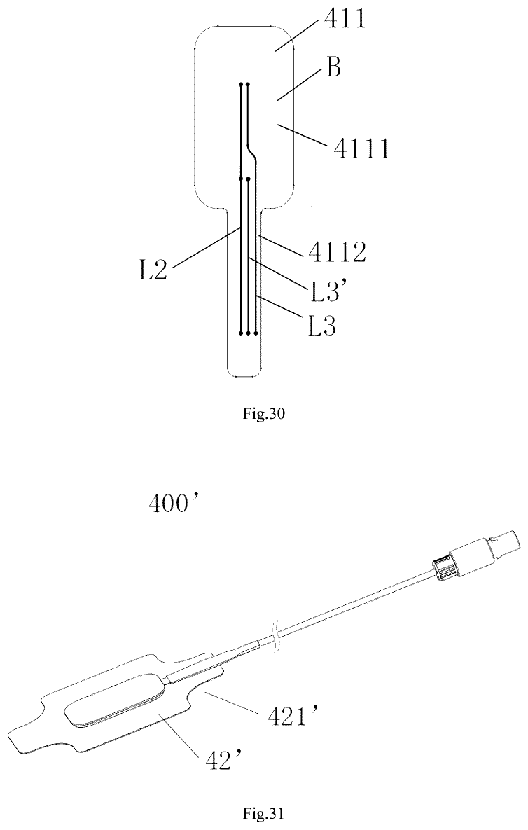

Referring to FIG. 25 to FIG. 30, the present embodiment is another insulated electrode 400 with only a single electrode unit, which includes: a backing 42; an electrical functional component 41 adhered to the backing 42; a support member 43 adhered to the backing 42; an adhesive 44 that covers the site corresponding to the electrical functional component 41 and the support member 43 and adheres to the body surface skin corresponding to the patient's tumor site; and a wire 45 electrically connected to the electrical functional component 41. Among them, the backing 42, the support member 43 and the adhesive 44, except for slightly differences in appearance, have the same functions and materials as the backing 12, the support member 13, and the adhesive 15 of the insulated electrode 100 in the first embodiment, respectively, and will not be repeated here. Reference may be made to the first embodiment for related contents.

The electrical function component 41 includes a single electrode unit 410 with a rectangular sheet shape and a wiring part 4112 connected with the electrode unit 410. The support member 43 has a through hole 431 disposed therethrough for accommodating the electrode unit 410. The wiring part 4112 is welded with the wire 45 to realize the electrical connection between the electrical functional component 41 and the wire 45. Four golden fingers 41120 are arranged on the surface of the wiring part 4112 facing the skin. The periphery of the welding point of the wire 45 and the golden fingers 41120 of the wiring part 4112 is covered with a heat shrinkable sleeve 451. The end of the wire 45 away from the wiring part 4112 is configured with a plug 452 electrically connected to an electric field generator (not shown) or a hub (not shown).

The electrode unit 410 includes a main body part 4111 arranged at the end of the wiring part 4112; an insulating plate 412 arranged at one side of the main body part 4111 away from the human skin; a dielectric element 413 arranged at one side of the main body part 4111 facing the human skin; and two temperature sensors 414 arranged on the main body part 4111 and located at the same side as the dielectric element 413. The main body part 4111 and the wire 45 are respectively arranged at opposite ends of the wiring part 4112. The dielectric element 413 is provided with two perforations 4131 therethrough for accommodating the corresponding temperature sensors 414 respectively, in which the number of the perforations 4131 is the same as the number of the temperature sensors 414. The main body part 4111, the insulating plate 412 and the dielectric element 413 have almost the same shapes and are all in the structure of a rectangular sheet. The main body part 4111, the insulating plate 412, and the dielectric element 413 are correspondingly arranged along the thickness direction of the main body part 4111, and have corresponding centers located on the same straight line. In the present embodiment, the main body part 4111, the insulating plate 412 and the dielectric element 413 are all in the structure of a rectangular sheet with rounded corners. Preferably, the main body part 4111 is in the structure of a rectangular sheet with a dimension of about 43.5 mm×23.5 mm. The wiring part 4112 of the electrical function component 41 is laterally extended from the main body part 4111 of the electrode unit 410. In other implementations, the main body part 4111 may also be in a strip-shaped or band-shaped structure extended from the end of the wiring part 4112.

The main body part 4111 is centrally provided with a conductive pad 4113. A metal layer (not shown) is attached to the surface of the dielectric element 4113 facing the main body part 4111. The conductive pad 4113 is welded with the dielectric element 413 to assemble the dielectric element 413 on the main body part 4111. The conductive pad 4113 may be completely covered by the dielectric element 413, so that the conductive pad 4113 and the dielectric element 413 can be welded by solder (not shown). The center of the conductive pad 4113 is located on the center line of the main body part 4111. The conductive pad 4113 includes a plurality of conductive cores 41130 center-symmetrically arranged, which may effectively prevent the offset of the position of the dielectric element 413 caused by the stacking of the solder (not shown) during the welding process. The top surfaces of the plurality of conductive cores 41130 are located on the same plane, which may avoid pseudo soldering when welding with the dielectric element 413. The plurality of conductive cores 41130 are all connected with a first conductive trace L1. The plurality of conductive cores 41130 are connected in series by the first conductive trace L1.

The conductive pad 4113 of the main body part 4111 are substantially in a rectangle structure, and has symmetry axes respectively coincident with the corresponding symmetry axis of the main body part 4111. The conductive pad 4113 includes six conductive cores 41130 located at four corners thereof and in the middle of two long sides thereof and arranged at intervals. The conductive core 41130 are arranged in a multi-point interval mode, so that the consumption of cop foil for manufacturing the conductive cores 41130 can be reduced. At the same time, the amount of solder (not shown) used for welding the conductive cores 41140 and the dielectric element 413 can be saved, reducing the manufacturing cost. Each conductive core 41130 is a rectangular structure with a dimension of approximately 8 mm×4 mm. Preferably, each conductive core 41130 is constructed in a rectangular shape with rounded corners. The longitudinal axis of each conductive core 41130 is parallel to the extension direction of the wiring part 4112. In other embodiments, the shape of each conductive core 41130 of the conductive pad 4113 may also be circular, square, etc.

In the present embodiment, the six conductive cores 41130 constituting the conductive pad 4113 are arranged in a matrix at intervals, and the six conductive cores 41130 are arranged in three rows and two columns along the longitudinal direction of the main body part 4111. There are two conductive cores 41130 in the first row, two conductive cores 41130 in the middle row, and two conductive cores 41130 in the last row. The spacing between two columns of conductive cores 41130 is about 2.4 mm, and the spacings between conductive cores 41130 in adjacent rows are all about 12.8 mm. The six conductive cores 41130 constituting the conductive pad 4113 are both center-symmetrically arranged and axial-symmetrically arranged, and each conductive core 41130 is also axial-symmetrically arranged, so that when the six conductive cores 41130 of the main body part 4111 are welded with the dielectric element 413, the stress at each welding point is balanced, ensuring an overall welding balance of the dielectric element 413 and improving the welding quality. Therefore, it may avoid an inclination of the dielectric element 413 caused by the unbalanced welding stress, which may result in a weak strength and easy fracture of the welding point on a side with a larger spacing between the dielectric element 413 and the main body part 4111. At the same time, it may also avoid affecting the fit of the insulated electrode 400. The six conductive cores 41130 of the conductive pad 4113 are arranged at intervals, and a spacing C is formed between two adjacent conductive cores 41130. Every two of the four conductive cores 41130 located in adjacent rows are arranged at intervals, and the four spacings C located between the four conductive cores 41130 are communicated and arranged in a cross shape. The dimension of the spacing C between two adjacent conductive cores 41130 in the same column is larger than the dimension of the spacing C between two conductive cores 41130 in the same row. Seven spacings C are formed between the six conductive cores 41130, and the seven spacings C are connected and arranged substantially in a “≠” shape. Adjacent spacings C are also arranged in a communicated manner. Among the seven spacings C, three of the spacings C each located between two adjacent conductive cores 41130 in the same row are at a straight line in a direction consistent with the extension direction of the wiring part 4112.

The main body part 4111 is provided with two pairs of pads 4114, which are respectively welded with corresponding parts of two temperature sensors 414, so as to realize the electrical connection between the temperature sensors 414 and the main body part 4111. Each pair of pads 4114 is located at the corresponding communication area of four spacings C formed by four conductive cores 41130 located in adjacent rows. The line connecting respective symmetrical centers of the two pairs of pads 4114 is at a straight line in a direction consistent with the extension direction of the wiring part 4112. The line connecting the two symmetrical centers of the two pairs of pads 4114 is at a straight line coincident with the longitudinal axis of the main body part 4111. The line connecting the two symmetrical centers of the two pairs of pads 4114 is at a straight line coincident with the longitudinal axis of the conductive pad 4113. The four conductive cores 41130 in the first row and the middle row are center-symmetrically arranged, and the four conductive cores 41130 in the middle row and the last row are also center-symmetrically arranged. The two pairs of pads 4114 are arranged offset from the symmetrical center of the four conductive cores 41130 located in two adjacent rows. Specifically, one pair of pads 4114 is arranged on a side of the symmetrical center of the rectangle away from the wiring part 4112, where the rectangle is formed by the four conductive cores 41130 in the first row and the middle row. The other pair of pads 4114 is arranged on a side of the symmetrical center of the rectangle close to the wiring part 4112, where the rectangle is formed by the four conductive cores 41130 in the middle row and the last row. Each pair of pads 4114 includes a first pad 4114A and a second pad 4114B. The temperature sensor 414 has a signal terminal (not shown) and a ground terminal (not shown). The ground terminal (not shown) of the temperature sensor 414 is welded with the first pad 4114A, and the signal terminal (not shown) is welded with the second pad 4114B, so that the temperature sensor 414 is electrically connected with the main body part 4111.

One of the two temperature sensors 414 is located at the communication area of four spacings C between the four conductive cores 41130 in the first row and the middle row, and the other is located at the communication area of four spacings C between the four conductive cores 41130 in the middle row and the last row. One temperature sensor 414 located in the surrounding area of the four conductive cores 41130 in the first row and the middle row is located at a side of the symmetrical center of the surrounding area of the four conductive cores 41130 in the first row and the middle row away from the wiring part 4112. The other temperature sensor 414 located in the surrounding area of the four conductive cores 41130 in the middle row and the last row is located at a side of the symmetrical center of the surrounding area of the four conductive cores 41130 in the middle row and the last row close to the wiring part 4112. Both of the two temperature sensors 414 are located in an area surrounded by the conductive pad 4113.

The main body part 4111 includes an insulating substrate B and four paths of conductive traces L embedded in the insulating substrate B. The four paths of conductive traces include: one path of a first conductive trace L1 arranged on a side of the insulating substrate B close to the dielectric element 413, one path of a second conductive trace L2 arranged on a side of the insulating substrate B close to the insulating plate 412, and two paths of third conductive traces L3 and L3′ arranged on the same side as the second conductive trace L2. The conductive pad 4113 of the main body part 4111 exposes the insulating substrate B, and the first conductive trace L1 connects the six conductive cores 41130 of the conductive pad 4113 in series. The two pairs of pads 4114 also expose the insulating substrate B. The two first pads 4114A are electrically connected with the second conductive trace L2, and the two second pads 4114B are respectively electrically connected with the two third conductive traces L3 and L3′. Therefore, the ground terminals (not shown) of the two temperature sensors 414 are all electrically connected with the second conductive trace L2 of the main body part 4111, and the signal terminals (not shown) of the two temperature sensors 414 are respectively electrically connected with the third conductive trace L3 and L3′ of the main body part 4111. The two temperature sensors 414 transmit the monitored temperature signals in parallel through the second conductive trace L2 and the third conductive traces L3 and L3′. The two temperature sensors 414 are welded on the main body part 4111 and are respectively accommodated in the corresponding perforations 4131 of the dielectric element 413. Preferably, the temperature sensor 414 is a thermistor.

The wiring part 4112 has the same structure as the main body part 4111, and also has a corresponding insulating substrate B and four paths of the conductive traces L embedded in the insulating substrate B. The four paths of the conductive traces L of the wiring part 4112 are respectively electrically connected to the corresponding conductive traces L of the main body part 4111 in one-to-one correspondence. The four golden fingers 41120 of the wiring part 4112 all expose the side of the insulating substrate B close to the dielectric element 413. The four paths of the conductive traces L of the wiring part 4112 are respectively electrically connected with the golden fingers 41120. The four paths of the conductive traces L of the wiring part 4112 are also the first conductive trace L1, the second conductive trace L2, and the third conductive traces L3 and L3′, respectively. The first conductive trace L1 of the wiring part 4112 is extended from the first conductive trace L1 of the main body part 4111. The second conductive trace L2 of the wiring part 4112 is extended from the second conductive trace L2 of the main body part 4111. The third conductive traces L3, L3′ of the wiring part 4112 are respectively extended from the corresponding third conductive traces L3, L3′ of the main body part 4111.

The wiring part 4112 realizes the electrically connection with the conductive pad 4113 of the main body part 4111 by connecting the first conductive trace L1 thereof with the first conductive trace L1 of the main body part 4111, and connecting the first conductive trace L1 of the main body part 4111 with the conductive pad 4113 on the main body part 4111, and further realizes the electrically connection with the dielectric element 413 through the welding of the conductive pad 4113 of the main body part 4111 and the dielectric element 413. The wiring part 4112 realizes the electrically connection with the first pad 4114A on the main body part 4111 by connecting the second conductive trace L2 thereof with the second conductive trace L2 of the main body part 4111, and connecting the second conductive trace L2 of the main body part 4111 with the first pad 4114A on the main body part 4111, and further realizes the electrically connection with the ground terminal (not shown) of the temperature sensor 414 through the welding of the first pad 4114A and the temperature sensor 414. The wiring part 4112 realizes the electrically connection with the two second pads 4114B on the main body part 4111 by respectively connecting the third conductive traces L3, L3′ thereof with the corresponding third conductive traces L3, L3′ of the main body part 4111, and respectively connecting the third conductive traces L3, L3′ of the main body part 4111 with the corresponding second pads 4114B, and further realizes the parallel electrically connection with the signal terminals (not shown) of the two temperature sensors 414 by respectively welding the two second pads 4114B with the corresponding signal terminals (not shown) of the two temperature sensors 414. Thus, the temperature signals monitored by the two temperature sensors 414 can be rapidly transmitted to the electric field generator (not shown) in parallel, so that the electric field generator (not shown) may timely and efficiently adjust the alternating electrical signal or alternating current applied to the dielectric element 413 to avoid cryogenic burns caused by excessively high temperature.

The main body part 4111 and the wiring part 4112 together constitute the flexible circuit board 411 of the electrical functional component 41. The respective insulating substrates B of the wiring part 4112 and the main body part 4111 together constitute the insulating substrate B of the flexible circuit board 411. The conductive trace L of the main body part 4111 and the conductive trace L of the wiring part 4112 are arranged in one-to-one correspondence and constitute the conductive trace L of the flexible circuit board 411.

In view of the formation of the electrode unit 410, the insulating plate 412 is arranged on the side of the main body part 4111 of the flexible circuit board 411 away from the human skin, the dielectric element 413 is arranged on the side of the main body part 4111 of the flexible circuit board 411 facing the human skin, and the two temperature sensors 414 are arranged on the side of the main body part 4111 of the flexible circuit board 411 facing the human skin. The insulating plate 412 and the dielectric element 413 are respectively arranged on opposite sides of the main body part 4111 of the flexible circuit board 411. The first conductive trace L1 of the flexible circuit board 411 connects the six spaced conductive cores 41130 of the conductive pad 4113 in series. The second conductive trace L2 is electrically connected to the ground terminals (not shown) of the two temperature sensors 314, respectively, through two first pads 4114A. The third conductive traces L3, L3′ are electrically connected to the signal terminals (not shown) of the two temperature sensors 314, respectively, through two second pads 4114B. The first conductive trace L1 is located in the insulating substrate B at a layer close to the human 25 skin. The second conductive trace L2 and the third conductive traces L3, L3′ are located in the insulating substrate B at a layer close to the insulating plate 412. In order to facilitate the laying of the conductive traces L, the width of the wiring part 4112 is 7 mm to 9 mm. Preferably, the width of the wiring part 4112 is 8 mm.

The golden fingers 41120 of the wiring part 4112, the six conductive cores 41130 of the conductive pad 4113, and the pads 4114 all expose the side of the insulating substrate B of the flexible circuit board 411 close to the dielectric element 413. The golden fingers 41120, the six conductive cores 41130 of the conductive pad 4113, and the pads 4114 are all located on the side of the flexible circuit board 411 close to the patient's body surface. One golden finger 41120 of the wiring part 4112 has one end electrically connected with the dielectric element 413 through the first conductive trace L1 connected thereto and the other end welded with the corresponding part of the wire 45, so as to transmit the alternating electrical signal generated by the electric field generator (not shown) to the dielectric element 413. One of the other three golden fingers 41120 of the wiring part 4112 has one end electrically connected to the ground terminal (not shown) of the temperature sensor 414 through the second conductive trace L2 connected thereto and the other two of the other three golden fingers 41120 each has one end respectively and electrically connected to the corresponding signal terminal (not shown) of the corresponding temperature sensors 414 through the third conductive traces L3, L3′ connected thereto. The other three golden fingers 41120 each has the other end respectively welded with the corresponding part of the wire 45. Thus, it realizes that the related signals detected by the temperature sensors 414 are transmitted to the electric field generator (not shown) quickly and concurrently through the second conductive trace L2, the third conductive traces L3, L3′ and the wire 45. Therefore, the alternating electrical signal or alternating current applied to the dielectric element 413 may be changed in time and quickly by the electric field generator (not shown) to achieve the purpose of avoiding cryogenic burns.

### An Alternate Implementation of the Fourth Embodiment of the Insulated Electrode

Referring to FIG. 31, the insulated electrode 400′ is an alternate implementation of the insulated electrode 400 of the fourth embodiment. The only difference between the insulated electrode 400′ and the insulated electrode 400 is that: the four corners of the backing 42′ of the insulated electrode 400′ are provided with concave corners 421′ recessed inward. Reference may be made to the fourth embodiment for the other contents. The backing 42′ is substantially arranged in a cross-shaped configuration. The concave corners 421′ communicate with the outside, and are in an “L” shape. When the insulated electrode 400′ is attached to the body surface corresponding to the patient's tumor site, the concave corner 421′ may prevent the corners of the backing 42′ from arching and forming wrinkles, and further avoid air entering between the electrode unit and the skin from the wrinkles, which results in cryogenic burns due to the increase of heat generation of the electrical functional component causing by the increase of the impedance between the electrical functional component and the skin.

Since the insulated electrodes 400, 400′ of the present embodiment use a separate electrode unit 410, it is easy to replace, and can be freely combined according to the size of the patient's tumor site, ensuring the effect of treating fields. Meanwhile, the flexible circuit board 411 of the insulated electrodes 400, 400′ of the present application is only provided with: a path of the first conductive trace L1 electrically connected with the dielectric element 413; a path of the second conductive trace L2 electrically connected with both the ground terminals (not shown) of the two temperature sensors 414; and two paths of the third conductive traces L3, L3′ electrically connected with the signal terminals (not shown) of the two temperature sensors 414 respectively, which realizes to transmit the alternating electrical signal of the electric field generator (not shown) to the dielectric element 413 through the first conductive trace L1, and realizes the purpose of applying the alternating electrical signal to the patient's tumor site for tumor treatment. At the same time, it realizes the signal transmission between the electric field generator (not shown) and the two temperature sensors 414 by electrically connecting the second conductive trace L2 and the third conductive trace L3, L3′ with the two temperature sensors 414, respectively. Thus, the difficulty of layering design is low, the structure is simple, the manufacturing process is simplified, the manufacturing is easy, and the product manufacturing yield is high, which may greatly reduce the manufacturing cost.

Furthermore, since the insulated electrodes 400, 400′ use a separate electrode unit 410 to apply an alternating electrical signal to the patient's tumor site, when it doesn't work properly, it only needs to replace the insulated electrodes 400, 400′ with the separate electrode unit 410 without needing to scrap the entire insulated electrode containing a plurality of electrode units 410, which can reduce the cost of tumor treatment for a patient. In addition, the insulated electrodes 400, 400′ of the present embodiment may be freely combined in quantity according to the patient's tumor site and the size of the patient's tumor site, ensuring the coverage area of the insulated electrodes 400, 400′ for tumor treating fields therapy and ensuring the required electric field strength of the tumor treating fields. Moreover, the relative positions of the plurality of insulated electrodes 400, 400′ may also be freely adjusted according to the patient's own physical differences, tumor site, and tumor size, in order to obtain the optimal electric field strength and coverage area for tumor treatment.

### A Fifth Embodiment of the Insulated Electrode

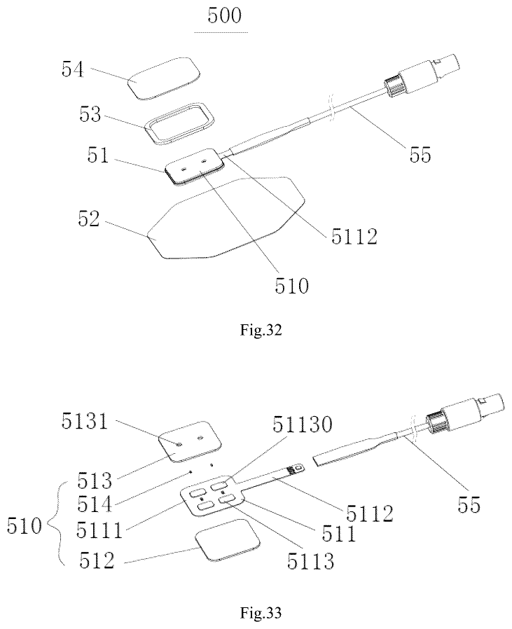

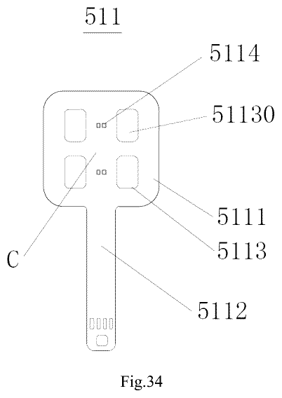

Referring to FIG. 32 to FIG. 34, the insulated electrode 500 of the present embodiment is similar to the insulated electrode 400 of the fourth embodiment. The insulated electrode 500 includes: a backing 52, an electrical functional component 51, a support member 53, an adhesive 54, and a wire 55. The electrical functional component 51 includes: a single electrode unit 510, and a wiring part 5112 connected to the electrode unit 510. The wiring part 5112 is welded and connected to the wire 55. The electrode unit 510 includes: a main body part 5111, an insulating plate 512, a dielectric element 513, and two temperature sensors 514. The main body part 5111 and the wiring part 5112 constitute a flexible circuit board 511. The only difference between the insulated electrode 500 of the present embodiment and the insulated electrode 400 of the fourth embodiment is that: the shape and dimension of the electrode unit 510 are different, and the shape, dimension, or arrangement of the two pairs of pads 5114 and the conductive pad 5113 correspondingly arranged on the main body part 5111 are different. The following only describes the differences, and other contents may refer to the fourth embodiment.

The electrode unit 510 is in a square sheet shape, and the main body part 5111, the insulating plate 512, and the dielectric element 513 are all in the structure of a square sheet with arc-shaped corners. The dimension of the main body part 5111 is approximately 32 mm×32 mm. The conductive pad 5113 of the main body part 5111 is substantially in a square structure, and has symmetry axes coincident with the symmetry axes of the main body part 5111. The conductive pad 5113 includes four conductive cores 51130 located at four corners and arranged at intervals. Each conductive core 51130 is in the structure of a rectangular with a dimension of approximately 9 mm×6 mm. Preferably, each conductive core 51130 is constructed in a rectangular shape with rounded corners. The longitudinal axis of each conductive core 51130 is parallel to the extension direction of the wiring part 5112.

The four conductive cores 51130 constituting the conductive pad 5113 are arranged in a matrix shape, and the four conductive cores 51130 are arranged in two rows and two columns. The gap between two columns of the conductive cores 51130 is about 8.5 mm, and the gap between two rows of the conductive cores 51130 is about 4 mm. The four conductive cores 51130 constituting the conductive pad 5113 are both center-symmetrically arranged and axial-symmetrically arranged, and each conductive core 51130 is also axial-symmetrically arranged, so that when the four conductive cores 51130 of the main body part 5111 are welded with the dielectric element 513, the stress balance of each welding point is guaranteed, improving the welding quality. Every two of the four conductive cores 51130 of the conductive pad 5113 are arranged at intervals, and a spacing C is formed between two adjacent conductive cores 51130. The four spacings C are connected and substantially arranged in a cross shape. Adjacent spacings C are communicated. An extension direction of two spacings C each located between two conductive cores 51130 in the same row is consistent with the extension direction of the wiring part 5112.

The two pairs of pads 5114 on the main body part 5111 are respectively located between the corresponding two conductive cores 51130 arranged at intervals in the row. The two pairs of pads 5114 are both located in the extension direction of the wiring part 5112. Each pair of the pads 5114 has a symmetrical center, and the line connecting the two symmetrical centers of the two pairs of pads 5114 is parallel to the extension direction of the wiring part 5112. The wiring part 5112 is equipped with four golden fingers 51120, which are electrically connected with the conductive pads 5113 and pads 5114 respectively through four conductive traces (not shown). The number and arrangement of the conductive traces are the same as the fourth embodiment, and will not be repeated.

In the present embodiment, since the insulated electrode 500 uses a separate electrode unit 510 to apply an alternating electrical signal to the patient's tumor site, when it doesn't work properly, it only needs to replace the insulated electrode 500 with the separate electrode unit 510 without needing to scrap the entire insulated electrode containing a plurality of electrode units 510, which can reduce the cost of tumor treatment for a patient. In addition, the insulated electrode 500 of the present embodiment may be freely combined in quantity according to the patient's tumor site and the size of the patient's tumor site, ensuring the coverage area of the insulated electrode 500 for tumor treating fields therapy and ensuring the required electric field strength of the tumor treating fields. Moreover, the relative positions of the plurality of insulated electrodes 500 may also be freely adjusted according to the patient's own physical differences, tumor site, and tumor size, in order to obtain the optimal electric field strength and coverage area for tumor treatment. At the same time, it may allow the skin on the patient's body surface to which the insulated electrode 500 is attached to breathe freely, avoiding the accumulation of heat on the patient's body surface caused by a long-term tumor treating fields, and not being able to dissipate in time, which causes sweating and clogging pores and results in skin inflammation.

### A Sixth Embodiment of the Insulated Electrode

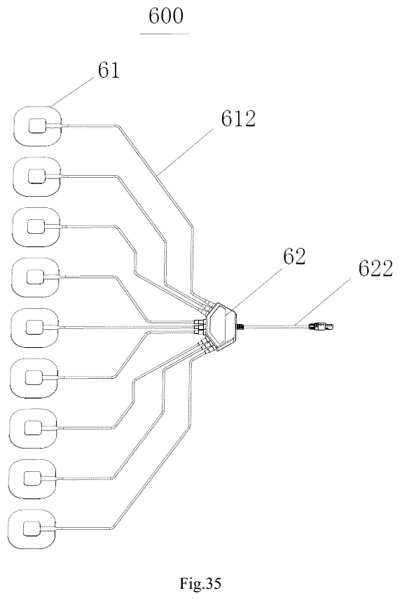

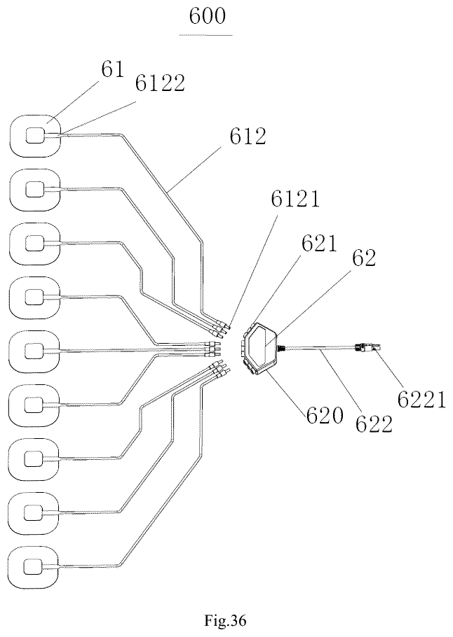

The insulated electrodes in the above third to fifth embodiments only have a single electrode unit, thus, during the treatment, a certain number of insulated electrodes are needed to ensure the therapeutic effect at the same time. However, for an electric field generator (not shown) or an adapter (not shown), the number of interfaces for connecting with insulated electrodes is limited. Therefore, referring to FIG. 35 and FIG. 36, the present application provides the following embodiments. In the present embodiment, the insulated electrode 600 includes a plurality of electrode sheets 61 and an electrical connector 62, and the plurality of electrode sheets 61 are detachably arranged on the electrical connector 62. The electrical connector 62 is electrically connected with an electric field generator (not shown) directly or through an adapter (not shown). The plurality of electrode sheets 61 are connected in parallel, so that if one of them is damaged, the use of other electrode sheets 61 will not be affected.

The electrode sheet 61 is provided with a first wire 612, and the first wire 612 has a first plug 6121 docked with the electrical connector 62. In the present embodiment, the specific structure of the electrode sheet 61 is basically the same as that of the insulated electrode 300 of the third embodiment, and the only difference is that: the insulated electrode 300 of the third embodiment is connected with an electric field generator (not shown) or an adapter (not shown), while the insulated electrode 600 of the present embodiment needs to be connected with the electric field generator (not shown) or the adapter (not shown) through an electrical connector 62, so the shape of the first plug 6121 of the first wire 612 is slightly different from that of the joint at the end of the wire 35 of the insulated electrode 300 of the third embodiment. Other structures of the electrode sheet 61 may refer to the related description of the insulated electrode 300 of the third embodiment, and will not be repeated here. The electrode sheet 61 may also use the insulated electrode 300 of the third embodiment, the insulated electrodes 400, 400′ of the fourth embodiment or the insulated electrode 500 of the fifth embodiment.

The electric connector 62 is provided with a plurality of sockets 621 and a second wire 622. The socket 621 is plugged with the first plug 6121 of the first wire 612 of the electrode sheet 61. The second wire 622 are provided with a second plug 6221 at an end thereof away from the electric connector 62, and the second plug 6221 can be directly plugged with an electric field generator (not shown), or can be first plugged with an adapter (not shown) and then plugged with an electric field generator (not shown) through the adapter (not shown), so as to realize the electrical connection with the electric field generator (not shown). The plurality of sockets 621 and the second wire 622 are respectively arranged at opposite ends of the electrical connector 62. The electrical connector 62 is plugged with the first plug 6121 of the first wire 612 of the electrode sheet 61 through the socket 621 thereof, so that the plurality of electrode sheets 61 are respectively connected to the electrical connector 62 to realize the electrical connection between the plurality of electrode sheets 61 and the electrical connector 62, and further realize the electrical connection between the plurality of electrode sheets 61 and the electric field generator through the second plug 6221 thereof plugged with the electric field generator (not shown) or the adapter (not shown). In use, the plurality of electrode sheets 61 are attached to the body surface corresponding to a patient's tumor site. The plurality of electrode sheets 61 are plugged into the corresponding sockets 621 of the electrical connector 62 through the first plugs 6121, and the electrical connector 62 is electrically connected with the electric field generator (not shown) through the second plugs 6221, so as to realize the transmission of the alternating electric signal generated by the electric field generator to the plurality of electrode sheets 61 through the electrical connector 62, and interfere or prevent the mitosis of the patient's tumor cells by attaching the plurality of electrode sheets 61 to the patient's tumor site, so as to achieve the purpose of treating the tumor.

In the present embodiment, the number of the sockets 621 of the electrical connector 62 is nine, and the number of the electrode sheets 61 is nine. The electrical connector 62 is equipped with a body 620, and the body 620 is substantially in a polyhedral structure. In the present embodiment, the body 620 is substantially in a hexagonal prism structure. The nine sockets 621 are respectively arranged on multiple adjacent sides of the body 620, and an obtuse angle is formed between adjacent sides. The second wire 622 is arranged on a side of the body 620 away from the sockets 621. In the present embodiment, the nine sockets 621 are evenly arranged on three adjacent sides of the body 620, and every three of the sockets 621 are arranged on a same side of the body 620 of the electrical connector 62. Terminals (not shown) inside the nine sockets 621 of the electrical connector 62 may be connected in series, so that the nine electrode sheets 61 are connected in series. Terminals (not shown) inside the nine sockets 621 of the electrical connector 62 may also be connected in parallel, so that the nine electrode sheets 61 are connected in parallel. When the terminals (not shown) inside the socket 621 of the electrical connector 62 are connected in series, all the electrode sheets 61 need to be plugged with the electrical connector 62 for use. When the terminals (not shown) inside the socket 621 of the electrical connector 62 are connected in parallel, it may select some of the electrode sheets 61 to be plugged with the electrical connector 62 as needed, which will be more convenient and flexible for use. Alternatively, the terminals (not shown) inside the nine sockets 621 of the electrical connector 62 may be partially connected in series and partially connected in parallel. The terminals (not shown) inside the socket 621 of the electrical connector 62 may be connected in series or parallel, or be partially connected in series and partially connected in parallel as needed, so that the plurality of electrode sheets 61 connected with the electrical connector 62 are all in series or all in parallel, or partially in series and partially in parallel. When the tumor is relatively large, it may select an appropriate number of electrode sheets 61 and freely adjust the spacing between the electrode sheets 61 as needed, so as to ensure the coverage area and the effect of treating fields of the insulated electrode 600 for tumor electric field treatment. When the tumor is located on a side of a site corresponding to the body, it may appropriately increase the number of electrode sheets 61 of the insulated electrode 600 adhered to the body surface corresponding to a side away from the tumor, so as to enhance the electric field strength on the side away from the tumor.

In the present embodiment, the electrode sheet 61 and the electrical connector 62 of the insulated electrode 600 are in a detachable plug-in arrangement. The electrode sheet 61 only includes one electrode unit 610, and each electrode unit 610 is electrically connected with the electric field generator (not shown) through the first wire 612 electrically connected with the electrode unit 610. When the electrode unit 610 or the first wire 612 is damaged and is unable to work, it only needs to replace the corresponding electrode sheet 61, which reduce the cost of tumor treatment for a patient. The electrode sheets 61 of the insulated electrode 600 may be freely combined in quantity according to the patient's tumor site and the tumor size, ensuring the coverage area of the insulated electrode 600 for tumor treating fields therapy and ensuring the electric field strength of the insulated electrode 600 for tumor treating fields therapy. Moreover, the relative positions of the plurality of electrode sheets 61 of the insulated electrodes 600 may also be freely adjusted according to the patient's own physical differences, tumor site, and tumor size, in order to obtain the optimal electric field strength and coverage area for tumor treatment. At the same time, it may allow the skin on the patient's body surface to which the electrode sheets 61 are attached to breathe freely, avoiding the accumulation of heat on the patient's body surface caused by a long-term tumor treating fields, and not being able to dissipate in time, which causes sweating and clogging pores and results in skin inflammation.

Insulated electrode 600′, an alternate implementation of the sixth embodiment

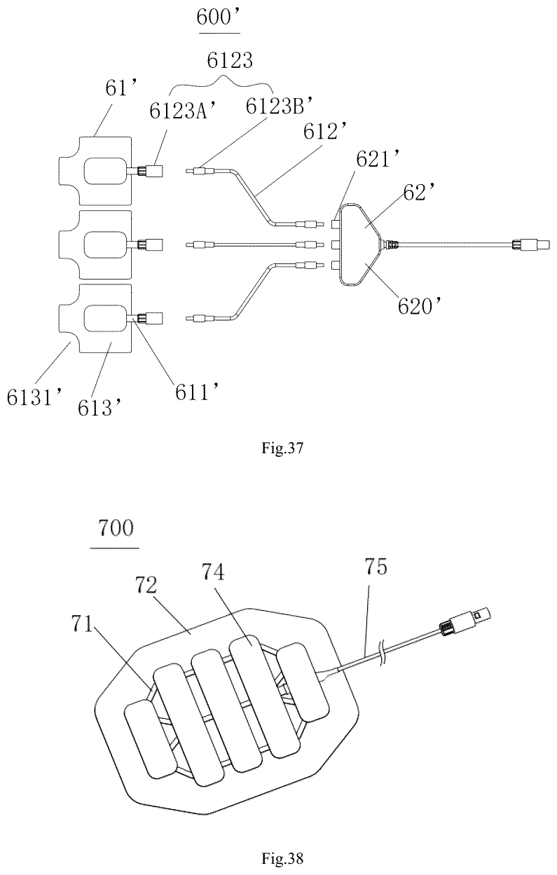

Referring to FIG. 37, the insulated electrode 600′ is an alternate implementation of the insulated electrode 600 of the above embodiment. The insulated electrode 600′ includes: a plurality of electrode sheets 61′ and an electrical connector 62′. The electrode sheets 61′ are electrically connected with the electrical connector 62′ in a pluggable manner, and the electrical connector 62′ is electrically connected with an adapter (not shown) or an electric field generator (not shown), so as to realize the electrical connection between the plurality of electrode sheets 61′ and the electric field generator (not shown).

The insulated electrode 600′ is basically the same as the insulated electrode 600, and the main differences are as follows: 1) the shape of the electrical connector 62′ and the number of sockets arranged are different; 2) the first wire 612′ of the electrode sheet 61′ is arranged in a pluggable manner; and 3) the shape of the electrode sheet 61′ is different.

The insulated electrode 600′ includes three electrode sheets 61′. The body 620′ of the electrical connector 62′ is substantially in a triangular prism structure. The electrical connector 62′ is arranged with three sockets 621′, and all the three sockets 621′ are arranged on the same side of the body 620′ of the electrical connector 62′. The wiring part 611′ of the electrode sheet 61′ is connected with the corresponding first wire 612′ in a detachable plug-in manner.

The wiring part 611′ of the electrode sheet 61′ and the first wire 612′ are electrically connected through a connector 6123′. The connector 6123′ includes a docking socket 6123A′ and a docking plug 6123B′. The docking socket 6123A′ is connected with the wiring part 611′, and the docking plug 6123B′ is connected with an end of the first wire 612′ away from the first plug 6121′. That is, the docking socket 6123A′ is arranged at the end of the wiring part 611′, and the docking plug 6123B′ is arranged at the end of the first wire 612′ away from the first plug 6121′. The docking socket 6123A′ and the electrode unit (not shown) are respectively located at opposite ends of the wiring part 611′. The docking plug 6123B′ and the first plug 6121′ are respectively arranged at opposite ends of the first wire 612′. When the electrode unit (not shown) of the electrode sheet 61′ is damaged and is unable to work, it may only replace the part of the electrode sheet 61′ except the first wire 612′, and the first wire 612′ can be continuously used, which further reducing the product scrap cost.

The electrode sheet 61′ of the present embodiment is basically the same as the insulated electrode 400 of the fourth embodiment, and the only differences are: (1) the docking socket 6123A′ located at the end of the wiring part 611′ is electrically connected with the connector 62′ through the docking plug 6123B′ and the first wire 612′, while the insulated electrode 400 of the fourth embodiment is electrically connected with the electric field generator (not shown) or the adapter (not shown), and thus, the shape of the docking socket 6123A′ is slightly different; and (2) the shape of the backing 613′ of the electrode sheet 61′ is slightly different. The backing 613′ is substantially structured in a shape of the Chinese character “ [symbol] ”, and has two concave corners 6131′ recessed inward from two corners thereof. The two concave corners 6131′ are located at two corners of the backing 613′ away from the wiring part 611′. Each concave corner 6131′ located at the corner of the backing 613′ communicates with the outside and is in an “L” shape. An angle between two sides of the backing 613′ that forms the concave corner 6131′ is greater than or equal to 90 degrees, so that when the electrode sheet 61′ is attached to the body surface corresponding to the patient's tumor site, it may prevent the corners of the backing 613′ from arching and forming wrinkles, and further avoid air entering between the electrode sheet 61′ and the skin from the wrinkles, which results in cryogenic burns due to the increase of heat generation of the electrode sheet 61′ causing by the increase of the impedance between the electrode sheet 61′ and the skin.

A plurality of electrode sheets 61, 61′ of the insulated electrode 600 and the insulated electrode 600′ are connected to the electrical connectors 62, 62′. It may be easier to replace the damaged electrode sheets 61, 61′ when a certain one of the electrode sheets 61, 61′ is damaged and is unable to work without needing to scrap all the plurality of electrode sheets 61, 61′, which may reduce the manufacturing cost and avoid waste, ensuring sufficient electric field strength during the tumor treating fields. Meanwhile, the plurality of electrode sheets 61, 61′ may be freely combined in quantity and freely adjusted in position according to the patient's body difference, tumor site and tumor size, ensuring the electric field strength applied to the patient's tumor site is optimal. In addition, the adhesion positions of the plurality of electrode sheets 61, 61′ and the spacings formed therebetween may also be freely adjusted according to the patient's own situation. It may allow the skin on the patient's tumor site to breathe freely, avoiding the rapid accumulation of heat on the patient's tumor site to which the electrode sheets 61, 61′ are adhered caused by a long-term treating field and not being able to dissipate in time, which causes the patient's body surface where the electrode sheets 61, 61′ are adhered sweating and clogging pores and results in skin inflammation.

### A Seventh Embodiment of the Insulated Electrode

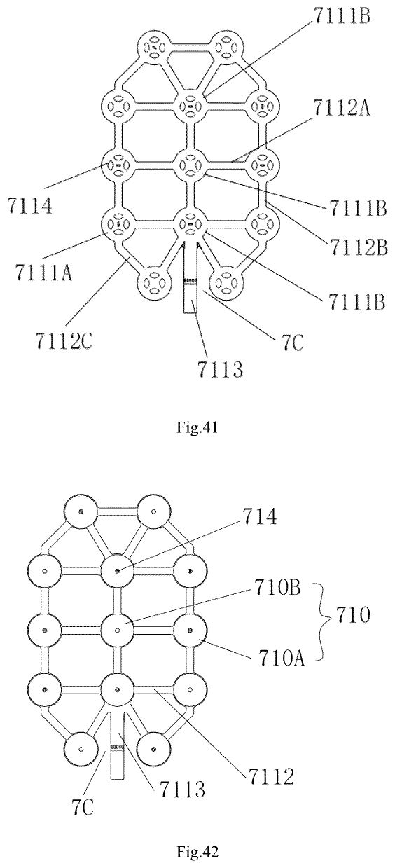

FIG. 38 to FIG. 42 illustrate an insulated electrode 700 according to the seventh embodiment of the present disclosure, which is attached to the body surface of a patient's trunk and used for tumor treating fields therapy to the tumor site located in the trunk. The insulated electrode 700 includes: a flexible backing 72; an electrical functional component 71 adhered to the backing 72; support members 73 adhered to the backing 72; adhesives 74 adhered to the support members 73; and a wire 75 electrically connected to the electrical functional component 71.

The electrical functional component 71 includes: a flexible circuit board 711; a plurality of insulating plates 712 and a plurality of dielectric elements 713 respectively arranged on opposite sides of the flexible circuit board 711; and a plurality of temperature sensors 714 fixed on the flexible circuit board 711. The temperature sensors 714 and the dielectric elements 713 are located on the same side of the flexible circuit board 711. The plurality of dielectric elements 713 are arranged on the side of the flexible circuit board 711 close to the patient's body surface, and the plurality of insulating plates 712 are arranged on the side of the flexible circuit board 711 away from the patient's body surface.

The flexible circuit board 711 includes: a plurality of main body parts 7111 arranged in an array; a plurality of connecting parts 7112 each located between adjacent main body parts 7111; and a wiring part 7113 electrically connected with the wires 75. The wiring part 7113 may be laterally extended from a connecting part 7112 or may be laterally extended from a main body part 7111 with one free end. The plurality of insulating plates 712 and the plurality of dielectric elements 713 are respectively arranged on opposite sides of the plurality of main body parts 7111 correspondingly. The main body parts 7111 are arranged at the ends of the connecting parts 7112, and each main body part 7111 is connected with at least two adjacent main body parts 7111 through the connecting parts 7112. Alternatively, the main body part 7111 may also be in a strip-shaped or band-shaped structure and integrally formed with the connecting part 7112.

The main body part 7111, the insulating plate 712 and the dielectric element 713 together constitute an electrode unit 710 of the electrical functional component 71. The arrangement of the electrode units 710 of the electrical functional component 71 is consistent with the arrangement of the main body parts 7111 of the flexible circuit board 711, and the connecting part 7112 is located between two adjacent electrode units 710. The structure of a single electrode unit 710 is the same as the structure of the electrode unit 110 of the insulated electrode 100 in the first embodiment. Reference may be made to the first embodiment for related contents.

The insulated electrode 700 of the present embodiment is different from the insulated electrode 100 of the first embodiment mainly in the arrangement form of the electrode units 710, which will be described in detail below.

In the present implementation, the number of the main body parts 7111 and the number of the dielectric elements 713 are both thirteen, and both of them may be distributed in a matrix area with five rows and three columns, or may be distributed in a matrix area with five rows and five columns. In view of the row arrangement, each of the first row and the last row is arranged with two main body parts 7111, and each of the middle three rows is arranged with three main body parts 7111. In the present embodiment, the main body parts 7111 are distributed in an array area of five rows and five columns. In view of the column arrangement, each of the first, the third and the fifth columns is arranged with three main body parts 7111, and each of the second and the fourth columns is arranged with two main body parts 7111. Specifically, the two main body parts 7111 in the first row are located in the second column and the fourth column respectively, the three main body parts 7111 in the middle three rows are located in the first column, the third column and the fifth column respectively, and the two main body parts 7111 in the last row are located in the second column and the fourth column respectively. Two adjacent main body parts 7111 in each row are arranged in interval columns. The spacings between two adjacent main body parts 7111 in the same row are equal. The spacings between two adjacent main body parts 7111 in the same column are equal. Two main body parts 7111 located in the last row are arranged in a disconnected state, and a spacing 7C is formed and located between the two main body parts 7111. The wiring part 7113 is laterally extended from the main body part 7111 located in the fourth row and the third column. The wiring part 7113 passes through the spacing 7C formed between the two main body parts 7111 in the last row.

The connecting part 7112 connects two adjacent main body parts 7111, and the conductive pad 7114 is arranged on a main body part 7111 located at the end of the connecting part 7112. The connecting parts 7112 includes: a plurality of first connecting part 7112A each connecting two adjacent main body parts 7111 located at interval columns of the same row; a plurality of second connecting part 7112B each connecting two main body parts 7111 located in adjacent rows of the same column; and a plurality of third connecting part 7112C each connecting two main body parts 7111 located in adjacent rows and adjacent columns and distributed diagonally. The first connecting parts 7112A are all respectively located between two adjacent main body parts 7111 in interval columns of each row, and have the same length. The second connecting parts 7112B are respectively located between two adjacent main body parts 7111 in each of the first column, the third column and the fifth column, and have the same length. The length of the third connecting part 7112C is greater than half of the length of the first connecting part 7112A. The length of the third connecting part 7112C is greater than the length of the second connecting part 7112B. Both the first connecting part 7112A and the second connecting part 7112B are substantially in a shape of the Chinese character “-”. The third connecting part 7112C is substantially in an “L” shape or in a shape of an inclined Chinese character “-”. There are eight third connecting parts 7112C, which are respectively located between the two main body parts 7111 in the second column of the first row and the second row of the first column, between the two main body parts 7111 in the second column of the first row and the third column of the second row, between the two main body parts 7111 in the third column of the second row and the fourth column of the first row, between the two main body parts 7111 in the fourth column of the first row and the fifth column of the second row, between the two main body parts 7111 in the second column of the last row and the fourth row of the first column, between the two main body parts 7111 in the second column of the last row and the third column of the fourth row, between the two main body parts 7111 in the third column of the fourth row and the fourth column of the last row, and between the two main body parts 7111 in the fourth column of the last row and the fifth column of the fourth row. Preferably, the length of the first connecting part 7112A is greater than the diameter of the main body part 7111. The length of the second connecting part 7112B is smaller than the diameter of the main body part 7111. The first connecting part 7112A and the second connecting part 7112B are vertically arranged. The third connecting part 7112C and its adjacent first connecting part 7112A are in an acute angle shape. The second connecting part 7112B and its adjacent third connecting part 7112C are also in an acute angle shape.

According to the distribution positions of the main body parts 7111 in the array, the main body parts 7111 can be divided into: peripheral main body parts 7111A located at the periphery of the array; and central main body parts 7111B surrounded by the peripheral main body parts 7111A and located at the inner layer of the array. Specifically, there are ten peripheral main body parts 7111A, and there are three central main body parts 7111B in the same column. Every peripheral main body part 7111A is connected with the corresponding central main body part 7111B by a connecting part 7112. Two adjacent peripheral main body parts 7111A are electrically connected through either the first connecting part 7112A, the second connecting part 7112B or the third connecting part 7112C. Specifically, two adjacent peripheral main body parts 7111A located in the same column are connected by the second connecting part 7112B, two adjacent peripheral main body parts 7111A located in the same row are connected by the first connecting part 7112A, and two adjacent peripheral main body parts 7111A located in adjacent rows and adjacent columns and arranged diagonally are connected by the third connecting part 7112C. The peripheral main body parts 7111A and the first connecting parts 7112A, the second connecting parts 7112B and the third connecting parts 7112C located between two adjacent peripheral main body parts 7111A are substantially in an octagonal shape with one end opening. The peripheral main body parts 7111A are axial-symmetrically arranged, and have a symmetry axis coincides with the straight line at which the three central main body parts 7111B are located.

The central main body parts 7111B are the three main body parts 7111 located in the third column. Each central main body part 7111B is connected to the peripheral main body part 7111A adjacent thereto by a first connecting part 7112A or a third connecting part 7112C. Two adjacent central main body parts 7111B are electrically connected by a second connecting part 7112B. Specifically, the central main body part 7111B is electrically connected with the peripheral main body part 7111A located in the same row and adjacent thereto through the first connecting part 7112A, and the central main body part 7111B is electrically connected with the peripheral main body part 7111A diagonally located in adjacent rows and adjacent columns and adjcent thereto through the third connecting part 7112C. Thus, each central main body part 7111B is connected to at least two peripheral main body parts 7111A adjacent thereto by the corresponding connecting parts 7112, ensuring a relative fixed position and stable connection between the peripheral main body parts 7111A and the central main body parts 7111B, which facilitates to weld the dielectric elements 713 on the flexible circuit board 711. That is, the central main body part 7111B located in the third row is connected with the peripheral main body parts 7111A located in the same row only through the first connecting parts 7112A, and is arranged in a disconnected state with the peripheral main body parts 7111A adjcent there to and located in adjacent rows and adjacent columns and arranged diagonally. Each of the other two central main body parts 7111B is not only connected with the peripheral main body part 7111A located in adjacent rows and columns and arranged diagonally through the third connecting part 7112C, but is also connected with the peripheral main body parts 7111A located in the same row and adjcent thereto through the first connecting parts 7112A.

The wiring part 7113 is laterally extended from one end of one of the two main body parts 7111 located in the third column. Specifically, the wiring part 7113 is laterally extended from the main body part 7111 in the third column of the fourth row. The wiring part 7113 is extended from the central body portion 7111B located at the end toward where away from an area the array of the main body parts 7111 is located. The wiring part 7113 is located between two third connecting parts 7112C, and is connected to the central main body part 7111B located at the end together with the two third connecting parts 7112C. The wiring part 7113 and the two third connecting parts 7112C, which are connected to the same central main body part 7111B together with the wiring part 7113, are substantially in an arrow shape. The wiring part 7113 extends between two peripheral main body parts 7111A which are located in the same row and arranged in a disconnected state. The wiring part 7113 and the first connecting part 7112A are substantially arranged vertically. The wiring part 7113 and the second connecting part 7112B are substantially arranged in parallel. The wiring part 7113 is substantially in a shape of the Chinese character “-”. An angle between the wiring part 7113 and the third connecting part 7112C which are connected with the same main body part 7111 is an acute angle. In other embodiments, the wiring part 7113 may also be laterally extended from the central main body part 7111B or the main body part 7111 located in the third column of the second row. The two main body parts 7111 located in the first row are arranged in a disconnected state, and the wiring part 7113 passes through the spacing between that two main body parts 7111. In other embodiments, the wiring part 7113 may also be laterally extended from a second connecting part 7112B located between two adjacent central main body parts 7111B, and the wiring part 7113 and the second connecting part 7112B are vertically arranged. The wiring part 7113 and the second connecting part 7112B from which the wiring part 7113 is extended are substantially in a “T” shape.

The temperature sensor 714 is fixed on the main body part 7111, and is used to monitor the temperature of the adhesive 74, thereby monitoring the temperature of the human skin to which the adhesive 74 is adhered. The arrangement form of the above main body parts 7111 is the arrangement form of the electrode units 710. That is, the thirteen electrode units 710 are arranged in five rows and five columns as described above, including one central electrode unit 710B located in the middle column (i.e., the third column) of the middle row (i.e., the third row), and the other twelve peripheral electrode units 710A. In the present embodiment, there are eight temperature sensors 714, which are selectively arranged on the peripheral electrode units 710A.

The support member 73 is in a sheet shape. A plurality of support members 73 are provided. The support members 73 are adhered to the backing 72 in a manner that surrounding the electrode units 710 arranged in rows. The plurality of support members 73 are arranged at intervals. The support member 73 has a plurality of through holes 731 arranged correspondingly to the corresponding electrode units 710. The plurality of through holes 731 are arranged at intervals. The thickness of the support member 73 is basically the same as the thickness of the electrode unit 710, and the plane of the top end of the support member 73 is at the same vertical height as the surface of the side of the electrode unit 710 facing the patient's body surface. That is, the surface of the side of the support member 73 close to the patient's body surface is flush with the surface of the side of the dielectric element 713 close to the patient's body surface, so that the adhesive 74 may be evenly covered on the support member 73 and the electrode unit 710, improving the comfort of applying the insulated electrode 700. The support member 73 may be made of polyethylene (PE), or PET, or thermal conductive silica gel sheet, or a soft, chemically stable, light weight, non-deformable and non-toxic insulating material composed of polyurethane, polyethylene, dispersant, flame retardant and carbon fiber. Preferably, the support member 73 is made of flexible foam.

The adhesive 74 is in a sheet shape, and has one side attached to the support member 73 and the dielectric element 713 and the other side attached to a patient's body surface. The adhesive 74 is made of conductive hydrogel, and can be used as a conductive medium to conduct the alternating current passing through the dielectric element 713 to the patient's tumor site. The number of the adhesives 74 is the same as the number of the support members 73. The size of the adhesive 74 is approximately the same as the size of the support member 73.

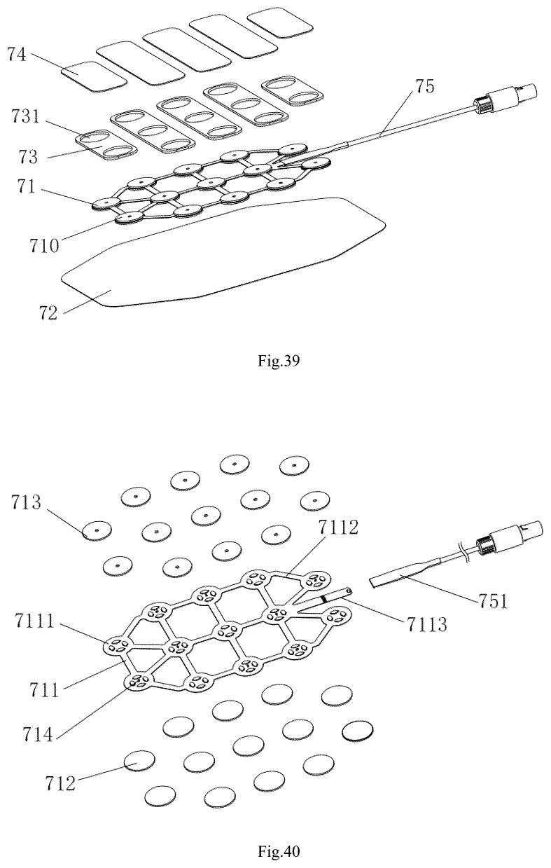

With reference to FIG. 39, the backing 72 is in a sheet shape, which is mainly made of materials that are compatible with flexibility, permeability, insulation, and sterilization. The backing 72 has a plurality of permeable holes (not shown) arranged therethrough for allowing the hair follicles and sweat glands of the skin of the patient's body surface covered by the backing 72 to breathe freely when the backing 72 is adhered to a patient's body surface, preventing the sweat glands and hair follicles of the patient's body surface covered by the backing 72 from blockage, which otherwise leads to damage to the superficial layer of the patient's skin and cause skin inflammation. The backing 72 is a mesh fabric. Specifically, the backing 72 is a mesh non-woven fabric. The side of the backing 72 facing the patient's body surface is also coated with biocompatible adhesive (not shown), which is used to tightly adhere the backing 72 to the body surface of the patient's target area.

### An Alternate Embodiment 1 of the Seventh Embodiment of the Insulated Electrode

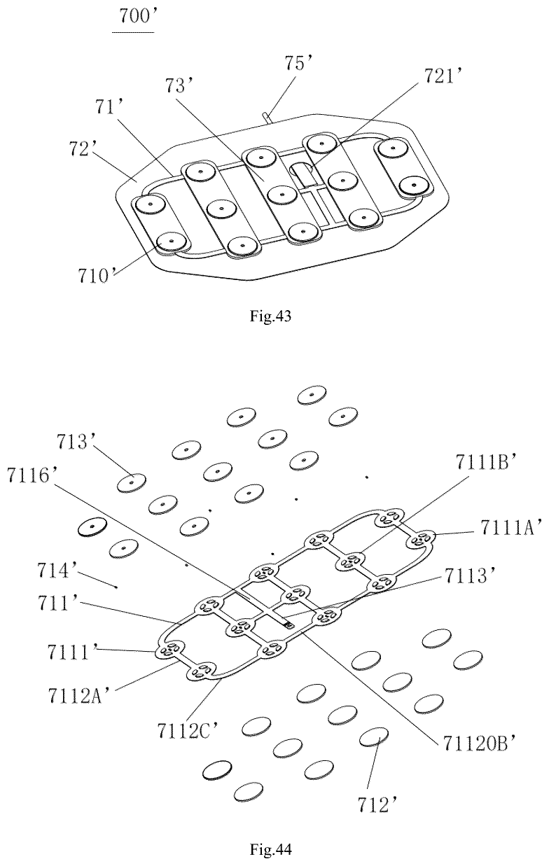

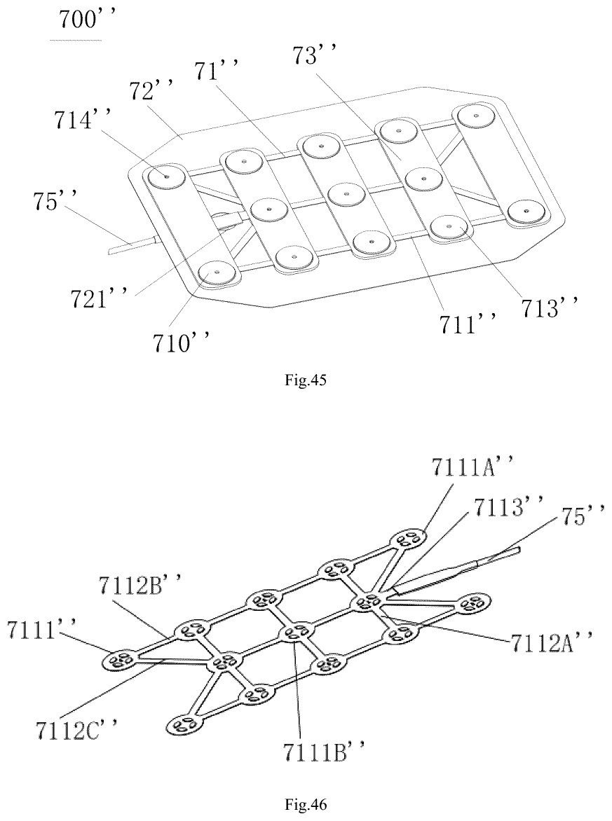

FIG. 43 to FIG. 44 illustrate an alternate embodiment 1 of the seventh embodiment of the insulated electrode, the insulated electrode 700′, which is also attached to the body surface of a patient's trunk and used in the tumor treating fields therapy at a tumor site located on the trunk. The difference between the insulated electrode 700′ and the insulated electrode 700 of the seventh embodiment is that: every two of the peripheral main body parts 7111A′ of the flexible circuit board 711′ of the electrical functional component 71′ of the insulated electrode 700′ are connected through the connecting part, and each central main body part 7111B′ is only connected with the peripheral main body parts 7111A′ adjacent there to and located in the same row. Specifically, two adjacent peripheral main body parts 7111A′ are connected by a first 20 connecting part 7112A′, a second connecting part 7112B′, or a third connecting part 712C′. The peripheral main body parts 7111A′, as well as the first connecting parts 7112A′, the second connecting part 7112B′ and the third connecting part 7112C′ located between two adjacent peripheral main body parts 7111A′ are substantially structured in a racetrack shape. The central main body part 7111B′ is connected with the peripheral main body parts 7111A′ located in the same row through the first connecting parts 7112A′. The central main body part 7111B′ and the peripheral main body parts 7111A′ located in adjacent rows and adjacent columns and arranged diagonally are arranged in a disconnected state. Two adjacent central main body parts 7111B′ of the three central main body parts 7111B′ are arranged in a disconnected state. There is no second connecting part 7112B′ arranged between the two adjacent central main body parts 7111B′ arranged in a disconnected state. The third connecting part 7112C′ is in an arc shape. There are four third connecting parts 7112C′, which are respectively located between the two peripheral main body parts 7111A′ in the second column of the first row and the second row of the first column, between the two peripheral main body parts 7111A′ in the fourth column of the first row and the fifth column of the second row, between the two peripheral main body parts 7111A′ in the second column of the last row and the fourth row of the first column, and between the two peripheral main body part 7111A′ in the fourth column of the last row and the 5 fifth column of the forth row. The third connecting part 7112C′ and the first connecting part 7112A′ adjacent thereto are substantially in an obtuse angle shape or an acute angle shape. The third connecting part 7112C′ and the second connecting part 7112B′ adjacent thereto are substantially in an obtuse angle shape. The diameter of the peripheral main body part 7111A′ is the same as that of the central main body part 7111B′, and the length of the second connecting part 7112B′ is slightly larger than the diameter of the peripheral main body part 7111A′.

The wiring part 7113′ is laterally extended from the second connecting part 7112B′. Specifically, the wiring part 7113′ is laterally extended form the second connecting part 7112B′ located between two adjacent central main body parts 7111B′. The wiring part 7113′ and the second connecting part 7112B′ from which the wiring part 7113′ extends are substantially in a “T” shape. The wiring part 7113′ and the second connecting part 7112B′ are vertically arranged. The wiring part 7113′ and the first connecting part 7112A′ are substantially arranged in parallel.

The flexible circuit board 711′ further includes a reinforcing part 7116′ arranged opposite to the wiring part 7113′, which may provide traction for the wiring part 7113′ and prevent the application of the insulated electrode 700′ from being affected by uneven force when the insulated electrode 700′ is adhered to the body surface of a patient's tumor site. Specifically, the reinforcing part 7116′ is extended from the second connecting part 7112B′ from which the wiring part 7113′ is laterally extended. The reinforcing part 7116′ and the wiring part 7113′ are respectively located on the opposite sides of the second connecting part 7112B′ connected with the wiring part 7113′. One end of the reinforcing part 7116′ is connected to the second connecting part 7112B′ connected with the wiring part 7113′, and the other end of the reinforcing part 7116′ is connected to the second connecting part 7112B′ adjacent to the second connecting part 7112B′ and located between two adjacent peripheral main body parts 7111A′. The reinforcing part 7116′ is bridged between two adjacent second connecting parts 7112B′ that are arranged in parallel. The reinforcing part 7116′, the wiring part 7113′ and the second connecting part 7112B′ connected with the wiring part 7113′ are substantially in a cross shape.

The temperature sensor 714′ may be selectively arranged on the peripheral main body part 7111A′ and the central main body part 7111B′.

The backing 72′ is also provided with a threading hole 721′ corresponding to the wiring part 7113′ of the flexible circuit board 711′. An end of the wire 75′ passes through the threading hole 721′ and is electrically connected with the wiring part 7113′. The wire 75′ extends into the flexible circuit board 711′ from one side of the backing 72′ and is connected with the wiring part 7113′, so as to prevent a large number of wires 75′ from being directly pressed on a patient's epidermis, which leads to the decrease of comfort when the insulated electrode 700′ is attached.

### An Alternate Embodiment 2 of the Seventh Embodiment of the Insulated Electrode

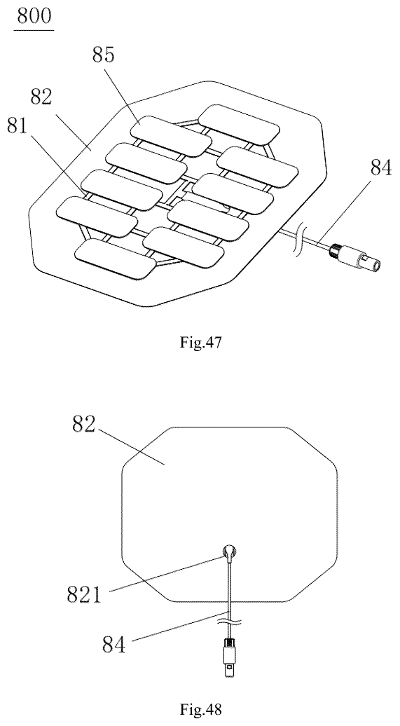

FIG. 45 to FIG. 46 illustrate the insulated electrode 700″ which is an alternate embodiment 2 of the seventh embodiment of the insulated electrode 700. The inslulated electrode 700″ is also attached to the body surface of a patient's trunk and used in the tumor treating fields therapy at a tumor site located on the trunk.

The difference between the insulated electrode 700″ of the present embodiment and the insulated electrode 700 of the seventh embodiment is that: the arrangement of the main body parts 7111″ of the flexible circuit board 711″ of the electrical functional component 71″ of the insulated electrode 700″ is different when arranged in an array area of five rows and three columns. In view of the column arrangement, the first column and the third column are respectively provided with five main body parts 7111″, and the second column is provided with three main body parts 7111″. Specifically, the two main body parts 7111″ located in the first row are respectively located in the first column and the third column. The two main body parts 7111″ located in the last row are also respectively located in the first column and the third column. The three main body parts 7111″ in each of the middle three rows are respectively located in the first column, the second column and the third column. The main body parts 7111″ located in the first row and the last row are arranged in interval columns, and the main body parts 7111″ located in the first row and the last row are arranged in a disconnected state. The spacings between two adjacent main body parts 7111″ in the same row are unequal. The spacings between two adjacent main body parts 7111″ in the same column are equal. The thirteen main body parts 7111″ are axial-symmetrically arranged, and one of the symmetry axes is coincident with the straight line at which the three main body parts 7111″ of the third row is located, and the other symmetry axis is coincident with the straight line at which the three main body parts 7111″ of the second column is located. The thirteen main body parts 7111″ are also center-symmetrically arranged, and the symmetry center is coincident with the center of the main body part 7111″ located in the third row and the third column. The electrode units 710″ are arranged consistently with the main body parts 7111″, and are located in an array area of five rows and three columns.

According to the distribution positions of the main body parts 7111″ in the array, the main body parts 7111″ can be divided into twelve peripheral main body parts 7111A″ located at the periphery of the array and one central main body part 7111B″ surrounded by the peripheral main body parts 7111A″ and located at the inner layer of the array. Specifically, the central main body part 7111B″ is the main body part 7111″ located at the third row and the second column. The twelve peripheral main body parts 7111A″ are all the other main body parts 7111″ except the main body part 7111″ located in the third row and the second column. The peripheral main body parts 7111A″ are connected by either the second connecting part 7112B″ or the third connecting part 7112C″. Two adjacent peripheral main body parts 7111A″ in the same column are all connected by the second connecting part 7112B″. Two peripheral main body parts 7111A″ located in adjacent rows and adjacent columns and arranged diagonally are connected by the third connecting part 7112C″. Two adjacent peripheral main body parts 7111A″ arranged at interval columns and located in the same row are arranged in a disconnected state. The peripheral main body part 7111A″ and the central main body part 7111B″ are either connected by a first connecting part 7112A″ or are connected by a second connecting part 7112B″. Specifically, the peripheral main body part 7111A″ and the central main body part 7111B″ adjacent to each other and located in the same row are connected by the first connecting part 7112A″. The peripheral main body part 7111A″ and the central main body part 7111B″ adjacent to each other and located in the same column are connected by the second connecting part 7112B″. The first connecting part 7112A″ is located between two main body parts 7111″ in adjacent columns of the same row, and has the same length. The second connecting part 7112B″ is located between two main body parts 7111″ in adjacent rows of the same column, and has the same length. The length of the third connecting part 7112C″ is greater than the length of the first connecting part 7112A″. There are four third connecting parts 7112C″, which are respectively located between the two peripheral main body parts 7111A″ in the first column of the first row and the second column of the second row, between the two peripheral main body parts 7111A″ in the second column of the second row and the third column of the first row, between the two peripheral main body parts 7111A″ in the first column of the fifth row and the second column of the fourth row, and between the two peripheral main body parts 7111A″ in the second column of the fourth row and the third column of the fifth row. The peripheral main body parts 7111A″ are axial-symmetrically arranged, and one of the symmetry axes is coincident with the extension direction of the row at which the central main body part 7111B″ is located, and the other symmetry axis is coincident with the extension direction of the column at which the central main body part 7111B″ is located. The wiring part 7113″ is extended from the peripheral main body part 7111A″ located in the second column of the fourth row. The wiring part 7113″ is located between two adjacent third connecting parts 7112C″ that are jointly connected to the same peripheral main body part 7111A″ with the wiring part 7113″. The temperature sensors 714″ are selectively arranged on the peripheral main body parts 7111A″.

The backing 72″ is also provided with a threading hole 721″ corresponding to the wiring part 7113″ of the flexible circuit board 711″. An end of the wire 75″ passes through the threading hole 721″ and is electrically connected with the wiring part 7113″. The wire 75″ extends into the flexible circuit board 711″ from one side of the backing 72″ and is connected with the wiring part 7113″, so as to prevent a large number of wires 75′ from being directly pressed on a patient's epidermis, which leads to the decrease of comfort when the insulated electrode 700″ is attached.

### An Eighth Embodiment of the Insulated Electrode

Referring to FIG. 47 to FIG. 51, the insulated electrode 800 includes: a backing 82; an electrical functional component 81 adhered to the backing 82; a support member 83 adhered to the backing 82; an adhesive 85 covering the support member 83 and the corresponding parts of the electrical functional component 81; and a wire 84 electrically connected to the electrical functional component 81. The insulated electrode 800 is attached to the body surface corresponding to a patient's tumor site through the backing 82, and an alternating electric field is applied to the patient's tumor site through the electrical functional component 81 to interfere or prevent the mitosis of the patient's tumor cells, so as to achieve the purpose of treating the tumor.

The electrical functional component 81 is in a grid shape, and includes: a plurality of electrode units 810 arranged in an array, a plurality of connecting parts 8112 each connecting two adjacent electrode units 810; and a wiring part 8113 welded to a wire 84. The plurality of electrode units 810 are distributed at intervals on the grid points of the electrical functional component 81. Each electrode unit 810 is connected to at least two adjacent electrode units 810 through connecting parts 8112. Each electrode unit 810 is at least connected to two connecting parts 8112. There are at least ten electrode units 810, and are distributed in an array area of at least three rows and four columns, which may increase the coverage area of the electrode units 810 of the insulated electrode 800, enhance the electric field intensity applied to the tumor site for tumor treating fields treatment, increase the coverage range of the alternating electric field to the tumor site, and improve the therapeutic effect.

Preferably, each electrode unit 810 is connected with at least three adjacent electrode units 810 through the connecting parts 8112. Each electrode unit 810 is connected to at least three connecting parts 8112. There are twenty electrode units 810, and are distributed in an array area of four rows and six columns. The number of electrode units 810 in each column is not exactly the same. The number of electrode units 810 in each row may be the same, or may not be exactly the same. At least two adjacent electrode units 810 among the plurality of electrode units 810 are arranged in a disconnected state, and a spacing 8C is formed between the adjacent two electrode units 810 arranged in a disconnected state and is for the wiring part 8113 to pass through. The wiring part 8113 is laterally extended from the connecting part 8112 opposite to the spacing 8C. The wiring part 8113 and the connecting part 8112 from which the wiring part 8113 is extended are vertically arranged, and are substantially in a “T” shape. The wiring part 8113 is substantially in a shape of the Chinese character “-”. Alternatively, the wiring part 8113 is in a “T” shape, and is bridged between two connecting parts 8112 respectively connected with the two adjacent electrode units 810 arranged in a disconnected state. The wiring part 8113 is located among the plurality of electrode units 810, and is arranged in a space surrounded by the plurality of electrode units 810, so as to prevent the overall dimension of the electrical functional component 81 from being too large, resulting in an increase in manufacturing cost.

Twenty electrode units 810 are arranged in an array area of four rows and six columns in such a way that two electrode units 810 are arranged in each column of two columns and four electrode units 810 are arranged in each column of the other four columns. Specifically, the twenty electrode units 810 are distributed in the array area of four rows and six columns in such a way that four columns with four electrode units 810 are adjacent to each other. The spacings between two adjacent electrode units 810 arranged in rows are the same. A plurality of connecting parts 8112 connecting two adjacent electrode units 810 arranged in rows have the same length. Specifically, the electrode units 810 in each of the two columns provided with only two electrode units 810 are arranged in adjacent rows. The spacings between the two adjacent electrode units 810 arranged in columns are the same. A plurality of connecting parts 8112 connecting two adjacent electrode units 810 arranged in columns have the same length. The four electrode units 810 in that two columns may be respectively arranged in a row-aligned manner, or may be respectively arranged in a staggered manner in the row direction, or may also be arranged in a way that one of them is in a row-aligned manner and the other are in a staggered manner in the row direction. Alternatively, two electrode units 810 of at least one of the two columns provided with only two electrode units 810 are arranged in interval rows. The spacings between the electrode units 810 arranged in columns are different. A plurality of connecting parts 8112 connecting two adjacent electrode units 810 arranged in columns have different lengths.

Alternatively, the twenty electrode units 810 are arranged in an array area of four rows and six columns in such a way that at least two columns of the four columns that provided with four electrode units 810 are arranged at intervals. The spacings between two adjacent electrode units 810 arranged in rows are different. A plurality of connecting parts 8112 connecting two adjacent electrode units 810 arranged in rows have different lengths. Specifically, the electrode units 810 in at least one column of the two columns provided with only two electrode units 810 are arranged at intervals. The spacings between the two adjacent electrode units 810 arranged in columns are different. A plurality of connecting parts 8112 connecting two adjacent electrode units 810 arranged in columns have different lengths. Alternatively, the two electrode units 810 of each column of the two columns provided with only two electrode units 810 are arranged in adjacent rows, the spacings between the two adjacent electrode units 810 arranged in columns are the same, and the plurality of connecting parts 8112 connecting two adjacent electrode units 810 arranged in columns have the same length.

Alternatively, the twenty electrode units 810 are arranged in an array area of four rows and six columns in such a way that: one electrode unit 810 is arranged in one column, three electrode units 810 are arranged in one column, and four electrode units 810 are arranged in each of the other four columns. Specifically, the twenty electrode units 810 are arranged in an array area of four rows and six columns in such a way that: one electrode unit 810 is arranged in the first column, four electrode units 810 are arranged in each column of the middle four columns, and three electrode units 810 adjacent to each other are arranged in the last column. The spacings between two adjacent electrode units 810 arranged in rows are the same, and the spacings between two adjacent electrode units 810 arranged in columns are the same. That is, a plurality of connecting parts 8112 connecting two adjacent electrode units 810 in the same row have the same length, and a plurality of connecting parts 8112 connecting two adjacent electrode units 810 in the same column have the same length.

Alternatively, the twenty electrode units 810 are arranged in an array area of four rows and six columns in such a way that: one electrode unit 810 is arranged in the first column, four electrode units 810 are arranged in each column of the middle four columns, and three electrode units 810 are arranged in the last column with adjacent two electrode units 810 being arranged in interval rows. The spacings between two adjacent electrode units 810 arranged in rows are the same, and the spacings between two adjacent electrode units 810 arranged in columns are different. That is, a plurality of connecting parts 8112 connecting two adjacent electrode units 810 in the same row have the same length, and a plurality of connecting parts 8112 connecting two adjacent electrode units 810 in the same column have different lengths.

Alternatively, the twenty electrode units are arranged in an array area of four rows and six columns in such a way that: four electrode units 810 are arranged in each of the first to the four columns, three electrode units 810 are arranged in the fifth column, and only one electrode unit 810 is arranged in the last column. The electrode unit 810 in the last column and one of the three electrode units 810 in the fifth column are arranged in a row-aligned manner, and the three electrode units 810 in the fifth column are arranged adjacently in the row direction. The spacings between two adjacent electrode units 810 arranged in rows are the same, and the spacings between two adjacent electrode units 810 arranged in columns are the same. A plurality of connecting parts 8112 connecting two adjacent electrode units 810 arranged in rows have the same length, and a plurality of connecting parts 8112 connecting two adjacent electrode units 810 arranged in columns have the same length. Alternatively, the electrode units 810 in the last column and the three electrode units 810 in the fifth column are arranged in a staggered manner in the row direction, and the three electrode units 810 in the fifth column are arranged adjacently in the row direction. The spacings between two adjacent electrode units 810 arranged in rows are different, and the spacings between two adjacent electrode units 810 arranged in columns are the same. A plurality of connecting parts 8112 connecting two adjacent electrode units 810 arranged in rows have different lengths, and a plurality of connecting parts 8112 connecting two adjacent electrode units 810 arranged in columns have the same length. Alternatively, the electrode unit 810 in the last column and the three electrode units 810 in the fifth column are staggered in the row direction, and the adjacent two electrode units 810 of the three electrode units 810 in the fifth column are arranged in interval rows. The spacings between two adjacent electrode units 810 arranged in rows are different, and the spacings between two adjacent electrode units 810 arranged in columns are different. A plurality of connecting parts 8112 connecting two adjacent electrode units 810 arranged in rows have different lengths, and a plurality of connecting parts 8112 connecting two adjacent electrode units 810 arranged in columns have different lengths.

Alternatively, the twenty electrode units 810 are arranged in an array area of four rows and six columns in such a way that: at least two columns of the four columns that provided with four electrode units 810 in each column are arranged in interval columns. The spacings between two adjacent electrode units 810 arranged in rows are different. A plurality of connecting parts 8112 connecting two adjacent electrode units 810 arranged in rows have different lengths. The spacings between two adjacent electrode units 810 arranged in columns may either be the same or different. A plurality of connecting parts 8112 connecting two adjacent electrode units 810 arranged in columns may either have the same length or different lengths.

The twenty electrode units 810 of the present embodiment are arranged in an array area of four rows and six columns in such a way that: four electrode units 810 are arranged in each of the first row and the last row, and six electrode units 810 are arranged in each of the middle two rows. In view of the column arrangement, each of the first and the sixth columns is provided with two electrode units 810, and each of the middle four columns is provided with four electrode units 810. The electrode units 810 of the first column and the sixth column located in the same column are distributed adjacently in the row direction, and the electrode units 810 of the fist and sixth columns are respectively arranged in a row-aligned manner. Specifically, the four electrode units 810 in the first row are respectively located in the columns from the second column to the fifth column, the six electrode units 810 in each of the middle two rows are respectively located in the columns from the first column to the sixth column, and the four electrode units 810 in the last row are respectively located in the columns from the second column to the fifth column. A plurality of electrode units 810 of the electrical function component 81 are axial-symmetrically arranged. A plurality of electrode units 810 of the electrical function component 81 are both axial-symmetrically arranged in the row direction and axial-symmetrically arranged in the column direction. The twenty electrode units 810 are in an octagonal shape.

The connecting parts 8112 connect all two adjacent electrode units 810 located at the periphery of the array. Among the two adjacent electrode units 810 located at the inner layer of the array, at least one pair of the two adjacent electrode units 810 is arranged in a disconnected state. Specifically, the connecting parts 8112 are arranged between all adjacent two electrode units 810 except the two electrode units 810 located in the third column of the second row and the fourth column of the second row, and the two electrode units 810 located in the third column of the third row and the fourth column of the third row. The lengths of the connecting parts 8112 connecting two adjacent electrode units 810 arranged in rows are equal. The lengths of the connecting parts 8112 connecting two adjacent electrode units 810 arranged in columns are equal. The connecting parts 8112 are located between two adjacent electrode units 810 arranged in rows, between two electrode units 810 arranged in columns, and between two adjacent electrode units 810 arranged in adjacent rows and adjacent columns and arranged diagonally and at the periphery of the array.

The spacings 8C are arranged between two adjacent electrode units 810 located in the third column of the second row and the fourth column of the second row, and between two adjacent electrode units 810 located in the third column of the third row and the fourth column of the third row. The wiring part 8113 is located between the electrode units 810 of the third column and the electrode units 810 of the fourth column. The wiring part 8113 is substantially in a “T” shape, which passes through the spacing 8C and bridges the connecting part 8112 located between two adjacent electrode units 810 in the middle of the third column and the connecting part 8112 located between two adjacent electrode units 810 in the middle of the fourth column. The wiring part 8113 and two adjacent connecting parts 8112 connected thereto are axial-symmetrically arranged. Alternatively, the wiring part 8113 is in a shape of the Chinese character “-”, and is laterally extended from the connecting part 8112 corresponding to the spacing 8C toward the spacing 8C.

The wiring part 8113 of the electrical function component 81 is electrically connected to the wire 84. In the present implementation, rows of golden fingers 81130 welded to the wires 84 are arranged in a staggered manner, separately, on two side surfaces of an end of the wiring part 8113 away from the connecting part 8112 to which the wiring part 8113 is connected. One end of the wire 84 is electrically connected to the golden fingers 81130 of the wiring part 8113, and the other end of the wire 84 is electrically connected to the electric field generator (not shown) through the arranged plug 42, so as to provide the insulated electrode 800 with alternating current signals for tumor treatment during the tumor treating fields therapy. The periphery of the welding point of the wire 84 and the golden fingers 81130 of the wiring part 8113 is covered with a heat shrinkable sleeve 81. The heat shrinkable sleeve 81 provides support and insulation protection for the connecting point between the wire 84 and the wiring part 8113 of the electrical functional component 81, so as to prevent the connecting point between the wire 84 and the wiring part 8113 of the electrical functional component 81 from being broken. Meanwhile, it may also be dustproof and waterproof.

The electrode unit 810 includes: a main body part 8111 arranged at two opposite ends of the connecting part 8112; an insulating plate 812 arranged at one side of the main body part 8111 away from human skin; a dielectric element 813 arranged at one side of the main body part 8111 facing human skin; and a temperature sensor 814 selectively arranged on the main body part 8111 and located at the same side as the dielectric element 813. The specific structure of the electrode unit 810 is the same as that of the electrode unit 110 of the insulated electrode 100 of the first embodiment, to which reference may be made for related contents.

There is a plurality of temperature sensors 814 respectively accommodated in perforations 8132 of the corresponding dielectric elements 813. Four of the twenty electrode units 810 located in the middle two columns of the middle two rows are central electrode units 810B, and the other sixteen are peripheral electrode units 810A. In the present embodiment, there are eight temperature sensors 814, which are selectively arranged on the peripheral electrode units 810A. Specifically, they are respectively arranged on the eight electrode units 810 located in the third column of the first row, the fourth column of the first row, the third column of the last row, the fourth column of the last row, the second column of the second row, the fifth column of the second row, the second column of the third row, and the fifth column of the third row. The eight temperature sensors 814 are respectively arranged at the center of the main body part 8111 of the corresponding electrode unit 810.

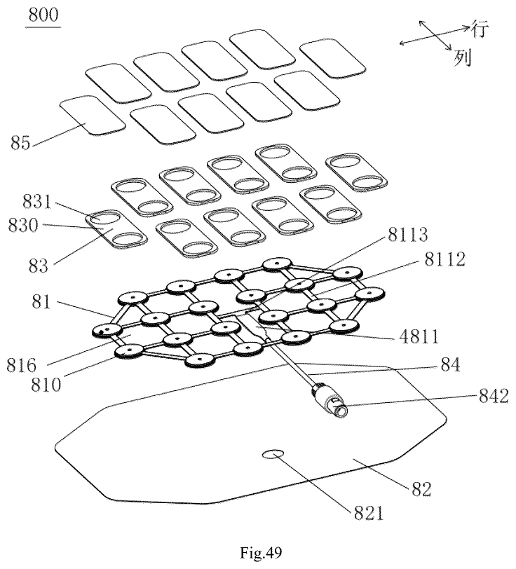

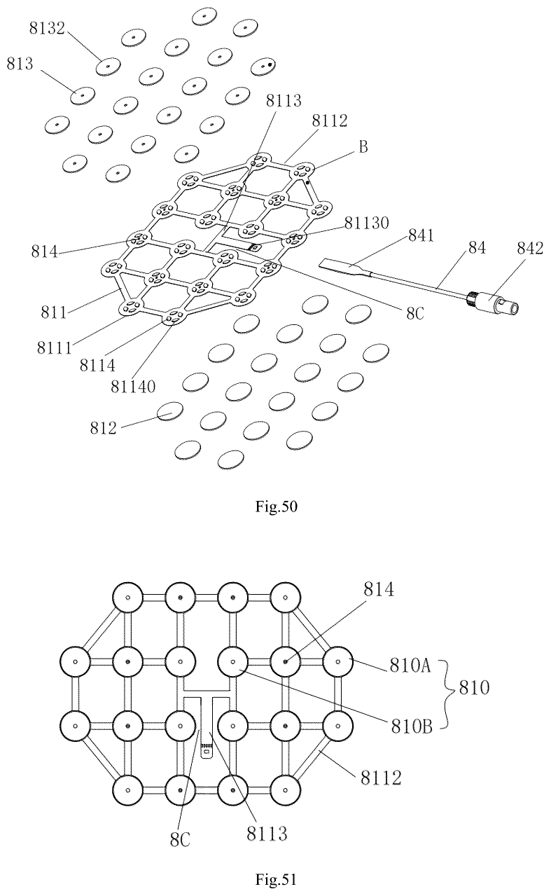

Referring to FIG. 49, the main body parts 8111 of the electrode units 810 arranged in four rows and six columns, the connecting parts 8112 connecting two adjacent electrode units 810 and the wiring part 8113 bridged between the two adjacent connecting parts 8112 together constitute the flexible circuit board 811 of the electrical functional component 81. The flexible circuit board 811 is in a grid shape. The dielectric elements 813 are arranged on the grid points of the flexible circuit board 811. It can be understood that the main body parts 8111 are the grid points of the flexible circuit board 811. In view of the formation of the electrode unit 810, the insulating plate 812 is arranged on the side of the main body part 8111 of the flexible circuit board 811 away from the human skin, and the dielectric element 813 is arranged on the side of the main body part 8111 of the flexible circuit board 811 facing the human skin. The temperature sensor 814 is selectively arranged on the side of the main body part 8111 of the flexible circuit board 811 facing the human skin. The arrangement of the main body parts 8111 of the flexible circuit board 811 is consistent with the arrangement of the electrode units 810.

The flexible circuit board 811 includes: an insulating substrate B and a plurality of conductive traces (not shown) embedded in the insulating substrate B. The conductive traces (not shown) embedded in the insulating substrates B of the main body parts 8111, the conductive traces (not shown) embedded in the insulating substrates B of the connecting parts 8112, and the conductive traces (not shown) embedded in the insulating substrate B of the wiring part 8113 are electrically connected. Conductive traces (not shown) are embedded in the insulating substrates B of some connecting parts 8112, and the remaining connecting parts 8112 only contain the insulating substrate B to strengthen the strength of the flexible circuit board 811. The conductive cores 81140 are exposed or protruded from the insulating substrate B of the main body part 8111. The insulating substrate B of flexible circuit board 811 may isolate the water vapor in the air around the insulated electrode 800 from the solder (not shown) located between the conductive cores 81140 of the conductive pad 8114 of the main body part 8111 of the flexible circuit board 811 and the dielectric element 813, and prevent the water vapor in the air away from the skin from eroding the solder (not shown) between the main body part 8111 and the dielectric element 813 of the flexible circuit board 811. The insulating substrate B of the flexible circuit board 811 and the insulating plates 812 function as a double isolation, which may prolong the service life of the insulated electrode 800. The golden fingers 81130 of the wiring part 8113 are exposed from the insulating substrate B.

The conductive traces (not shown) of the flexible circuit board 811 include: a path of the conductive trace (not shown) that connects all the conductive cores 81140 of the conductive pads 8115 located in respective main body parts 8111 in series, a path of the conductive trace (not shown) that connects the ground terminals (not shown) of respective temperature sensors 814 located on the main body parts 8111 in series, and a plurality of paths of the conductive traces (not shown) that respectively electrically connect the signal terminals (not shown) of respective temperature sensors 814 located on the corresponding main body parts 8111. These conductive traces (not shown) are electrically connected with the plurality of golden fingers 81130 of the wiring part 8113 in one-to-one correspondence.

The electrical functional component 81 is centrally adhered to the backing 82 through a biocompatible adhesive (not shown), and the backing 82 is provided with a threading hole 821 at a position corresponding to the end of the wiring part 8113. The threading hole 821 allows the end of the wire 84 to pass through and be electrically connected with the wiring part 8113, so as to avoid the wire 84 to be adhered between the backing 82 and the skin, and affect the tight adhesion of the insulated electrode 800 to the skin. It may further avoid air entering between the electrical functional component 81 and the human skin, which results in cryogenic burns due to the increase of heat generation of the electrical functional component 81 causing by the increase of the impedance between the electrical functional component 81 and the skin.

The support member 83 has a plurality of through holes 831 arranged therethrough, and the through holes 831 correspond to the electrode units 810. The support member 83 may be an integral sheet structure, and may improve the overall strength of the insulated electrode 800. A plurality of through holes 831 are arranged at intervals and are respectively arranged on the support member 83 around the corresponding electrode units 810. In the present embodiment, the support member 83 has a plurality of support units 830 with the same structure and independent to each other. The plurality of support units 830 are arranged at intervals. Each support unit 830 surrounds the periphery of the corresponding plurality of electrode units 810. Each support unit 830 has two through holes 831 arranged therethrough for accommodating two adjacent electrode units 810 in the same column, respectively. The support member 83 has ten support units 830. The thickness of the support member 83 is basically the same as the thickness of the electrode unit 810. After the support member 83 and the electrical functional component 81 are attached to the backing 82, the upper surface of the support member 83 is basically flush with the electrode unit 810. In other implementations, each support unit 830 may be provided with a single through hole 831 having a larger dimension, which surrounds the periphery of the plurality of electrode units 810 in columns.

The adhesive 85 is adhered to the support member 83 and the side of the electrode unit 810 away from the backing 82. The adhesive 85 has double-sided adhesiveness, which may keep the skin surface moist and relieve local pressure when contacting with the skin. The adhesive 85 is preferably made of conductive gel. The shape of the adhesive 85 is substantially the same as the shape of the support element 83. Since the support member 83 is flush with the upper surface of the electrode unit 810, the adhesive 85 smoothly covers the support member 83 and the electrode unit 810.

The insulated electrode 800 applies an alternating electric field to a patient's tumor site by the at least 10 electrode units 810 arranged thereon for tumor treatment. It may avoid the influence of insufficient electric treating fields on the therapeutic effect caused by the difference of tumor size, site and position, increase the coverage area of the electrode units 810 of the insulated electrode 800, enhance the electric field intensity applied to the tumor site for tumor treating fields therapy, increase the coverage range of the alternating electric field to the tumor site, and improve the therapeutic effect.

### An Alternate Embodiment of the Eighth Embodiment of the Insulated Electrode

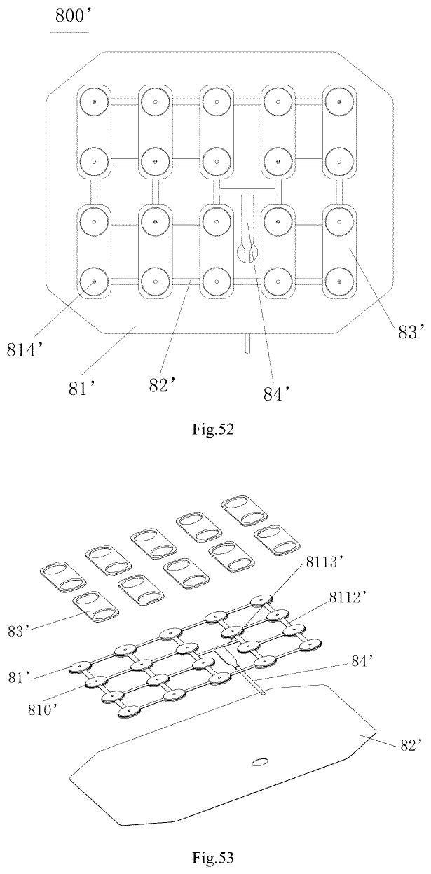

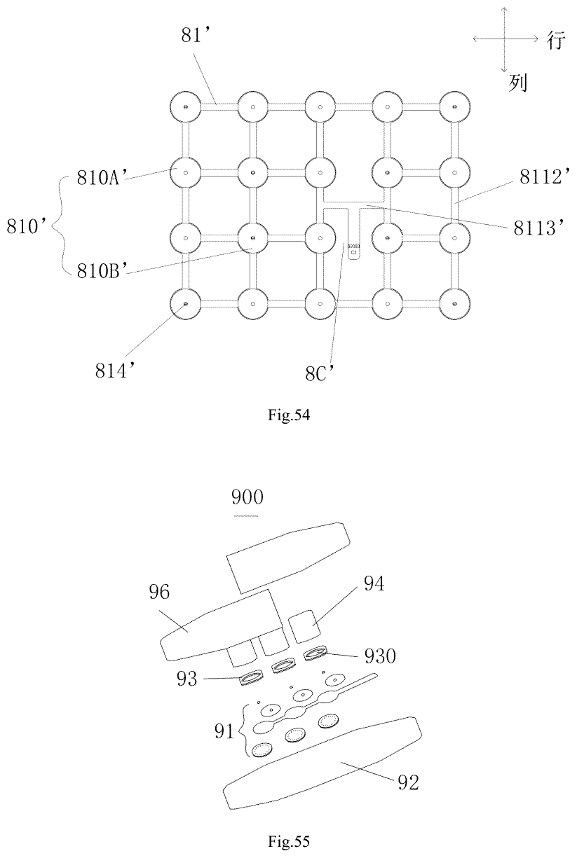

Referring to FIG. 52 to FIG. 54, the insulated electrode 800′ includes: a backing 82′; an electrical functional component 81′ adhered to the backing 82′; a support member 83′ adhered to the backing 82′; an adhesive (not shown) covering the support member 83′ and the corresponding parts of the electrical functional component 81′; and a wire 84′ electrically connected to the electrical functional component 81′.

The insulated electrode 800′ of the present embodiment is basically the same as the insulated electrode 800 of the eighth embodiment, and the only difference is that: the specific arrangement of the electrode units 810′ on the electrical functional component 81′ is different. Only the differences are described below, and other contents may be referred to the fourth embodiment.

The electrical functional component 81′ includes: a plurality of electrode units 810′ arranged in a rectangular array; a plurality of connecting parts 8112′ connecting two adjacent electrode units 810′; and a wiring part 8113′ electrically connected with a wire 84′. Each electrode unit 810′ is connected with at least two adjacent electrode units 810′ through connecting parts 8112′. Each electrode unit 810′ is connected with at least two connecting parts 8112′. A plurality of electrode units 810′ are arranged at intervals and distributed on the grid points of the electrical functional components 81′. A plurality of electrode units 810′ are distributed in an area surrounded by an array of at least three rows and four columns, and there are at least twelve electrode units and at most thirty electrode units, so that it may increase the coverage area of the electrode units 810′ of the insulated electrode 800′, enhance the electric field intensity applied to the tumor site for tumor treating fields therapy, increase the coverage range of the alternating electric field to the tumor site, and improve the therapeutic effect. The plurality of electrode units 810′ are distributed in an array area of three rows and four columns, and the number of the electrode units 810′ is twelve; or the plurality of electrode units 810′ are distributed in an array area of three rows and five columns, and the number of the electrode units 810′ is at least twelve and at most fifteen; or the plurality of electrode units 810′ are distributed in an array area of four rows and four columns, and the number of the electrode units 810′ is at least twelve and at most sixteen; or the plurality of electrode units 810′ are distributed in an array area of four rows and five columns, and the number of the electrode units 810′ is at least twelve and at most twenty; or the plurality of electrode units 810′ are distributed in an array area of four rows and six columns, and the number of the electrode units 810′ is at least twelve and at most twenty-four; or the plurality of electrode units 810′ are distributed in an array area of five rows and five columns, and the number of the electrode units 810′ is at least twelve and at most twenty-five; or the plurality of electrode units 810′ are distributed in an array area of five rows and six columns, and the number of the electrode units 810′ is at least twelve and at most thirty.

The electrode units 810′ in each row have the same number and are arranged in column-aligned manner. The electrode units 810′ in each column have the same number and are arranged in a row-aligned manner. The spacings between two adjacent electrode units 810′ arranged in rows are equal, and the spacings between two adjacent electrode units 810′ arranged in columns are also equal. Two adjacent electrode units 810′ in the same row are arranged in adjacent columns, and two adjacent electrode units 810′ in the same column are arranged in adjacent rows. The connecting part 8112′ is located between two adjacent electrode units 810′ in the same row or the same column. A plurality of connecting parts 8112′ between two adjacent electrode units 810′ arranged in rows have the same length. A plurality of connecting parts 8112′ between two adjacent electrode units 810′ arranged in columns have the same length. The spacing between two adjacent electrode units 810′ arranged in rows is different from the spacing between two adjacent electrode units 810′ arranged in columns. That is, the length of the connecting part 8112′ between two adjacent electrode units 810′ arranged in rows is different from the length of the connecting part 8112′ between two adjacent electrode units 810′ arranged in columns. Alternatively, the spacing between two adjacent electrode units 810′ arranged in rows is the same as the spacing between two adjacent electrode units 810′ arranged in columns. That is, the length of the connecting part 8112′ between two adjacent electrode units 810′ arranged in rows is the same as the length of the connecting part 8112′ between two adjacent electrode units 810′ arranged in columns.

At least two adjacent electrode units 810′ among the plurality of electrode units 810′ are arranged in a disconnected state. A spacing 8C′ is formed between the two adjacent electrode units 810′ arranged in the disconnected state, and the wiring part 8113′ passes through the spacing 8C′. The connecting part 8113′ may be in a shape of the Chinese character “-”, and laterally extended from a connecting part 8112′ corresponding to the spacing 8C′; or may be in a “T” shape and bridged between two connecting parts 8112′ respectively connected with two electrode units 810′ arranged in a disconnected state. The electrode units 810′ arranged in the disconnected state are located in an inner layer of the array area where the electrode unit 810′ is located. Every two of the electrode units 810′ of the electrical functional component 81′ located on the periphery are all connected by the connecting parts 8112′. That is, every two adjacent electrode units 810′ located at the periphery of the electrical functional component 81′ are connected by the connecting parts 8112′. Among the plurality of electrode units 810′, at least two adjacent electrode units 810′ located in adjacent rows and adjacent columns and diagonally arranged are arranged in a disconnected state. The wiring part 8113′ is located between the plurality of electrode units 810′, which may avoid the increase in manufacturing cost caused by the oversized overall size of the electrical functional component 81′.

In view of the distribution positions of the electrode units 810′ in the array, the plurality of electrode units 810′ may be divided into: a plurality of peripheral electrode units 810A′ at the periphery; and a plurality of central electrode units 810B′ surrounded by the peripheral electrode units 810A′. There are at least ten peripheral electrode units 810A′ and at least two central electrode units 810B′. Every two of all the peripheral electrode units 810A′ are connected by connecting parts 8112′. That is, the connecting parts 8112′ are arranged between all the two adjacent peripheral electrode units 810A′. Among the plurality of central electrode units 810B′, at least one central electrode units 810B′ and one of its adjacent peripheral electrode unit 810A′ or central electrode unit 810B′ located in the same row or in the same column are arranged in the disconnected state, to form a spacing 8C′ therebetween for the wiring part 8113′ to pass through.

The wiring part 8113′ may be laterally extended from the connecting part 8112′ opposite to the spacing 8C′ in a direction toward the spacing 8C′, and is substantially in a shape of the Chinese character “-”. The wiring part 8113′ and the connecting part 8112′ from which the wiring part 8113′ is laterally extended are vertically arranged, and are substantially in a “T” shape. The connecting part 8112′ from which the wiring part 8113′ is laterally extended is located between two adjacent peripheral electrode units 810A′, or may be located between a peripheral electrode unit 810A′ and a central electrode unit 810B′ adjacent to the peripheral electrode unit 810A′, or may further be located between two adjacent central electrode units 810B′. That is, the connecting part 8112′ from which the wiring part 8113′ is laterally extended connects two adjacent peripheral electrode units 810A′, or connects two adjacent central electrode units 810B′ or connects a peripheral electrode unit 810A′ and a central electrode unit 810B′ adjacent to the peripheral electrode unit 810A′. The wiring part 8113′ may also be in a “T” shape, and bridged between two connecting parts 8112′ respectively connected with two central electrode units 810B′ arranged in a disconnected state, or bridged between two connecting parts 8112′ respectively connected with a central electrode unit 810B′ and a peripheral electrode unit 810A′ adjacent to the central electrode unit 810B′ arranged in a disconnected state.

In other implementations, among the plurality of peripheral electrode units 810A′, at least two adjacent peripheral electrode units 810A′ are arranged in a disconnected state, and at least one of the peripheral electrode units 810A′ arranged in the disconnected state is connected with, by the connecting part 8112′, a central electrode unit 810B′ located in the adjacent row and the adjacent column and diagonally arranged. That is, some of the two adjacent peripheral electrode units 810′ are connected by the connecting part 8112′, and some of the two adjacent peripheral electrode units 810A′ are disconnected with no connecting part 8112′ arranged therebetween. The connecting part 8112′ is arranged between a peripheral electrode unit 810A′ and a central electrode unit 810B′ located in the adjacent row and the adjacent column of the peripheral electrode unit 810A′ and diagonally arranged, or between two adjacent peripheral electrode units 810A′, or between two adjacent central electrode units 810B′, or between a peripheral electrode unit 810A′ and its adjacent central electrode unit 810B′ located in the same row or the same column.

In the present implementation, a plurality of electrode units 810′ of the electrical functional component 81′ are arranged in four rows and five columns. The number of the electrode units 810′ of the electrical functional component 81′ is twenty. The number of the electrode units 810′ in each row is the same, and the number of the electrode units 810′ in each column is also the same. The number of the electrode units 810′ in each row is five. The number of the electrode units 810′ in each column is four. The electrode unit 810′ located in the third column of the second row is disconnected with the electrode unit 810′ located in the fourth column of the second row, and a spacing 8C′ is formed therebetween. The electrode unit 810′ located in the third column of the third row is disconnected with the electrode unit 810′ located in the fourth column of the third row, and a spacing 8C′ is also formed therebetween. The wiring part 8113′ is in a “T” shape, and is bridged between the connecting part 8112′ in the middle of the third column and the connecting part 8112′ in the middle of the fourth column. The connecting part 8112′ in the middle of the third column is arranged between the two electrode units 810′ respectively located in the second row of the third column and in the fourth row of the third column. The connecting part 8112′ in the middle of the fourth column is arranged between the two electrode units 810′ respectively located in the second row of the fourth column and in the third row of the fourth column. The connecting parts 8112′ are located between all the two adjacent electrode units 810′ in the same row or the same column except the two electrode units 810′ in the third column of the second row and the fourth column of the second row and the two electrode units 810′ in the third column of the third row and the fourth column of the third row. There are eight temperature sensors 814′, which are selectively arranged on the peripheral electrode units 810′.

In the present embodiment, the insulated electrode 800′ applies an alternating electric field to a patient's tumor site by a plurality of electrode units 810′ arranged thereon for tumor treatment. It may prevent the therapeutic effect from being affected by the insufficient intensity of the alternating electric fields applied to the tumor site for treating fields due to the difference of tumor size, site and position, increase the coverage area of the electrode units 810′ of the insulated electrode 800′, enhance the electric field intensity applied to the tumor site for tumor treating fields treatment, increase the coverage range of the alternating electric field to the tumor site, and improve the therapeutic effect.

### A Ninth Embodiment of the Insulated Electrode

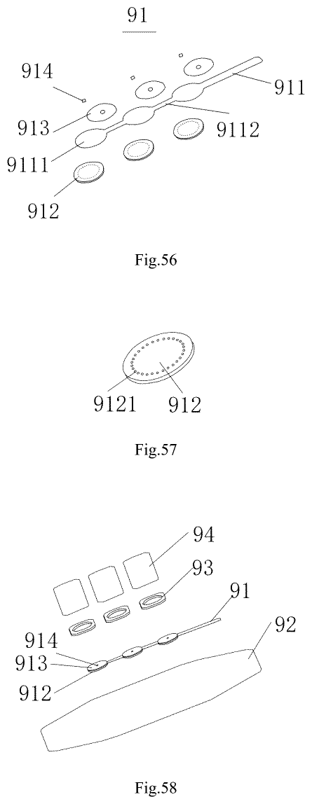

Referring to FIG. 55 to FIG. 58, the insulated electrode 900 of the present embodiment includes: an electrical functional component 91, a flexible backing 92, several support members 93, several adhesives 94, and a release paper 96 located on the adhesives 94 and able to adhere to the backing 92. The electrical functional components 91 include: a flexible circuit board 911, heat dissipation reinforcements 912 and dielectric components 913 respectively located on opposite sides of the flexible circuit board 911, and temperature sensors 914. The flexible circuit board 911 has several main body parts 9111 and several connecting parts 9112 connected to the main body parts 9111. The specific structure of the electrical functional component 91 is basically the same as the electrical functional components in the previous embodiments, and the only difference is that: the heat dissipation reinforcements 912 are used to replace the insulating plate in the previous embodiments to improve heat dissipation performance. The following only explains the heat dissipation reinforcements 912, and other structures of electrical functional component 91 will not be repeated.

The heat dissipation reinforcement 912 is substantially in a circular sheet shape, and has one side close to the patient's body surface and attached to the main body part 9111 of the flexible circuit board 911 through glue (not shown) and the other side away from the patient's body surface and attached to the backing 92 through biocompatible adhesive set on the backing 92. The heat dissipation reinforcements 912 are located on the side of the main body part 9111 of the flexible circuit board 911 away from the patient's body surface, so as to support the main body parts 9111 of the flexible circuit board 911, and to facilitate the welding of the temperature sensors 914 and the dielectric elements 913 to the main body parts 9111 of the flexible circuit board 911, respectively. The heat dissipation reinforcements 912 are sandwiched between the main body parts 9111 of the flexible circuit board 911 and the backing 92 after the electrical functional component 91 is assembled on the backing 92. The diameter of the heat dissipation reinforcement 912 is substantially the same as the diameter of the main body part 9111 of the flexible circuit board 911. The number of the heat dissipation reinforcements 912 is consistent with the number of the main body parts 9111 of the flexible circuit board 911. The number of heat dissipation reinforcements 912 is consistent with the number of the dielectric components 913. The heat dissipation reinforcements 912 are insulated from the conductive pads (not shown) on the main body parts 9111 of the flexible circuit board 911. The heat dissipation reinforcements 912 are electrically insulated from the flexible circuit board 911.

The heat dissipation reinforcements 912 are made of a material with a thermal conductivity coefficient greater than 200 W/mK, which may rapidly dissipate the heat generated by the dielectric elements 913 and the conductive pads (not shown) of the main body parts 9111 of the flexible circuit board 911 and the heat accumulated on the patient's body surface due to the long-term application of an alternating electric field to the body surface of the patient's tumor site through the insulated electrode 900, where the conductive pads are exposed from a side of the main body parts 9111 close to a patient's body surface, and are used for applying an alternating electric field. It improves the heat dissipation performance of the insulated electrode 900 during the process of the tumor treating fields therapy, and thereby ensuring that the treatment time is prolonged under a condition of keeping the magnitude of the alternating electric field applied to the insulated electrode 900 unchanged, and rendering tumor treating fields a good therapeutic effect.

The heat dissipation reinforcement 912 may be a metal plate, a metal alloy plate, or a graphene composite plate. The heat dissipation reinforcement 912 is further disposed with one or more heat dissipation holes 9121, which may further quickly dissipate heat through the backing 92. The heat dissipation holes 9121 are evenly distributed on the heat dissipation reinforcement 912. The heat dissipation holes 9121 are in a circular shape. Preferably, the heat dissipation reinforcement 912 is a metal plate or metal alloy plate with a thickness of 0.1 mm to 0.7 mm. Specifically, the heat dissipation reinforcement 912 is an aluminum plate or aluminum alloy plate with a thickness of 0.3 mm to 0.6 mm. Specifically, the heat dissipation reinforcement 912 is an aluminum plate with a thickness of 0.6 mm. There are thirty heat dissipation holes 9121 evenly distributed on the aluminum plate, and a diameter of the heat dissipation holes 9121 is 0.5 mm. Alternatively, the heat dissipation reinforcement 912 is a Type 6063 aluminum alloy plate with a thickness of 0.3 mm. The thermal conductivity coefficient of Type 6063 aluminum alloy is 201 W/mK. There are fifty heat dissipation holes 9121 evenly distributed on the Type 6063 aluminum alloy plate, and a diameter of the heat dissipation holes 9121 is 0.4 mm. Alternatively, the heat dissipation reinforcement 912 is made of graphene composite material with a thickness of 0.1 mm. The heat dissipation reinforcement 912 has a thermal conductivity coefficient greater than 300 W/mK.

The backing 92 is in a sheet shape, which is mainly made of materials that are flexible, permeable and insulating. The backing 92 has numeral permeable holes (not shown) arranged therethrough, which, when the backing 92 is adhered to a patient's body surface, allow the hair follicles and sweat glands of the skin of the patient's body surface covered by the backing 92 to breathe freely, preventing the sweat glands and hair follicles of the patient's body surface covered by the backing 92 from blockage, which otherwise leads to damage to the superficial layer of the patient's skin and causes skin inflammation. The backing 92 is a mesh fabric. Specifically, the backing 92 is a mesh non-woven fabric. The side of the backing 92 facing the patient's body surface is also coated with biocompatible adhesive, which is used to tightly adhere the backing 92 to the body surface of the patient's target area.

The support member 93 is substantially in a hollow ring shape, and has a through hole 930 arranged therethrough for the dielectric element 913 to pass through. The thickness of the support member 93 is approximately the same as the thickness of the dielectric element 913. The plane of the top end of the support member 93 is at the same vertical height as the surface of a side of the dielectric element 913 facing the patient's body surface. That is, the surface of the side of the support member 93 close to the patient's body surface is coplanar with the surface of the side of the dielectric element 913 close to the patient's body surface. The through hole 930 is in a circular shape, and has a diameter approximately the same as the diameter of the dielectric element 913. The through hole 930 is used for accommodating the dielectric element 913 after the insulated electrode 900 is assembled.

Both the support members 93 and the dielectric elements 913 are located on the same side of the flexible circuit board 911. The support members 93 and the heat dissipation reinforcements 912 are located on opposite sides of the flexible circuit board 911, respectively. The support member 93 is in a sheet shape, and may be made of polyethylene (PE), or PET, or thermal conductive silica gel sheet, or a soft, chemically stable, light weight, non-deformable and non-toxic insulating material composed of polyurethane, polyethylene, dispersant, flame retardant and carbon fiber. The support member 93 is arranged around the dielectric element 913 to position and support the adhesive 94, and at the same time, it may improve the wearing comfort of the insulated electrode 900. The flexible circuit board 911 is sandwiched between the support members 93 and the heat dissipation reinforcements 912. In the present embodiment, the support member 93 may be made of flexible foam. The side of the support member 93 close to the patient's body surface is attached to the adhesive 94, and the side of the support member 93 away from the patient's body surface is attached to the backing 92 through a biocompatible adhesive provided on the backing 92.

The adhesive 94 is in a sheet shape. One side of the adhesive 94 is attached to the side of the support member 93 and the dielectric element 913 close to the patient's body surface, and the other side of the adhesive 94 is adhered to the corresponding part of the release paper 96 when the insulated electrode 900 is not in use, and is adhered to the patient's body surface when the insulated electrode 900 is in use, so as to tightly adhere the insulated electrode 900 to the body surface corresponding to patient's tumor. The number of the adhesives 94 is the same as the number of the supporting members 93.

The release paper 96 is adhered to other parts of the backing 92 except the part adhered with the electrical functional component 91 through the biocompatible adhesive coated on the backing 92, and is used to cover the electrical functional component 91, the support members 93 arranged on the electrical functional component 91 and the adhesives 94, thereby protecting the adhesives 94 and the biocompatible adhesive on the backing 92 and preventing the adhesives 94 and the biocompatible adhesive on the backing 92 from being contaminated. The release paper 96 is made of an insulating material.

The temperature rising speeds of a patient's skin surface are tested for the insulated electrode 900 using metal plates or a metal alloy plates with a thermal conductivity coefficient larger than 200 W/mK (e.g., aluminum metal plates with a thermal conductivity coefficient of 237 W/mK) as the heat dissipation reinforcements 912 and an electrode using epoxy glass cloth laminate plates having the same area and thickness as the heat dissipation reinforcements 912 and with a thermal conductivity coefficient of 0.2 W/mK under conditions of the same applied electric field, the same electrode application position and the same treatment time. The results show that: the temperature rising speed of the patient's skin surface with the electrode using epoxy glass cloth laminate plates is about 0.0223° C./s (where the temperature test range is 36.5° C. to 39° C.), while the temperature rising speed of the patient's skin surface with the insulated electrode 900 of the present application is about 0.0178° C./s (where the temperature test range is 36.5° C. to 39° C.). The temperature rising speed of the electrode using aluminum metal plates as the heat dissipation reinforcements 912 is about 20.2% lower than that of the electrode using epoxy glass cloth laminate plates in actual use.

As verified by the above tests, by using heat dissipation reinforcements 912 with a thermal conductivity coefficient greater than 200 W/mK and provided with uniformly distributed heat dissipation holes 9121 thereon, the insulated electrode 900 has a high thermal conductivity and may quickly conduct out the heat accumulated on the skin of the patient's body surface when applying a relative high-tensity alternating electric field for a long time causes the surface temperature of the patient's tumor site to rise to a certain threshold, which result in a good therapeutic effect of the tumor treating fields therapy. And, it is not necessary to reduce the electric field intensity by rapidly reducing the alternating voltage applied to the electrode, so as to reduce the body surface temperature of the patient's tumor site, which may reduce the effect of the tumor treating fields.

### An Alternate Embodiment of the Ninth Embodiment of the Insulated Electrode

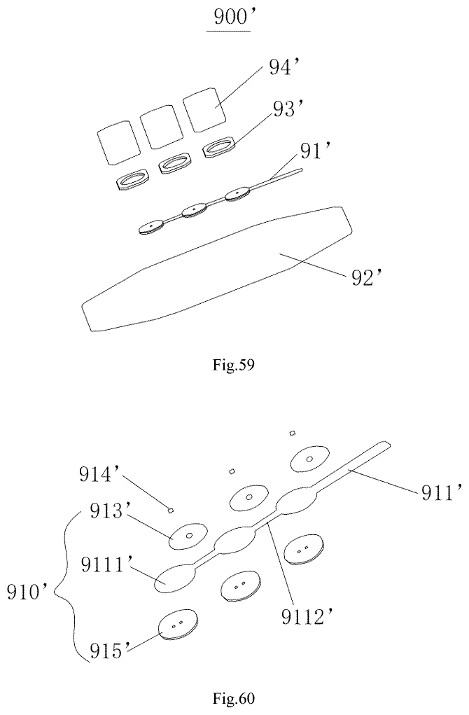

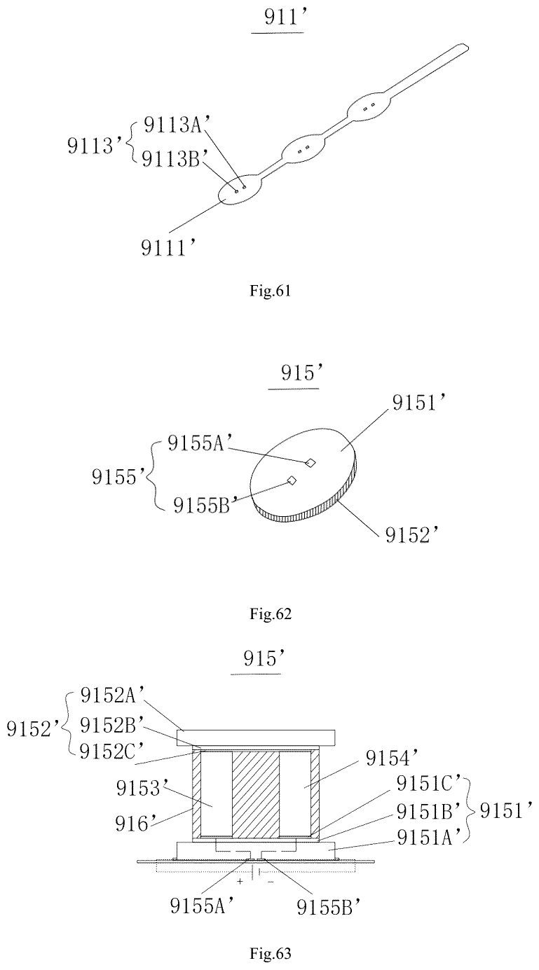

Referring to FIG. 59 to FIG. 63, the present embodiment is an active heat-absorbing insulated electrode 900′, which includes: an electrical functional component 91′, a backing 92′, support members 93′ and adhesives 94′. The electrical functional component 91′ includes a flexible circuit board 911′, dielectric elements 913′, temperature sensors 914′ and semiconductor refrigerators 915′. The flexible circuit board 911′ is provided with several main body parts 9111′ and several connecting parts 9112′ connecting the main body parts 9111′. The semiconductor refrigerators 915′ and the dielectric elements 913′ are respectively arranged on the opposite sides of the main body parts 9111′, and a dielectric element 913′, a main body part 9111′ and a semiconductor refrigerator 915′ constitute an electrode unit 910′ of the electrical functional component 91′. The specific structure of the insulated electrode 900′ is basically the same as that of the insulated electrode 900 in the ninth embodiment, and the only difference is that: the semiconductor refrigerators 915′ are used to replace the heat dissipation reinforcements 912, which may further improve the heat dissipation performance. The following description is only for the semiconductor refrigerators 915′, and other structures of the insulated electrode 900′ will not be repeated.

The side of the main body part 9111′ away from a patient's body surface has a welding part 9113′ exposed on the surface thereof. The welding part 9113′ includes: a first welding part 9113A′ and a second welding part 9113B′ arranged at intervals. The semiconductor refrigerator 915′ is welded with the welding part 9113′ to realize the electrical connection between the flexible circuit board 911′ and the semiconductor refrigerator 915′. The semiconductor refrigerator 915′ may quickly dissipate the heat accumulated on the skin of the patient's body 30 surface, so as to avoid cryogenic burns of the body surface of the patient's tumor site caused by the heat accumulation on the skin of the body surface corresponding to the patient's tumor site due to the long-term and continuous application of alternating electrical signal to the flexible circuit board 911′ and the dielectric element 913′ that are electrically connected.

The semiconductor refrigerator 915′ is in a circular sheet shape, and is welded with the welding part 9113′ of the main body part 9111′ of the flexible circuit board 911′ to realize the electrical connection with the flexible circuit board 911′. The semiconductor refrigerator 915′ is sandwiched between the main body part 9111′ and the backing 92′ of the flexible circuit board 911′, which may quickly dissipate the heat on the skin of the body surface of a patient to where the insulated electrode 900′ is adhered. One side of the semiconductor refrigerator 915′ is arranged on the main body part 9111′ of the flexible circuit board 911′ by welding, and the other side of the semiconductor refrigerator 915′ is attached to the backing 92′ by a biocompatible adhesive arranged on the backing 92′. The semiconductor refrigerator 915′ has a cooling end 9151′ close to the main body part 9111′ of the flexible circuit board 911′, a heat dissipation end 9152′ away from the main body part 9111′ of the flexible circuit board 911′, and an N-type semiconductor 9153′ and a P-type semiconductor 9154′ both sandwiched between the cooling end 9151′ and the heat dissipation end 9152′. The semiconductor refrigerator 915′ is attached to the backing 92′ through the heat dissipation end 9152′. The N-type semiconductor 9153′ and the P-type semiconductor 9154′ are all made by a special treatment of bismuth telluride with impurities. The semiconductor refrigerator 915′ realizes the electrical conduction between the cooling end 9151′ and the heat dissipation end 9152′ through the N-type semiconductor 9153′ and the P-type semiconductor 9154′.

The cooling end 9151′ has a welding pad 9155′ corresponding to the welding part 9113′ on the main body part 9111′ of the flexible circuit board 911′. The welding pad 9155′ includes: a positive electrode welding pad 9155A′ welded to the first welding part 9113A′ of the main body part 9111′ of the flexible circuit board 911′, and a negative electrode welding pad 9115B′ welded to the second welding part 9113B′ of the main body part 9111′ of the flexible circuit board 911′. The cooling end 9151′ is arranged on the flexible circuit board 911′ through the welding pad 9155′, and is electrically connected with the flexible circuit board 911′ through the welding pad 9155′. The cooling end 9151′ includes: a cold-end ceramic sheet 9151A′ welded to the main body part 9111′ of the flexible circuit board 911′; a cold-end heat conductor 9151B′ arranged on the side of the cold-end ceramic sheet 9151A′ away from the flexible circuit board 911′; and two cold-end metal conductors 9151C′ arranged on the cold-end heat conductor 9151B′. The two cold-end metal conductors 9151C′ are arranged at intervals in parallel, and are respectively connected with the N-type semiconductor 9153′ and the P-type semiconductor 9154′.

The welding pad 9155′ is arranged on the side of the cold-end ceramic sheet 9151A′ facing the flexible circuit board 911′. The cold-end ceramic sheet 9151A′ is substantially in a circular sheet shape, and has a dimension slightly smaller than the dimension of the main body part 9111′ of the flexible circuit board 911′. There is a spacing (not shown) between the cold-end ceramic sheet 9151A′ and the main body part 9111′ of the flexible circuit board 911′ after the cold-end ceramic sheet 9151A′ being welded with the welding pad 9155′ through the welding part 9113′. The spacing (not shown) is filled with a sealant 916′, which can prevent the water vapor of a patient's body surface from entering the spacing (not shown) between the cold-end ceramic sheet 9151A′ and the main body part 9111′ of the flexible circuit board 911′ to erode the welding position and cause short circuit, which will affect the electrical connection between the cold-end ceramic sheet 9151A′ and the flexible circuit board 911′. The cold-end ceramic sheet 9151A′ is sandwiched between the main body part 9111′ of the flexible circuit board 911′ and the cold-end heat conductor 9151B′.

The cold-end heat conductor 9151B′ is in a form of an integrated circular sheet structure, and is used for fixing the cold-end metal conductors 9151C′ to the cold-end ceramic sheet 9151A′. The dimension of the cold-end heat conductor 9151B′ is slightly smaller than the dimension of the cold-end ceramic sheet 9151A′. The cold-end heat conductor 9151B′ and the main body part 9111′ of the flexible circuit board 911′ are respectively located on the opposite sides of the cold-end ceramic sheet 9151A′. The cold-end heat conductor 9151B′ is made of a heat conducting and non-conductive material. The cold-end heat conductor 9151B′ may be made of a thermal conductive silica gel. The side of the cold-end heat conductor 9151B′ away from the cold-end ceramic sheet 9151A′ is provided with two concave spaces (not labeled) that are respectively recessed downward from the top end, and are used for accommodating the cold-end metal conductors 9151C′. The two concave spaces (not labeled) are arranged at intervals and are substantially in the circular shape.

The two cold-end metal conductors 9151C′ are respectively arranged in the corresponding concave spaces (not labeled) and protrude from the top end of the cold-end heat 30 conductor 9151B′. The parts of the two cold-end metal conductors 9151C′ protruding from the cold-end heat conductor 9151B′ have the same height. That is, the sides of the two cold-end metal conductors 9151C′ protruding from the cold-end heat conductor 9151B′ are at the same level. The dimensions of the two cold-end metal conductors 9151C′ arranged at intervals are completely the same. The cold-end metal conductor 9151C′ is substantially in a circular sheet shape and is made of the same material as the N-type semiconductor 9153′ and the P-type semiconductor 9154′. The cold-end metal conductor 9151C′ is preferably made of copper. The diameter of the cold-end metal conductor 9151C′ is approximately the same as the diameter of the concave space (not labeled) of the cold-end heat conductor 9151B′. The two cold-end metal conductors 9151C′ arranged at intervals are arranged on the side of the cold-end heat conductors 9151B′ away from the flexible circuit board 911′. The cold-end heat conductor 9151B′ is sandwiched between the cold-end metal conductors 9151C′ and the cold-end ceramic sheet 9151A′. Two corresponding conductive traces (not shown) are respectively arranged in the cold-end ceramic sheet 9151A′ and the cold-end heat conductor 9151B′. The ends of the two conductive traces on the cold-end ceramic sheet 9151A′ are connected with the welding pads 9155A′ and 9155B′, respectively. The two cold-end metal conductors 9151C′ arranged at intervals are assembled on the cold-end ceramic sheet 9151A′ through the cold-end heat conductor 9151B′.

Both the N-type semiconductor 9153′ and the P-type semiconductor 9154′ are substantially in a cylindrical shape. The N-type semiconductor 9153′ and the P-type semiconductor 9154′ are assembled on the corresponding cold-end metal conductors 9151C′, respectively. The diameter of the N-type semiconductor 9153′ is the same as that of the corresponding cold-end metal conductor 9151C′. The diameter of the P-type semiconductor 9154′ is the same as that of the corresponding cold-end metal conductor 9151C′. Both the N-type semiconductor 9153′ and the P-type semiconductor 9154′ are made of copper. The N-type semiconductor 9153′ and the P-type semiconductor 9154′ have the same thickness.

The heat dissipation end 9152′ includes: a hot-end ceramic sheet 9152A′ attached to the backing 92′ by a biocompatible adhesive provided on the backing 92′; a hot-end heat transfer member 9152B′ arranged on one side of the hot-end ceramic sheet 9152A′ away from the backing 92′; and a hot-end metal conductor 9152C′ arranged on the other side of the hot-end heat transfer member 9152B′. The hot-end metal conductor 9152C′ is placed on the N-type semiconductor 9153′ and the P-type semiconductor 9154′. The hot-end metal conductor 9152C′ is supported by the N-type semiconductor 9153′ and the P-type semiconductor 9154′. The N-type semiconductor 9153′ is sandwiched between the hot-end metal conductor 9152C′ and one cold-end metal conductor 9151C′. The P-type semiconductor 9154′ is sandwiched between the hot-end metal conductor 9152C′ and the other cold-end metal conductor 9151C′. That is, one end of the N-type semiconductor 9153′ abuts against the corresponding cold-end metal conductor 9151C′, and the other end of the N-type semiconductor 9153′ abuts against the corresponding part of the hot-end metal conductor 9152C′. One end of the P-type semiconductor 9154′ abuts against the other cold-end metal conductor 9151C′, and the other end of the P-type semiconductor 9154′ abuts against the corresponding part of the hot-end metal conductor 9152C′. The N-type semiconductor 9153′ and the P-type semiconductor 9154′ are connected by the hot-end metal conductor 9152C′.

The hot-end ceramic sheet 9152A′ is in a circular sheet shape, and has a dimension approximately the same as that of the cold-end ceramic sheet 9151A′. The hot-end ceramic sheet 9152A′ is sandwiched between the backing 92′ and the hot-end heat transfer member 9152B′. The hot-end heat transfer member 9152B′ is sandwiched between the hot-end ceramic sheet 9152A′ and the hot-end metal conductor 9152C′. The hot-end heat transfer member 9152B′ is in a circular sheet shape, and has a dimension slightly smaller than the dimension of the hot-end ceramic sheet 9152A′. The dimension of the hot-end heat transfer member 9152B′ is approximately the same as the dimension of the cold-end heat transfer member 9151B′. The hot-end heat transfer member 9152B′ assembles the hot-end metal conductor 9152C′ on the hot-end ceramic sheet 9152A′. The side of the hot-end heat transfer member 9152B′ away from the hot-end ceramic sheet 9152A′ is recessed from bottom to top and form an accommodating space (not labeled) for accommodating the hot-end metal conductor 9152C′. The hot-end heat transfer member 9152B′ is made of a heat transfer and non-conductive material. The hot-end heat transfer member 9152B′ may be made of a thermal conductive silica gel. The parts of the hot-end metal conductor 9152C′ that protrudes from the hot-end heat transfer member 9152B′ are respectively connected to ends of the N-type semiconductor 9153′ and the P-type semiconductor 9154′. The hot-end metal conductor 9152C′ and the cold-end metal conductor 9151C′ are made of the same material. Both the hot-end metal conductor 9152C′ and the cold-end metal conductor 9151C′ are made of copper.

The semiconductor refrigerator 915′ also seals the cold-end metal conductor 9151C′, the N-type semiconductor 9153′, the P-type semiconductor 9154′ and the hot-end metal conductor 9152C′ sandwiched between the hot-end heat transfer member 9152B′ of the heat dissipation end 9152′ and the cold-end heat conductor 9151B′ of the cooling end 9151′ through the sealant 916′, so as to avoid short circuits inside the semiconductor refrigerator 915′ and between the welding pad 9155′ of the semiconductor refrigerator 915′ and the welding part (not shown) of the flexible circuit board 911′ due to the water vapor generated during heat exchange of the cooling end 9151′ and the heat dissipation end 9152′ entering into the semiconductor refrigerator 915′ or entering between the semiconductor refrigerator 915′ and the flexible circuit board 911′. The semiconductor refrigerator 915′ realizes the electrical conduction between the positive electrode welding pad 9155A′ and the negative electrode welding pad 9155B′ of the cold-end ceramic sheet 9151A′ through two conductive traces (not labeled) which are respectively connected with the positive electrode welding pad 9155A′ and the negative electrode welding pad 9151B′ of the cold-end ceramic sheet 9151A′ being connected with the corresponding conductive trace (not labeled) of the cold-end heat conductor 9151B′, the conductive trace (not labeled) of the cold-end heat conductor 9151B′ being respectively in contact with the ends of the two cold-end metal conductors 9151C′ arranged on the cold-end heat conductor 9151B′, the other ends of the two cold-end metal conductors 9151C′ being respectively in contact with one end of the N-type semiconductor 9153′ and one end of the P-type semiconductor 9154′, the other end of the N-type semiconductor 9153′ and the other end of the P-type semiconductor 9154′ being both in contact with the hot-end metal conductor 9152C′ arranged on the hot-end heat transfer member 9152B′. By welding the positive electrode welding pad 9155A′ of the cold-end ceramic sheet 9151A′ with the welding part 9113A′ of the main body part 9111′ of the flexible circuit board 911′ and welding the negative electrode welding pad 9115B′ of the cold-end ceramic sheet 9151A′ with the welding part 9113B′ of the main body part 9111′ of the flexible circuit board 911′, the semiconductor refrigerator 915′ realizes the electrical connection with the flexible circuit board 911′, and thus it may receive control signals from the flexible circuit board 911′.

The semiconductor refrigerator 915′ is made by using the Peltier effect of semiconductors. When a large amount of heat is accumulated on the body surface of a patient's tumor site where the insulated electrode 900′ is applied, the tumor treating fields system 1000 containing the insulated electrode 900′ inputs direct current to the semiconductor refrigerator 915′ through the flexible circuit board 911′. The current in the internal loop of the semiconductor refrigerator 915′ flows from the positive electrode welding pad 9155A′ of the cold-end ceramic sheet 9151A′ and passes through the cold-end heat conductor 9151B′ to sequentially flow to the cold-end metal conductors 9151C′ electrically connected with the positive electrode welding pad 9155A′ of the cold-end ceramic sheet 9151A′, the N-type semiconductor 9153′, the hot-end metal conductor 9152C′, the P-type semiconductor 9154′, the cold-end metal conductor 9151C′ electrically connected with the P-type semiconductor 9154′, the conductive trace of the cold-end heat conductor 9151B′ electrically connected with the negative electrode welding pad 9155B′ of the cold-end ceramic sheet 9151A′, and to the negative electrode welding pad 9155B′ of the cold-end ceramic sheet 9151A′.

The electrons of the P-type semiconductor 9154′ of the semiconductor refrigerator 915′ sequentially pass through the cold-end metal conductor 9151C′ in contact with the P-type semiconductor 9154′, the conductive trace of the cold-end ceramic sheet 9151A′ and the cold-end heat conductor 9151B′, the cold-end metal conductor 9151C′ in contact with the N-type semiconductor 9153′, the N-type semiconductor 9153′, the hot-end metal conductor 9152C′, and to the P-type semiconductor 9154′. At the cooling end 9151′ of the semiconductor refrigerator 915′, when the electrons flow from the P-type semiconductor 9154′ to the N-type semiconductor 9153′ through the cold-end ceramic sheet 9151A′, the charge moves from a low-energy level position to a high-energy level position, which absorbs heat from the outside. At the heat dissipation end 9152′ of the semiconductor refrigerator 915′, when the electrons flow from the N-type semiconductor 9153′ to the P-type semiconductor 9154′ through the hot-end metal conductor 9152C′, the charge moves from a high-energy level position to a low-energy level position, which dissipates heat to outside. That is, when direct current is input to the semiconductor refrigerator 915′ by the flexible circuit board 911′, the temperature of the cooling end 9151′ of the semiconductor refrigerator 915′ decreases and will actively absorb heat from the flexible circuit board 911′ through the cold- end ceramic sheet 9151A′, while the temperature of the heat dissipation end 9152′ of the semiconductor refrigerator 915′ increases and will exchange heat with the air outside through the hot-end ceramic sheet 9152A′ to dissipate heat. The heat absorbed by the cold-end ceramic sheet 9151A′ is transferred to the outside of the insulated electrode 900′ through the cold-end heat conductor 9151B′, the cold-end metal conductor 9151C′, the N-type semiconductor 9153′ and the P-type semiconductor 9154′, the hot-end metal conductor 9152C′, the hot-end heat transfer member 9152B′ and the hot-end ceramic sheet 9152A′, so as to avoid cryogenic burns of the skin of a patient's body surface caused by heat accumulation on the skin of the patient's body surface where the insulated electrode 900′ is applied when conducting a long-term and continuous tumor treating fields therapy. There is no need to avoid cryogenic burns of the skin of the patient's body surface by stopping the treatment, so that the patient has a longer tumor treatment time and obtains better therapeutic effect.

The temperature rising speeds of a patient's skin surface are tested for the insulated electrode 900′ using the semiconductor refrigerator 915′ and an electrode using epoxy glass cloth laminate plates having the same dimension as the semiconductor refrigerator 915′ under conditions of the same applied electric field, the same electrode application position and the same treatment time. The results show that: the temperature rising speed of the patient's skin surface with the electrode using the epoxy glass cloth laminate plates is about 0.0223° C./s (where the temperature test range is 36.5° C. to 39° C.), while the temperature rising speed of the patient's skin surface with the insulated electrode 900′ of the present application is about 0.0108° C./s (where the temperature test range is 36.5° C. to 39° C.). The temperature rising speed of the electrode using the semiconductor refrigerator 915′ is about 51.5% lower than that of the electrode using epoxy glass cloth laminate plates in actual use.

As verified by the above tests, by arranging semiconductor refrigerators 915′ on a side of the main body part 9111′ of the flexible circuit board 911′ away from the patient's body surface, the insulated electrode 900′ may allow the cooling ends 9151′ of the semiconductor refrigerators 915′ to actively perform cooling when a controller of the tumor treating system 1000 inputs direct current to the semiconductor refrigerators 915′ through the flexible circuit board 911′, and absorb the heat generated by a long-term and continuous tumor treating fields therapy and located on a patient's body surface and transmitted to the flexible circuit board 911′ through the adhesives 94′ and the dielectric elements 913′. The heat is then transmitted to the outside of the electrode through the heat dissipation ends 9152′, so as to quickly dissipate the heat at the body surface of the patient's tumor site, and achieve the goal of cooling. At the same time, the patient may have a relatively long treatment time, ensuring a better therapeutic effect. It is not necessary to reduce the alternating voltage applied to the flexible circuit board 911′ or reduce the alternating voltage applied to the patient's tumor site through the dielectric elements 913′ to avoid cryogenic burns of the skin of the patient's body surface where the electrode is applied.

The backing 92′ may also be provided with an opening (not shown) at a position corresponding to the hot-end ceramic sheet 9152A′ of the semiconductor refrigerator 915′, so as to expose the hot-end ceramic sheet 9152A′ of the semiconductor refrigerator 915′ to the air, and further enhance the heat dissipation effect. According to temperature test data of the samples of different human sites corresponding to different indications, the semiconductor refrigerator 915′ may be welded only at the electrode units 910′ in areas with a higher temperature, which facilities to reduce the overall weight of the insulated electrode 900′.

The present application further provides a tumor treating fields system 1000, which includes: an insulated electrode 900′, and a controller (not shown) electrically connected with the insulated electrode 900′. The controller (not shown) detects the temperature of the adhesive 94′ which is in contact with the body surface of the patient's tumor site through the temperature sensor 914′ arranged on the flexible circuit board 911′ of the insulated electrode 900′, and then determines whether to input direct current to the semiconductor refrigerators 915′ through the flexible circuit board 911′.

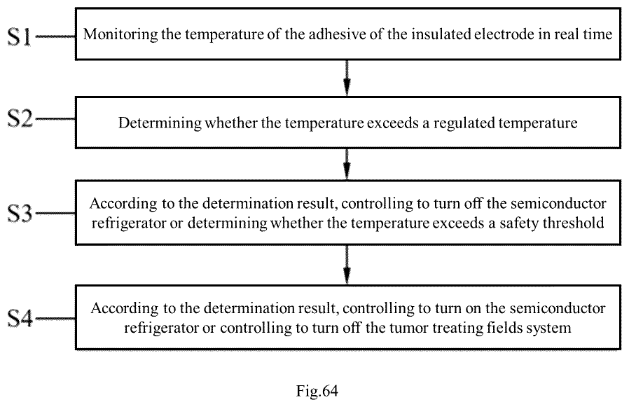

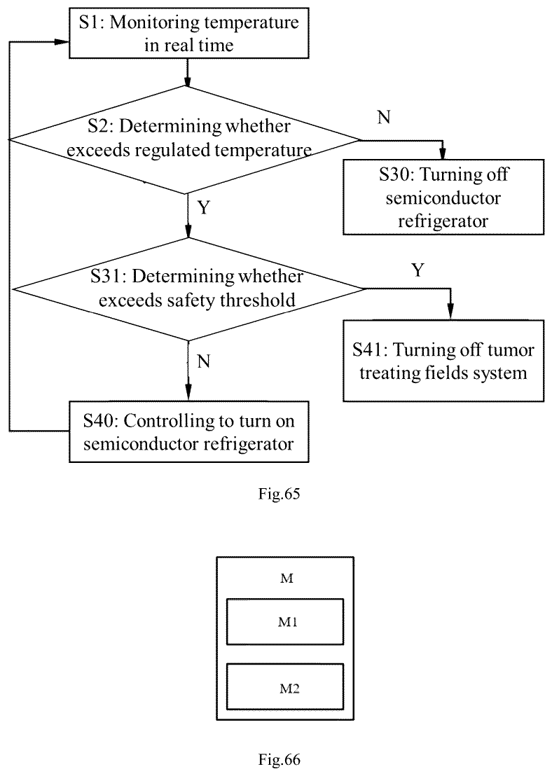

Referring to FIG. 64 and FIG. 65, the present application further provides a temperature control method of a tumor treating fields system using the above insulated electrode 900′. The tumor treating fields system includes the above insulated electrode 900′ and a controller (not shown) electrically connected with the insulated electrode 900′. The temperature control method specifically includes the following steps: step S1: monitoring the temperature of the adhesive 94′ of the insulated electrode 900′ in real time; step S2: determining whether the temperature obtained by monitoring exceeds a regulated temperature; step S3: according to the determination result, controlling to turn off the semiconductor refrigerator 915′, or determining whether the temperature obtained by monitoring exceeds a safety threshold; step S4: according to the determination result of whether the safety threshold is exceeded in step S3, controlling to turn on the semiconductor refrigerator 915′, or controlling to turn off the tumor treating fields system.

In step S1, aside of the adhesive 94′ of the insulated electrode 900′ adheres to the body surface skin of the patient's tumor site, and the other side is attached to the support member 93′ and the dielectric element 913′. In step S1, monitoring the temperature of the adhesive 94′ in real time is monitoring by the temperature sensor 914′ arranged on the flexible circuit board 911′. Detecting the temperature of the adhesive 94′ attached to the patient's tumor site through the temperature sensor 914′ is to obtain the temperature of the body surface skin of the patient's tumor site to which the adhesive 94′ is attached through the temperature sensor 914′.

In step S2, determining whether the temperature obtained by monitoring exceeds a regulated temperature is to compare the temperature obtained by monitoring with the regulated temperature.

In step S3, the determination result includes that: the temperature obtained by monitoring is lower than the regulated temperature, or the temperature obtained by monitoring is higher than the regulated temperature.

The step S3, according to the determination result, controlling to turn off the semiconductor refrigerator 915′, or determining whether the temperature obtained by monitoring exceeds a safety threshold, specifically includes the following steps: step S30: when the temperature obtained by monitoring is lower than the regulated temperature, turning off the semiconductor refrigerator 915′; step S31: when the temperature obtained by monitoring is higher than or equal to the regulated temperature, determining whether the temperature obtained by monitoring exceeds the safety threshold. In step S31, determining whether the temperature obtained by monitoring exceeds the safety threshold is obtained by comparing the temperature obtained by monitoring with the safety threshold. The difference between the safety threshold and the regulated temperature is within 4° C., so as to avoid discomfort caused by low temperature. Preferably, the regulated temperature is 39° C. and the safety threshold is 41° C.

The step S4, according to the determination result of whether the safety threshold is exceeded in step S3, includes: the monitored temperature exceeds or is equal to the safety threshold, or the monitored temperature is lower than the safety threshold. The step S4, according to the determination result of whether the safety threshold is exceeded in step S3, controlling to turn on the semiconductor refrigerator 915′, or controlling to turn off the tumor treating fields system, specifically includes the following steps: step S40: when the temperature obtained by monitoring is lower than the safety threshold, controlling to turn on the semiconductor refrigerator 915′ and repeat steps S1 to S3; step S41: when the temperature obtained by monitoring is higher than or equal to the safety threshold, controlling to turn off the tumor treating fields system.

In step S4, controlling to turn on the semiconductor refrigerator 915′ is specifically achieved by controlling the flexible circuit board 911′ to input direct current to the semiconductor refrigerator 915′. The step S40, controlling to turn on the semiconductor refrigerator 915′, refers to inputting direct current to the semiconductor refrigerator 915′ through the flexible circuit board 911′ to place the semiconductor refrigerator 915′ in an ON status. The step S41, controlling to turn off the tumor treating fields system, refers to place the tumor treating fields system in an OFF status, which is specifically achieved by turning off the power supply of the tumor treating fields system.

Controlling to place the semiconductor refrigerator 915′ in an ON status is achieved by inputting direct current to the semiconductor refrigerator 915′. Controlling to place the tumor treating fields system in an OFF status is achieved by turning off the direct current input to the semiconductor refrigerator 915′.

When the semiconductor refrigerator 915′ is in an ON status, the cooling end 9151′ of the semiconductor refrigerator 915′ may actively absorb the heat generated on the body surface of the patient's tumor site which is transmitted to the flexible circuit board 911′ through the adhesive 94′ and the dielectric element 913′, as well as the heat generated between the conductive pad (not shown) of the flexible circuit board 911′ and the dielectric element 913′ during the operation of the insulated electrode 900′, and the heat is quickly dissipated through the heat dissipation end 9152′ of the semiconductor refrigerator 915′, thereby quickly reducing the temperature of the adhesive 94′ of the insulated electrode 900′, and thereby lowering the body surface temperature of the patient's tumor site. It is not necessary to reduce the alternating current applied to the insulated electrode 900′ or reduce the alternating electric field applied to the dielectric element 913′ through the conductive pad (not shown) of the flexible circuit board 911′ to achieve the goal of reducing the body surface temperature of the patient's tumor site. It may achieve long-term and continuous tumor treating fields therapy, and improve the therapeutic effect.

Under normal circumstances, after the semiconductor refrigerator 915′ is turned on, the surface temperature of the skin will slowly decrease. However, due to some internal or external reasons, there is a small probability that the electric field may loss control. In order to avoid the temperature rising too fast due to excessive electric field, when the detected temperature exceeds the safe upper limit (e.g., 41° C.), the power supply of the tumor treating fields system will be turned off. The tumor treating fields system needs to be turned on manually to back to work.

With such settings, a temperature ladder (39° C., 41° C.) may be used for controlling, so as to keep the tumor treating fields system running as much as possible while reducing the surface temperature of the skin. Compared with the traditional method of cooling by directly turning off the tumor treating fields system, such a temperature control method may allow for more time to implement alternating electric field treatment and improve the therapeutic effect.

### A First Embodiment of the Treating Fields System 1000

Due to the application of an alternating electric field, the human body has its own impedance, resulting in an increase in heat on applied surface. The upper limit of the safe temperature of the human body surface is 41° C., and exceeding this upper limit may is likely to lead to cryogenic burns of the skin. In order to avoid cryogenic burns of the skin, it is necessary to monitor and control the temperature of the insulation electrodes on the applied surface (or human body surface) in real time. Referring to FIG. 66 to FIG. 72, the present application further provides a method of the tumor treating fields system 1000 applying alternating electrical signals.

FIG. 66 is a schematic block diagram of the electric field generator M of the tumor treating fields system 1000 of the present application. As shown in FIG. 66, the electric field generator M includes an alternating current signal generator M1 and a signal controller M2.

Referring to FIG. 1, the alternating current signal generator M1 is configured to generate at least two alternating current signals. The at least two alternating current signals are output to the corresponding at least two pairs of the insulated electrodes 1 and 2, so as to generate alternating electric fields 3 and 4 in at least two directions between the at least two pairs of the insulated electrodes 1 and 2.

The signal controller M2 is configured to obtain temperature information of the insulated electrodes 1 and 2 attached to the body surface of the tumor site, and individually control output of each of the at least two alternating current signals based on the temperature information, so as to selectively apply to the corresponding insulated electrodes 1 and 2 an alternating current signal that generates the corresponding alternating electric field of the alternating electric fields in the at least two directions.

In one example, the signal controller M2 controls whether each alternating current signal generated by the alternating current signal generator M1 to be output to the corresponding first pair of insulated electrodes 1 or the second pair of insulated electrodes 2. Each pair of insulated electrodes can be any one of the afore-mentioned insulated electrodes 100, 100′, 200, 300, 400, 400′, 500, 600, 600′, 700, 700′, 700″, 800, 800′, 900 and 900′. When the signal controller M2 controls the first alternating current signal to be output to the corresponding first pair of insulated electrodes 1, the alternating current signal will generate a first electric field 3 in a first direction between the two insulated electrodes 1. The two insulated electrodes 1 can be attached to the body surface of the subject, so that the first electric field 3 in the first direction can be applied to the attached site. Similarly, when the signal controller M2 controls the second alternating current signal, which is different from the first alternating current signal, generated by the alternating current signal generator M1 to be output to the corresponding second pair of insulated electrodes 2, the alternating current signal will generate a second electric field 4 in a second direction between the two insulated electrodes 2. Based on the temperature information of the body surface of the subject to which the first pair of insulated electrodes 1 is attached corresponding to the first alternating current signal, and the temperature information of the second pair of insulated electrodes 2 corresponding to the second alternating current signal, the signal controller M2 may independently control whether the first alternating current signal and the second alternating current signal to be output to the corresponding first pair of insulated electrodes 1 or the second pair of insulated electrodes 2.

To sum up, the electric field generator M may use the signal controller M2 to control the various outputs of the alternating current signal generator M1. Due to the separate control of the various alternating current signals, the controllability of applying electric fields to the corresponding insulated electrodes 1 and insulated electrodes 2 is improved.

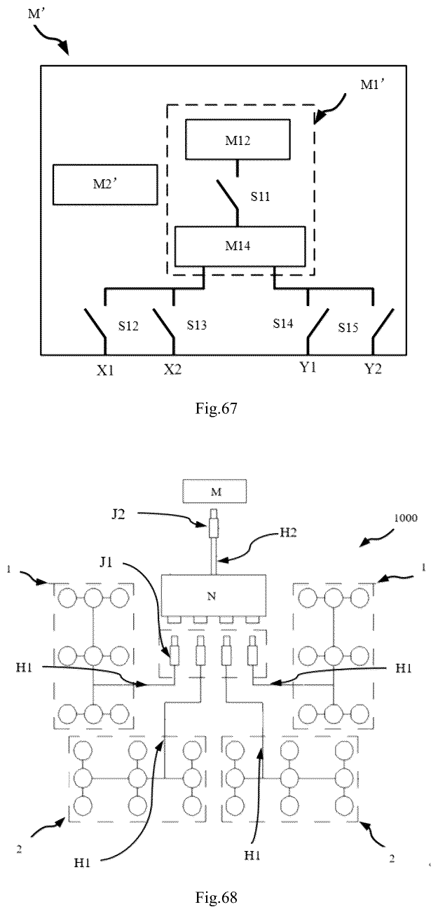

FIG. 67 is a schematic block diagram of another implementation of the electric field generator. As shown in FIG. 67, the alternating current signal generator M1′ of the electric field generator M′ may further include: a direct current signal source M12 and a signal converter M14. The direct current signal source M12 is configured to generate the direct current signal. In one example, a high-power direct current signal source may be used. The signal converter M14 is configured to convert the direct current signal into at least two alternating current signals.

The alternating current signal generator M1′ further includes a direct current signal switch S11. The direct current signal switch S11 is electrically connected between the direct current signal source M12 and the signal converter M14. The signal controller M2′ of the electric field generator M′ is configured to control a supply of the direct current signal from the direct current signal source M12 to the signal converter M14 by controlling the direct current signal switch S11.

The electric field generator M′ further includes at least two pairs of output terminals. FIG. 67 illustrates the two pairs of output terminals (X1, X2) and (Y1, Y2). Each pair of output terminals is configured for supplying corresponding alternating current signal of the at least two alternating current signals from the alternating current signal generator M1′. The signal converter M14 converts the direct current signal source M12 into two intermediate and high frequency alternating current signals. The two alternating current signals are respectively defined as: a X-direction alternating current signal transmitted along an X-direction loop; and a Y-direction alternating current signal transmitted along a Y-direction loop, in which the pair of output terminals (X1, X2) constitutes the X-direction loop, and the pair of output terminals (Y1, Y2) constitutes the Y-direction loop. The X-direction alternating current signal generates an X-direction electric field between the corresponding first pair of insulated electrodes 1, and the Y-direction alternating current signal generates a Y-direction electric field between the corresponding second pair of insulated electrodes 2.

The electric field generator M′ further includes at least two pairs of switches S12, S13, S14 and S15. The at least two pairs of switches S12, S13, S14 and S15 are electrically connected to the at least two pairs of output terminals X1, X2, Y1 and Y2, respectively. The signal controller M2′ is configured to individually control output of the at least two alternating current signals from the at least two pairs of output terminals by individually controlling the at least two pairs of switches. FIG. 67 illustrates two pairs of switches S12, S13, S14 and S15. The pair of switches (S12, S13) is electrically connected to the pair of output terminals (X1, X2), and each of the switches is electrically connected to the corresponding output terminal, respectively. For example, switch S12 is electrically connected to output terminal X1 and switch S13 is electrically connected to output terminal X2. The pair of switches (S14, S15) and the pair of output terminals (Y1, Y2) are also electrically connected in a similar manner. Furthermore, the signal controller M2′ may control the output of X-direction alternating current signal and Y-direction alternating current signal from the pairs of output terminals (X1, X2) and (Y1, Y2) by individually controlling the pairs of switches (S12, S13) and (S14, S15). In various embodiments, the switches S11 to S15 may take any suitable form, such as electronic switches, mechanical switches, relays and the like.

In one example, when it is necessary to apply an X-direction electric field based on temperature information, the pair of switches (S12, S13) are closed. If it is not necessary to apply the X-direction electric field, the pair of switches (S12, S13) are opened, such that the pair of output terminals (X1, X2) cannot supply the X-direction alternating current signal for establishing the X-direction electric field. For the Y-direction electric field, it can also be controlled based on temperature information in a similar way. It should be known that, the control of the X-direction electric field will not interfere with the control of the Y-direction electric field, and vice versa.

To sum up, the electric field generators M and M′ may independently control the electric field applied to the corresponding body sites of the subject by individually controlling each pair of switches. For example, the electric field generators M and M′ may independently control the X-direction and Y-direction electric fields, which improves the utilization rate of electric fields and ensures the therapeutic effect.

In an exemplary embodiment, a signal controller, such as the signal controller M2 in FIG. 66 or the signal controller M2′ in FIG. 67, is configured to monitor the obtained temperature information according to each of the above-mentioned insulated electrodes 1 and 2. In response to the temperature information being greater than a temperature threshold, it controls to stop outputting the alternating current signal applied to that pair of insulated electrodes; and in response to the temperature information not being greater than the temperature threshold, it controls to output the alternating current signal applied to that pair of insulated electrodes among the at least two alternating current signals. In one example, the temperature threshold may be set as 41° C., which is the upper limit of the safe temperature of the human body surface. Therefore, when the temperature information monitored by an insulated electrode of a pair of insulated electrodes is greater than 41° C., the signal controller M2 may control to stop outputting the alternating current signal applied to the pair of insulated electrodes containing that insulated electrode. At the same time, when the temperature information monitored by a pair of insulated electrodes is not greater than 41° C., the signal controller M2 may control to continuously output the alternating current signal applied to that pair of insulated electrodes. The range of the temperature threshold is 37° C.-41° C.

In the present application, the actions of “controlling to stop outputting the alternating current signals” and “controlling to output the alternating current signals” may be realized by controlling the opening and closing of the corresponding switches S12, S13, S14 and S15, respectively. However, it will be understood that, these actions do not necessarily require explicit physical operations. For example, if a switch was originally closed to output an alternating current signal, it is not necessary to perform any explicit physical operation to control that switch to output an alternating current signal, but only to maintain that switch in a closed state, such as by maintaining the supply of a control signal for closing the switch.

To sum up, the electric field generators M and M′ of the present embodiment may independently control the output of alternating current signals based on the temperature information monitored by the insulated electrodes attached to the body surface of the subject through the signal controllers M2 and M2′, so as to ensure that the body temperature of the subject is within a safety threshold and avoid cryogenic burns.

FIG. 68 is a schematic block diagram of a tumor treating fields system 1000 according to an embodiment of the present application. Each of the insulated electrodes 1 and 2 may include a plurality of capacitively coupled electrodes. When the insulated electrodes 1 and 2 are placed on a subject's body, they may have good electrical contact with the body. Each of the insulated electrodes 1 and 2 has a temperature sensor array having a plurality of temperature sensors arranged on the insulated electrodes. The temperature sensor is configured to sense temperature signals of the adhesive of the insulated electrode attached to the corresponding body site to provide corresponding temperature information.

In one example embodiment, the tumor treating fields system 1000 further includes an adapter N. The adapter N is configured to convert the temperature signals from the temperature sensors of the insulated electrodes into temperature information, and transmit at least two alternating current signals to the corresponding at least two pairs of insulated electrodes. In one example, the temperature signals sensed by the temperature sensor arrays of at least two pairs of insulated electrodes are conducted into the adapter N for processing, so as to obtain the temperature information that can be used by the signal controller M2 of the electric field generator M. For example, the adapter N may process the voltage values sensed by the temperature sensors into corresponding temperature values for the signal controller M2 of the electric field generator M to make a further determination.

To sum up, the tumor treating fields system 1000 for applying an electric field to a subject may collect temperature signals and feed them back to the electric field generator M, and the electric field generator M controls the alternating electrical signals applied to the insulated electrodes based on the temperature information, thus ensuring the safety of the tumor treating fields system 1000 when applying alternating current signals. Because the electric field generator M of the present embodiment can independently control the electric fields in all directions, it also ensures that the tumor treating fields system 1000 can apply alternating current signals purposefully.

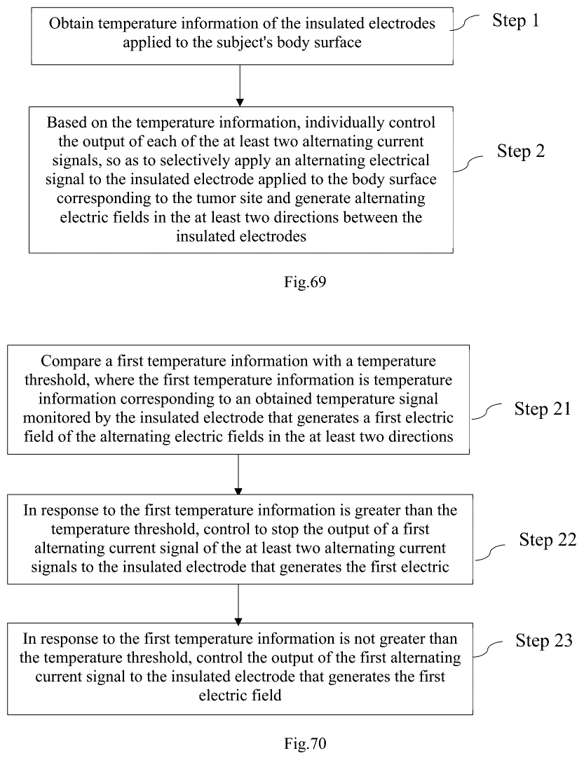

FIG. 69 is a schematic flow diagram of the electric field generator M of the tumor treating fields system 1000 applying an alternating electrical signal to the insulated electrode, including Step 10 and Step 20. The electric field generator is the electric field generator M shown in FIG. 66 or the electric field generator M′ shown in FIG. 67.

Step 10: Obtain temperature information of the insulated electrodes applied to the subject's body surface. Step 20: Based on the temperature information, individually control the output of each of the at least two alternating current signals, so as to selectively apply an alternating electrical signal to the insulated electrode attached to the body surface corresponding to the tumor site and generate an alternating electric field in the at least two directions between the insulated electrodes.

FIG. 70 is a flowchart of controlling the electric field generator M to apply alternating electrical signals to a pair of insulated electrodes in the step 20 shown in FIG. 69. In the step 20 shown in FIG. 69, individually controlling the output of each of the at least two alternating current signals, so as to selectively apply an alternating electrical signal to the insulated electrode attached to the body surface corresponding to the tumor site and generate alternating electric fields in the at least two directions between the insulated electrodes, includes the following steps.

Step 21: Compare first temperature information with a temperature threshold. The first temperature information is temperature information corresponding to an obtained temperature signal monitored by the insulated electrode that generate a first electric field of the alternating electric fields in the at least two directions.

Step 22: In response to the first temperature information being greater than the temperature threshold, control to stop the output of the first alternating current signal of the at least two alternating current signals to the insulated electrode that generates the first electric field.

Step 23: In response to the first temperature information being not greater than the temperature threshold, control to continuously output the first alternating current signal to the insulated electrode that generates the first electric field.

The range of the temperature threshold is 37° C.-41° C.

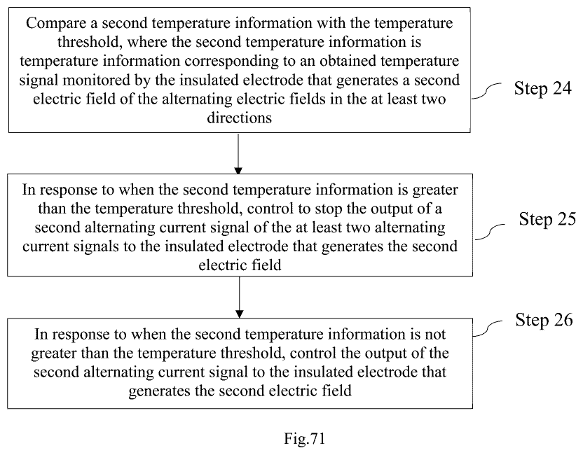

FIG. 71 is a further flowchart of controlling the electric field generator M to apply alternating current signals to the insulated electrodes in the step 20 shown in FIG. 69. The step 20 shown in FIG. 69 further includes the following steps.

Step 24: Compare a second temperature information with the temperature threshold. The second temperature information is temperature information corresponding to an obtained temperature signal monitored by the insulated electrode that generates a second electric field of the alternating electric fields in the at least two directions.

Step 25: In response to the second temperature information being greater than the temperature threshold, control to stop the output of the second alternating current signal of the at least two alternating current signals to the insulated electrode that generates the second electric field.

Step 26: In response to the second temperature information being not greater than the temperature threshold, control to continuously output the second alternating current signal to the insulated electrode that generates the second electric field.

The range of the temperature threshold is 37° C.-41° C.

In an exemplary embodiment, the electric field generator M continuously obtains the temperature information monitored and obtained by the insulated electrodes attached to the body surface of the tumor site, so as to control the output of the alternating current signals applied to the insulated electrode in real time.

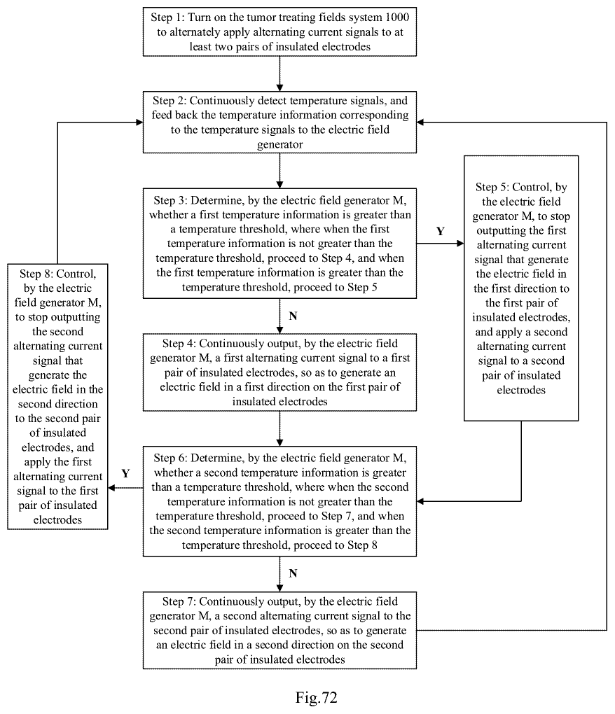

FIG. 72 is a flowchart of the operation of the tumor treating fields system 1000 of the present embodiment applying alternating electrical signals for tumor treatment. The method includes the following steps.

Step 1: Turn on the tumor treating fields system 1000 to alternately apply alternating current signals to at least two pairs of insulated electrodes.

Step 2: Continuously detect temperature signals, and feed back the temperature information corresponding to the temperature signals to the electric field generator.

Step 3: Determine, by the electric field generator M, whether a first temperature information is greater than a temperature threshold. When the first temperature information is not greater than the temperature threshold, it proceeds to Step 4. When the first temperature information is greater than the temperature threshold, it proceeds to Step 5.

Step 4: Continuously output, by the electric field generator M, a first alternating current signal to a first pair of insulated electrodes, so as to generate an electric field in a first direction on the first pair of insulated electrodes.

Step 5: Control, by the electric field generator M, to stop outputting the first alternating current signal that generates the electric field in the first direction to the first pair of insulated electrodes, and apply a second alternating current signal to a second pair of insulated electrodes.

Step 6: Determine, by the electric field generator M, whether a second temperature information is greater than a temperature threshold. When the second temperature information is not greater than the temperature threshold, it proceeds to Step 7. When the second temperature information is greater than the temperature threshold, it proceeds to Step 8.

Step 7: Continuously output, by the electric field generator M, the second alternating current signal to the second pair of insulated electrodes, so as to generate an electric field in a second direction on the second pair of insulated electrodes.

Step 8: Control, by the electric field generator M, to stop outputting the second alternating current signal that generates the electric field in the second direction to the second pair of insulated electrodes, and apply the first alternating current signal to the first pair of insulated electrodes.

The alternating current signal alternately applied in Step 1 includes: the first alternating current signal and the second alternating current signal. The first alternating current signal and the second alternating current signal are both sine wave signals, and have the same frequency and the same peak value of the AC voltage amplitude.

The temperature signal in Step 2 is the temperature signal of the adhesive monitored and obtained by the temperature sensors of the insulated electrodes when applying alternating current signals. The first temperature information in Step 3 is obtained by the electric field generator M or the adapter processing the feedbacked temperature signals of the first pair of insulated electrodes. The second temperature information in Step 6 is obtained by the electric field generator M or the adapter processing the feedbacked temperature signals of the second pair of insulated electrodes. The range of the temperature threshold in Step 3 and Step 6 is 37° C.-41° C. The electric field in the first direction in Step 4 is perpendicular to the electric field in the second direction in Step 7.

During the process of the tumor treating fields system 1000 applying alternating electrical signals through insulated electrodes, when any detected temperature information exceeds the temperature threshold, the electric field generator M will turn off the alternating electrical signal applied to the pair of insulated electrodes until the temperature information of that pair of insulated electrodes returns to normal. However, turning off the output of the alternating current signal applied on one pair of insulated electrodes does not affect the output of the alternating electrical signal applied on the other pair of insulated electrodes. That is, when the temperature information on one pair of insulated electrodes exceeds the threshold, the alternating current signal generated by the treating fields device is switched to the other pair of insulated electrodes, which can ensure the continuous application of alternating electrical signals to the tumor site and ensure the therapeutic effect.

The present embodiment further provides a computer-readable storage medium having instructions stored thereon. When the instructions are executed by the signal controller M2 of the electric field generator M as mentioned above, the electric field generator M is caused to perform the above-mentioned method.

The present embodiment further provides a computer program product comprising instructions. When the instructions are executed by the signal controller M2 of the electric field generator M as mentioned above, the electric field generator is caused to perform the above-mentioned method.

Referring to FIG. 1 and FIG. 68, a plurality of temperature sensor arrays are configured on a plurality of insulated electrodes 1 and 2 and configured to sense temperature signals of adhesives of insulated electrodes that are adhered to the corresponding body sites to provide corresponding temperature information. In some exemplary embodiments, each temperature sensor array of the plurality of temperature sensor arrays (not labeled) includes a plurality of thermistors. Each thermistor may sense the temperature of the adhesive adhered to the corresponding body site and generate a corresponding analog voltage value. When the insulated electrodes 1 and 2 are adhered to a subject's body, they may be stuck to the predetermined position on the surface of the skin through their backing and the adhesive (such as the conductive gel).

The insulated electrodes 1 and 2 further include a first cable H1. The first cable H1 is configured to provide an alternating electric field signal path (not labeled) and a plurality of temperature signal paths (not labeled). In one example, the first cable H1 can be a cable of copper with ten cores, in which eight cores represent eight temperature signal paths for transmitting temperature information generated by eight temperature sensors; one core represents a first electric field 3 signal path for transmitting the alternating electric field signal generated by the electric field generator M; and one core is used for grounding.

The adapter Nis configured to: transmit the plurality of alternating electric field signals generated by the electric field generator M to the plurality of insulated electrodes 1 and 2 through an alternating electrical signal path (not labeled), so as to apply alternating electric fields to corresponding body sites; and receive corresponding temperature signals transmitted in parallel through the plurality of temperature signal paths (not labeled), and transmit the plurality of temperature values corresponding to the corresponding temperature signals to the electric field generator M, so that the electric field generator M may control the plurality of alternating electric field signals based on the plurality of temperature values. In one example, the adapter N may be located between the electric field generator M and each of the insulated electrode 1 and 2. The adapter N may be electrically connected to the electric field generator M and transmit temperature values to the electric field generator M. The adapter N may also be electrically connected with each of the insulated electrodes 1 and 2 and transmit the plurality of alternating electric field signals generated by the electric field generator M to the corresponding insulated electrodes 1 and 2.

To sum up, the temperature sensors are connected with the adapter N in parallel through the first cable H1. Therefore, the temperature signals generated by the temperature sensors may be input in parallel to the adapter N. Compared with the scheme of serial transmission of temperature signals, the transmission speed is improved, so as to facilitate to monitor the temperature of the applied surface of each of the insulated electrode 1 and 2 in real-time.

In some exemplary embodiments, the tumor treating fields system 1000 further includes a second cable H2. The second cable H2 is configured to transmit a plurality of alternating electric field signals from the electric field generator M to the adapter N. In one example, the second cable H2 may be a cable of copper with eight cores and the cable of copper is wrapped with a shielding layer. Among them, four cores are AC lines X1, X2, Y1, Y2 that transmit alternating electrical signals to four insulated electrodes 1 and 2; one core is a serial data transmission line TX; one core is a serial data reception line RX; one core is a VCC line that provides direct current to the adapter N; and one core is a grounding signal line GND. The serial data transmission line TX is used to transmit the temperature signals obtained by the temperature sensors of the corresponding insulated electrodes 1 and 2 to the electric field generator M, while the serial data reception line RX is used to transmit the control signals of the electric field generator M to the corresponding modules. As a result, a plurality of alternating electric field signals generated by the electric field generator M may be transmitted to four cores of the first cables H1 through the four cores of the second cable H2, and applied to the four insulated electrodes 1 and 2.

The insulated electrodes 1 and 2 further include a first connector J1. The first connector J1 mechanically and electrically connects the first cable H1 to the adapter N. In one example, the first connector J1 may include a press-type spring connector to facilitate quick replacement between the adapter N and the insulated electrodes 1 and 2.

In some exemplary embodiments, the tumor treating fields system 1000 further includes a second connector J2. The second connector J2 is configured to mechanically and electrically connect the second cable H2 to the electric field generator M. In one example, the second connector J2 may include a press-type spring connector.

To sum up, the tumor treating fields system 1000 including the first connector J1, the second connector J2, and the second cable H2 may facilitate the attachment of the insulated electrodes 1 and 2 to a subject's skin by the subject or a nursing staff, avoiding obstruction due to the presence of the cable.

In some exemplary embodiments, the temperature signal generated by the temperature sensor is an analog signal, and thus it is necessary to perform analog-to-digital conversion on the temperature signal for subsequent computations.

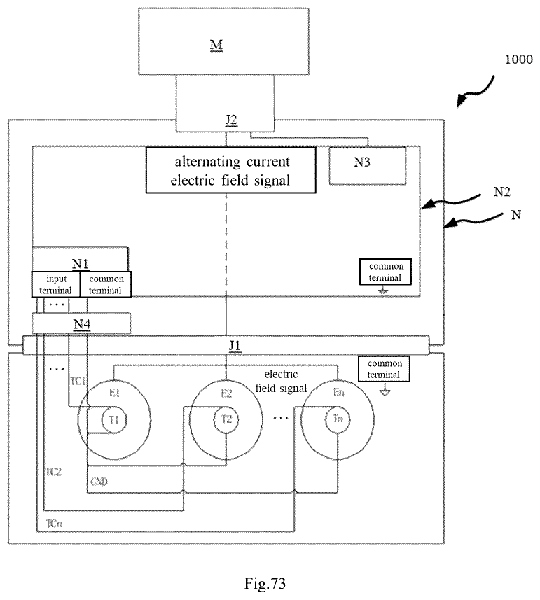

FIG. 73 is another schematic block diagram of the electric field generator M of the tumor treating fields system 1000. It will be understood that, for the sake of clarity in the diagram, FIG. 73 only illustrates one insulated electrode and one temperature sensor array (T1, T2 . . . Tn) on the insulated electrode, but the present disclosure is not limited to this.

As shown in FIG. 73, the adapter N includes: an analog-to-digital converter N1 and a signal processor N2. The analog-to-digital converter N1 is configured to convert the corresponding temperature signals into digital signals, and the signal processor N2 is configured to calculate and store a plurality of temperature values based on the digital signals. In one example, the analog-to-digital converter N1 may choose an analog-to-digital conversion integrated circuit with a communication protocol (such as SPI, 12C, etc.) to form digital signals from the temperature signals collected digitally, and provide the digital signals to the signal processor N2 for processing. In one example, the signal processor N2 is electrically connected with the analog-to-digital converter N1 and receives the digital signals. In one example, the signal processor N2 may choose an integrated circuit with data computation and storage capability (such as microcontrollers, FPGAs, etc.) to calculate the plurality of temperature values based on the digital signals. In some other examples, the signal processor N2 may also use processors with built-in analog-to-digital converters and serial communication protocols (such as the STM32F103 series MCU) to simplify the circuit structure.

In some exemplary embodiments, the adapter N further includes a serial communication circuit N3. The serial communication circuit N3 is configured to serially transmit the plurality of temperature values to the electric field generator M. In one example, the serial communication circuit N3 may choose an integrated circuit with a serial communication protocol (such as RS232, RS485, etc.) for transmitting the plurality of temperature values.

In some exemplary embodiments, each temperature sensor array of the plurality of temperature sensor arrays includes a plurality of thermistors T1, T2 . . . Tn. By using the thermistor, it may determine the temperature of the corresponding insulated electrode by measuring the voltage value of the thermistor, and the thermistor may be flexibly configured to contact the human body due to its small size.

In some exemplary embodiments, each temperature sensor array may directly transmit the voltage signal of the thermistor in parallel to the analog-to-digital converter N1. For example, in an array consisting of 8 thermistors, the positive-end copper wires (TC1, TC2 . . . TCn) of each thermistor is connected in parallel to the analog-to-digital converter N1. In some exemplary embodiments, the positive-end copper wires (TC1, TC2 . . . TCn) of each thermistor may be connected to the analog-to-digital converter N1 in parallel through the first connector J1.

In some exemplary embodiments, the adapter N further includes a buffer N4. The buffer N4 includes: a plurality of input terminals, electrically connected to corresponding thermistors of the plurality of thermistors; and a plurality of output terminals, electrically connected to corresponding input terminals of the analog-to-digital converter N1. In some exemplary embodiments, the positive-end copper wires (TC1, TC2 . . . TCn) of each thermistor may be connected to the plurality of input terminals of the buffer N4 in parallel through the first connector J1, and common terminals of the thermistor may be cascaded together and then connected to the adapter N.

In some exemplary embodiments, the buffer N4 may be composed of an operational amplifier circuit for isolating the preceding signal to protect the analog-to-digital converter N1 at its back end. In one example, the buffer N4 may use a voltage follower circuit.

In some exemplary embodiments, each insulated electrode may include a plurality of dielectric elements E1, E2 . . . En. In one example, the alternating current electric field signal may be applied to the dielectric elements E1, E2 . . . En through the adapter N, the first connector J1, and the first cable H1 (not shown in FIG. 73).

To sum up, each thermistor T1, T2 . . . Tn in the temperature sensor array is electrically connected to the analog-to-digital converter N1 in parallel. They may also be connected to the analog-to-digital converter N1 through the first connector J1 and the buffer N4. For example, if four groups of temperature sensor arrays are used and each group contains eight thermistors, then 32 thermistors will be sent to the analog-to-digital converter N1 in parallel through the buffer N4, improving the transmission rate.

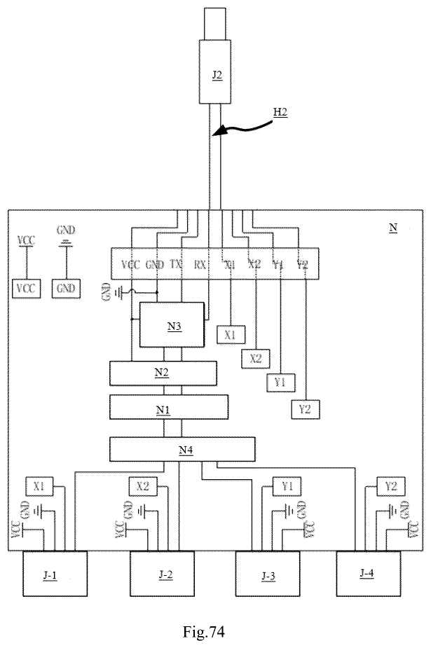

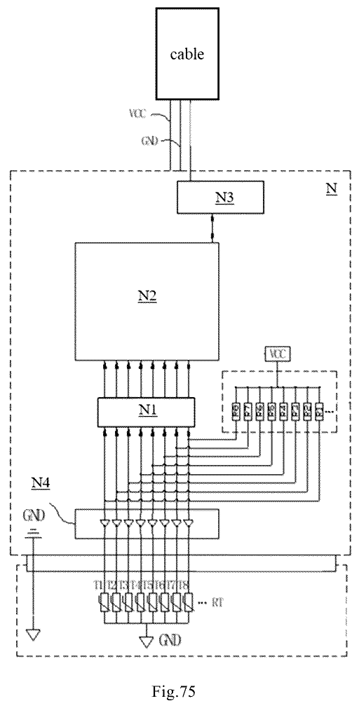

FIG. 74 is a schematic block diagram of the internal structure of the adapter N shown in FIG. 73. As shown in FIG. 74, in one example, the second cable H2 is an eight-core copper wire and is connected to the electric field generator M through the second connector J2. Herein, the eight-core copper wire corresponds to 8 signals, namely: power VCC, grounding GND, serial data transmitting end TX, serial data receiving end RX, alternating current signal X1, alternating current signal X2, alternating current signal Y1, and alternating current signal Y2. The power VCCs shown in FIG. 74 are all connected, and the groundings GND are also connected. The alternating current signals X1, X2, Y1, and Y2 are connected with corresponding dotted terminals. The power VCC, ground GND, and the alternating current signals in the adapter N are all transmitted to the plurality of insulated electrodes through the first cable H1 respectively connected to the first connector ports J-1, J-2, J-3, and J-4.

In the example of FIG. 74, as previously described, the adapter N includes: an analog-to-digital converter N1, a signal processor N2, a serial communication circuit N3, and a buffer N4. In this example, the analog-to-digital converter N1 and the serial communication circuit N3 are shown to be separated from the signal processor N2. However, as afore-mentioned, in some embodiments, the signal processor N2 may have the analog-to-digital converter N1 and the serial communication circuit N3 built-in to simplify the circuit structure. The temperature signals from the plurality of temperature sensor arrays are transmitted in reverse to the buffer N4 through ports J-1, J-2, J-3, and J-4 of the first connector J1 connected to the respective corresponding first cable H1. The temperature signals are then transmitted to the analog-to-digital converter N1 to convert into digital signals, and are then transmitted to the signal processor N2 for calculating the temperature values in the signal processor N2. The temperature values are then transmitted to the serial communication circuit N3 (such as RS232) by the signal processor N2. Finally, the serial communication circuit N3 sends the temperature value data to the electric field generator M in series.

FIG. 75 is a schematic circuit diagram of the adapter N shown in FIG. 73. The adapter N includes an analog-to-digital converter N1, a signal processor N2, and a serial communication circuit N3. In this example, the analog-to-digital converter N1 and the serial communication circuit N3 are shown to be separated from the signal processor N2. However, as afore-mentioned, in some embodiments, the signal processor N2 may have the analog-to-digital converter N1 and the serial communication circuit N3 built-in to simplify the circuit structure.

As shown in FIG. 75, each temperature sensor array of a plurality of temperature sensor arrays includes a plurality of thermistors T1-T8. The positive ends of the thermistor T1-T8 are electrically connected in parallel to input ports of the analog-to-digital converter N1. In some exemplary embodiments, the adapter N further includes a voltage regulator VCC and a plurality of precision resistors R1-R8. The plurality of precision resistors R1-R8 are electrically connected between the voltage regulator VCC and the corresponding thermistor of the plurality of thermistors T1-T8. For example, the precision resistor R1 is connected between the voltage regulator VCC and the thermistor T1. In some exemplary embodiments, the adapter N further includes a buffer N4. In one example, a plurality of input terminals of the buffer N4 are electrically connected to the plurality of thermistors T1-T8 respectively, and a plurality of output terminals of the buffer N4 are further electrically connected to the corresponding precision resistor of the plurality of precision resistors R1-R8.

Since a change in temperature may synchronously cause a change in the resistance value of the thermistor, by connecting the precision resistor R_{S} and the voltage regulator VCC, the thermistor and precision resistor are equivalent to two resistors connected in series for voltage division. The relationship between the resistance value RT of the thermistor and the voltage V_{RT} satisfies:

$${V}_{R T} = V C C \times ( {R}_{T} / ( {R}_{T} + {R}_{S} ) )$$

Wherein, V_{RT} is the resistance value of the thermistor at temperature T(K), and R_{S} is the resistance value of the precision resistor connected with the thermistor.

It can be seen that, when the resistance value R_{T} of the thermistor decreases due to temperature changes, the collected voltage V_{RT} also decreases. Since the voltage V_{RT} is an analog quantity, it is converted into a digital signal through an analog-to-digital converter N1. The signal processor N2 calculates the current temperature value based on the digital signal, where the relationship between the resistance value R_{T} of the thermistor and the voltage V_{RT} satisfies:

$${R}_{T} = {R}_{N} \times {e}^{B ( \frac{1}{T} - \frac{1}{{T}_{N}} )}$$

Wherein, R_{N} is the resistance value of the thermistor at the rated temperature T_{N}(K); T is the target temperature (K) in Kelvin, B is the thermal coefficient of the thermistor; and e is a constant (2.71828). For example, when a 3.3V power supply (VCC) and a thermistor with B=3380 is used, R_{N} is 10K at 25° C. When the collected voltage V_{RT} is 1.5022V, the obtained R_{T} is about 8355.88 ohms. Meanwhile, the target T is calculated to be 29.8° C. In one example, the analog-to-digital converter N1 uses a 12-bit analog-to-digital conversion chip. Under a 3.3V power supply voltage, the minimum measured voltage is about 0.8056 mV, and the corresponding minimum temperature resolution is about 0.03° C. The accuracy of the measured temperature values is high. In addition, four groups of 32-channel thermistors T1, T2, etc. transmit voltage signals to the analog-to-digital converter N1 in parallel, and then the voltage signals are processed by a signal processor N2 and transmitted through a serial communication circuit N3, improving the transmission rate.

Although the present disclosure has been illustrated and described in detail in the drawings and the foregoing description, such illustration and description should be regarded as illustrative and schematic, and not restrictive. The present disclosure is not limited to the disclosed embodiments. By studying the drawings, the disclosure and the appended claims, those skilled in the art should understand and realize variations to the disclosed embodiments when practicing the claimed subject matter. In the claims, the term “comprising”/“comprises” does not exclude other elements or steps not listed; the indefinite article “a” or “an” does not exclude a plurality, and the term “a plurality of” refers to two or more. The mere fact that certain measures are recited in mutually different dependent claims does not indicate that a combination of these measures cannot be used to advantage.

1. A tumor treating fields system including an electric field generator and at least two pairs of insulated electrodes, wherein the electric field generator comprises:
an alternating current signal generator, configured to generate at least two alternating current signals, wherein the at least two alternating current signals are output to the at least two pairs of insulated electrodes, so as to generate alternating electric fields in at least two directions between the at least two pairs of insulated electrodes; and
a signal controller, configured to obtain temperature information of the insulated electrodes, and individually control output of each of the at least two alternating current signals based on the temperature information, so as to selectively apply to corresponding insulated electrodes an alternating current signal that generates a corresponding alternating electric field of the alternating electric fields in the at least two directions.

2. The tumor treating fields system according to claim 1, wherein the alternating current signal generator comprises:
a direct current signal source, configured to generate a direct current signal; and
a signal converter, configured to convert the direct current signal into the at least two alternating current signals.

3. The tumor treating fields system according to claim 2, wherein the alternating current signal generator further comprises:
a direct current signal switch, electrically connected between the direct current signal source and the signal converter,
wherein the signal controller is configured to control a supply of the direct current signal from the direct current signal source to the signal converter by controlling the direct current signal switch.

4. The tumor treating fields system according to claim 1, further comprising:
at least two pairs of output terminals, wherein each pair of output terminals is configured for supplying corresponding alternating current signal of the at least two alternating current signals from the alternating current signal generator.

5. The tumor treating fields system according to claim 4, further comprising:
at least two pairs of switches, electrically connected to the at least two pairs of output terminals respectively,
wherein the signal controller is configured to individually control output of the at least two alternating current signals from the at least two pairs of output terminals by individually controlling the at least two pairs of switches.

6. The tumor treating fields system according to claim 1, wherein the signal controller is configured to:
monitor the obtained temperature information according to each of the insulated electrodes;
in response to the temperature information being greater than a temperature threshold, control to stop outputting the alternating current signal applied to the corresponding insulated electrode among the at least two alternating current signals; and
in response to the temperature information not being greater than the temperature threshold, control to output the alternating current signal applied to the corresponding insulated electrode among the at least two alternating current signals.

7. The tumor treating fields system according to claim 6, wherein a range of the temperature threshold is 37° C.-41° C.

8. The tumor treating fields system according to claim 1, further comprising:
at least two pairs of temperature sensor arrays, configured to sense temperature signals of the insulated electrodes, so as to provide corresponding temperature information.

9. The tumor treating fields system according to claim 8, further comprising:
an adapter, configured to convert the temperature signals into the temperature information, and transmit the at least two alternating current signals to the at least two pairs of the insulated electrodes.

10. A method of a tumor treating fields system applying alternating electrical signals, wherein the tumor treating fields system is the tumor treating fields system according to claim 1, and the method comprises the following steps:
step 10: obtaining temperature information of the insulated electrodes; and
step 20: based on the temperature information, individually controlling output of each of the at least two alternating current signals, so as to selectively apply to an insulated electrode an alternating current signal corresponding to an alternating electric field of the alternating electric fields in the at least two directions.

11. The method according to claim 10, wherein the step 20 comprises the following steps:
step 21: comparing a first temperature information with a temperature threshold, wherein the first temperature information is temperature information corresponding to an obtained temperature signal monitored by the insulated electrode that generates a first electric field of the alternating electric fields in the at least two directions;
step 22: in response to the first temperature information being greater than the temperature threshold, controlling to stop output of a first alternating current signal of the at least two alternating current signals to the insulated electrode that generates the first electric field; and
step 23: in response to the first temperature information not being greater than the temperature threshold, controlling to continuously output the first alternating current signal to the insulated electrode that generates the first electric field.

12. The method according to claim 11, wherein a range of the temperature threshold is 37° C.-41° C.

13. The method of claim 10, wherein the step 20 further comprises the following steps:
step 24: comparing a second temperature information with the temperature threshold, wherein the second temperature information is temperature information corresponding to an obtained temperature signal monitored by the insulated electrode that generates a second electric field of the alternating electric fields in the at least two directions;
step 25: in response to the second temperature information being greater than the temperature threshold, controlling to stop output of a second alternating current signal of the at least two alternating current signals to the insulated electrode that generates the second electric field; and
step 26: in response to the second temperature information not being greater than the temperature threshold, controlling to continuously output the second alternating current signal to the insulated electrode that generates the second electric field.

14. The method according to claim 13, wherein a range of the temperature threshold is 37° C.-41° C.

15. A computer-readable storage medium having instructions stored thereon, the instructions, when being executed by the signal controller of the electric field generator of the tumor treating fields system according to claim 1, causing the electric field generator a method of a tumor treating fields system applying alternating electrical signals, the method comprises the following steps:
step 10: obtaining temperature information of the insulated electrodes; and step 20: based on the temperature information, individually controlling output of each of the at least two alternating current signals, so as to selectively apply to an insulated electrode an alternating current signal corresponding to an alternating electric field of the alternating electric fields in the at least two directions.

16. A computer program product comprising instructions, the instructions, when being executed by the signal controller of the electric field generator of the tumor treating fields system according to claim 1, causing the electric field generator to perform a method of a tumor treating fields system applying alternating electrical signals, the method comprises the following steps:
step 10: obtaining temperature information of the insulated electrodes; and
step 20: based on the temperature information, individually controlling output of each of the at least two alternating current signals, so as to selectively apply to an insulated electrode an alternating current signal corresponding to an alternating electric field of the alternating electric fields in the at least two directions.
